(19) United States
(12) Patent Application Publication
(10) Pub. No.: US 2025/0145561 A1
(43) Pub. Date: May 8, 2025

(54) 4-SUBSTITUTED-PHENYL ACETAMIDES AND ARYLUREAS AS AGONISTS FOR THE ORPHAN RECEPTOR GPR88

(71) Applicant: RESEARCH TRIANGLE INSTITUTE, Research Triangle Park, NC (US)

(72) Inventors: Chunyang JIN, Apex, NC (US); Md Toufiqur RAHMAN, Cary, NC (US); Dongliang GUAN, Durham, NC (US); Chaminda Lakmal HETTI HANDI, Durham, NC (US); Ann Marie DECKER, Durham, NC (US)

(73) Assignee: RESEARCH TRIANGLE INSTITUTE, Research Triangle Park, NC (US)

(21) Appl. No.: 18/832,872

(22) PCT Filed: Jan. 23, 2023
(86) PCT No.: PCT/US2023/011330

§ 371 Date: Jul. 24, 2024

Related U.S. Application Data
(60) Provisional application No. 63/302,835, filed on Jan. 25, 2022.
(60) Provisional application No. 63/419,470, filed on Oct. 26, 2022.

(51) Int. Cl. C07C 235/34; A61K 31/165; A61K 31/167; A61K 31/17; A61K 31/216; A61K 31/341; A61K 31/381; A61K 31/40; A61K 31/4192; A61K 31/4196; A61K 31/4402; C07C 233/11; C07C 235/36; C07C 235/38; C07C 235/78; C07C 275/40; C07D 207/09; C07D 213/40; C07D 249/04; C07D 249/08; C07D 307/52; C07D 333/20

(52) U.S. Cl. CPC C07C 235/34; A61K 31/165; A61K 31/167; A61K 31/17; A61K 31/216; A61K 31/341; A61K 31/381; A61K 31/40; A61K 31/4192; A61K 31/4196; A61K 31/4402; C07C 233/11; C07C 235/36; C07C 235/38; C07C 235/78; C07C 275/40; C07D 207/09; C07D 213/40; C07D 249/04; C07D 249/08; C07D 307/52; C07D 333/20; C07C 2601/02; C07C 2601/04

(57) ABSTRACT

The present disclosure provides novel 4-substituted-phenyl acetamide and arylurea derivatives that act as agonists against GPR88. The compounds of the present disclosure are believed to be useful for the treatment of diseases and conditions caused by physiological processes implicating the orphan receptor GPR88, including diverse brain and behavioral functions such as cognition, mood, movement control, and reward-based learning. GPR88 is emerging as a novel drug target for central nervous system disorders including schizophrenia, Parkinson's disease, anxiety, and addiction. One particular indication for which GPR88 agonists hold promise is alcohol use disorder (AUD).

### CROSS-REFERENCE TO RELATED APPLICATIONS

This application claims priority to U.S. Provisional Patent Application Ser. No. 63/302,835 filed on Jan. 25, 2022, and U.S. Provisional Patent Application Ser. No. 63/419,470 filed on Oct. 26, 2022, both of which are hereby incorporated by reference in their entirety.

### FEDERALLY SPONSORED RESEARCH

This disclosure was made with government support under R01AA026820 awarded by NIH. The government has certain rights in the disclosure.

### FIELD OF THE INVENTION

The a present disclosure provides novel 4-substituted-phenyl acetamide derivatives that act as agonists against GPR88. The compounds of the present disclosure are believed to be useful for the treatment of diseases and conditions caused by physiological processes implicating the orphan receptor GPR88, including diverse brain and behavioral functions such as cognition, mood, movement control, and reward-based learning. GPR88 is emerging as a novel drug target for central nervous system disorders including schizophrenia, Parkinson's disease, anxiety, addiction, and attention-deficit/hyperactivity disorder (ADD/ADHD). One particular indication for which GPR88 agonists hold promise is alcohol use disorder (AUD).

### BACKGROUND OF THE DISCLOSURE

Alcoholism is a heterogeneous, chronic relapsing disorder, and exacts great emotional, social, and economic costs. See, e.g., Rehm et al., Global burden of disease and injury and economic cost attributable to alcohol use and alcohol-use disorders. Lancet2009, 373, 2223-2233, and Thavorncharoensap et al., The economic impact of alcohol consumption: a systematic review. Substance Abuse Treatment, Prevention, and Policy 2009, 4, 20, each of which is incorporated herein by reference with regard to such background teaching. In the United States, alcohol is the third leading preventable cause of death, and the cost of alcoholism reached $249 billion in 2010 (CDC). At the neurobiological level, alcohol activates multiple, primary and secondary molecular targets in the brain and triggers broad alterations in gene expression and synaptic plasticity. See, e.g., Costardi et al., A review on alcohol: from the central action mechanism to chemical dependency. Revista da Associacao Medica Brasileira 2015, 61, 381-387, which is incorporated herein by reference with regard to such background teaching. Primarily, alcohol interacts with GABA_{A} and NMDA ion channel receptors, mediating gamma-aminobutyric acid (GABA) and glutamate neurotransmissions. Alcohol enhances the GABA action and antagonizes glutamate action, therefore acting as a central nervous system (CNS) depressant. In addition, both dopamine and opioid peptide systems in the basal forebrain have been implicated in alcohol reward. Accordingly, the competitive opioid antagonist naltrexone and the functional glutamate antagonist acamprosate provide proof of concept for pharmacotherapy of alcohol addiction and dependence. See, e.g., Bouza et al., Efficacy and safety of naltrexone and acamprosate in the treatment of alcohol dependence: a systematic review. Addiction 2004, 99, 811-828, which is incorporated herein by reference with regard to such background teaching. Both drugs, however, face significant challenges including lack of adherence, low efficacy, and serious side effects. In addition, alcohol-dependent individuals represent a heterogeneous group, and it is unlikely that a single pharmacological treatment will be effective for all alcoholic patients. Hence, novel therapeutic strategies are needed for successful treatment of alcoholism.

GPR88 is an orphan G protein-coupled receptor (GPCR) coupled to Gα_{i/o} proteins and has robust expression in the striatal medium spiny neurons (MSNs) throughout the dorsal and ventral areas. See, e.g., Mizushima et al, A novel G-protein-coupled receptor gene expressed in striatum. Genomics 2000, 69, 314-321, Van Waes et al., GPR88—a putative signaling molecule predominantly expressed in the striatum: Cellular localization and developmental regulation. Basal Ganglia 2011, 1, 83-89, and Massart et al., Striatal GPR88 expression is confined to the whole projection neuron population and is regulated by dopaminergic and glutamatergic afferents. European Journal of Neuroscience 2009, 30, 397-414, each of which is incorporated herein by reference with regard to such background teaching. This distribution is consistent with transcriptional profiling studies linking GPR88 with a variety of striatal disorders, including Parkinson's disease, schizophrenia, and drug addiction. See, e.g., Ye et al., Orphan receptor GPR88 as an emerging neurotherapeutic target. ACS Chemical Neuroscience 2019, 10, 190-200, which is incorporated herein by reference with regard to such background teaching. More direct evidence of GPR88 functions is obtained from GPR88 knockout (KO) studies, suggesting that GPR88 plays an important role in regulating striatal functions involved in the reward system. See, e.g., Logue et al., The orphan GPCR, GPR88, modulates function of the striatal dopamine system: a possible therapeutic target for psychiatric disorders?Molecular and Cellular Neuroscience 2009, 42, 438-447, and Quintana et al., Lack of GPR88 enhances medium spiny neuron activity and alters motor- and cue-dependent behaviors. Nature Neuroscience 2012, 15, 1547-1555, each of which is incorporated herein by reference with regard to such background teaching. Of direct relevance to alcohol addiction, GPR88 KO mice displayed enhanced voluntary alcohol drinking in both moderate and excessive drinking paradigms compared with wild-type mice. See, e.g., Ben Hamida, et al., Increased alcohol seeking in mice lacking Gpr88 involves dysfunctional mesocorticolimbic networks. Biological Psychiatry 2018, 84, 202-212, which is incorporated herein by reference with regard to such background teaching. Importantly, no alterations in water intake, palatability and alcohol metabolism were observed in these mice. Moreover, mice lacking the GPR88 gene showed enhanced motivation for alcohol-taking and -seeking behaviors using operant self-administration. In agreement with the results from the KO studies, the inventors recently developed a potent, highly selective, and brain-penetrant GPR88 agonist, referred to as RTI-13951-33, which significantly reduced alcohol self-administration and alcohol intake in a dose-dependent manner without effects on locomotor activity and sucrose self-administration in rats when administered intraperitoneally. See, e.g., Jin et al., Discovery of a potent, selective, and brain-penetrant small molecule that activates the orphan receptor GPR88 and reduces alcohol intake. Journal of Medicinal Chemistry 2018, 61, 6748-6758, which is incorporated herein by reference with regard to such background teaching. Given the lack of modulatory effect on sucrose self-administration at doses that reduced alcohol self-administration, this suggests that the GPR88 agonist had a selective effect for alcohol reinforcement. Taken together, both genetic modification and pharmacological studies support the use of GPR88 agonists as a novel therapeutic tool to treat alcohol addiction and related disorders.

In order to understand the pharmacology of the GPR88 receptor for therapeutic application, development of structurally different chemotypes showing potent and selective GPR88 agonism is of great importance. In this regard, a compound known as 2-AMPP [(2S)—N-((1 R)-2-amino-1-(4-(2-methylpentyloxy)-phenyl)ethyl)-2-phenylpropanamide (1)] and its hydroxyl analogue 2 advanced knowledge in the relevant pharmacophore:

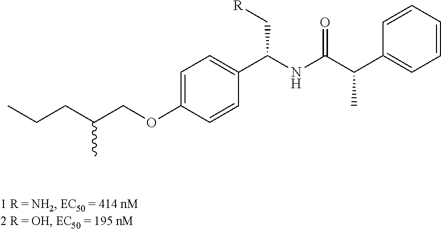

Reference is made to WO 2011/044195A1, WO 2011/044225A1, US 2011/0251196A1, US 2011/0245264 A1, and Dzierba et al., Design, synthesis, and evaluation of phenylglycinols and phenyl amines as agonists of GPR88. Bioorganic & Medicinal Chemistry Letters 2015, 25, 1448-1452, each of which is incorporated by reference with regard to such teaching. In a Lance time-resolved fluorescence resonance energy transfer (TR-FRET) cAMP assay with stable CHO-GPR88 cells, compounds 1 and 2 had moderate agonist activity at GPR88 with EC₅₀ values of 414 nM and 195 nM, respectively. See, e.g., Jin et al., Design, synthesis, and pharmacological evaluation of 4-hydroxyphenylglycine and 4-hydroxyphenylglycinol derivatives as GPR88 agonists. Bioorganic & Medicinal Chemistry 2017, 25, 805-812, which is incorporated by reference herein with regard to such teaching. These EC₅₀ values are slightly less potent than the previously reported EC₅₀ values, supra, which is possibly due to the relative sensitivity of the two different assay systems.

The inventors explored the structure-activity relationship and discovered a series of novel reversed amides and urea analogs of the 2-AMPP scaffold, which exhibited good to moderate agonist activity at GPR88. See, e.g., Jin et al., supra, Rahman, et al., Design, synthesis, and structure-activity relationship studies of (4-alkoxyphenyl)glycinamides and bioisosteric 1,3,4-oxadiazoles as GPR88 agonists. Journal of Medicinal Chemistry 2020, 63, 14989-15012, and Rahman, et al., Evaluation of amide bioisosteres leading to 1,2,3-triazole containing compounds as GPR88 agonists: Design, synthesis, and structure-activity relationship studies. Journal of Medicinal Chemistry 2021, 64, 12397-12413, each of which is incorporated by reference with regard to such teaching.

The present disclosure provides novel 4-substituted-phenyl acetamide and arylurea derivatives that act as agonists against GPR88, the compounds having structural differences from known compounds that offer unexpected benefits, including potency for the GPR88 target.

### BRIEF SUMMARY OF THE DISCLOSURE

The compounds of the present disclosure offer potent agonism to the orphan receptor GPR88. As such, the compounds of the present disclosure provide therapeutic potential for the treatment of striatal associated disorders such as Parkinson's disease, schizophrenia, and alcohol addiction. All compounds were evaluated for the GPR88 agonist activity in the inventors' previously established in vitro GPR88 Lance TR-FRET cAMP assay. See, supra, Jin et al., 2018, Rahman et al., 2020, and Rahman et al., 2021. As will be described, the TR-FRET signal was converted to fmol cAMP by interpolating from the standard cAMP curve. Fmol cAMP was plotted against the log of compound concentration and data were fit to a three-parameter logistic curve to generate maximum response (Eₘₐₓ) and EC₅₀ values.

One embodiment of the present disclosure includes a compound of Formula (I) or a pharmaceutically acceptable salt thereof:

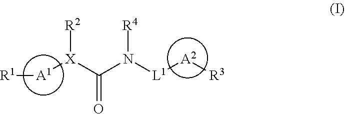

wherein
R¹ is selected from the group consisting of hydrogen, C₁₋₁₀ alkyl, C₂₋₁₀ alkenyl, C₂₋₁₀ alkynyl, C₁₋₁₀ alkoxy, C₁₋₁₀ haloalkyl, NH₂, NHC₁₋₁₀ alkyl, N(C₁₋₁₀ alkyl)₂, S(O)C₁₋₁₀ alkyl, S(O)₂C₁₋₁₀ alkyl, NHSO₂C₁₋₁₀ alkyl, C₃₋₆ cycloalkyl, 5-7 membered heterocycle, C₆₋₁₀ aryl, and C₅₋₁₀ heteroaryl;
A¹ is
   a) a 6-membered aromatic ring which may contain one N heteroatom; or
   b) a 10-membered fused aromatic ring system which may contain one or two N heteroatoms;
X is CH or N;
R² is (CH₂)ₙ₂R²⁰;
n2 is 0, 1, 2, or 3;
R²⁰ is selected from the group consisting of hydrogen, C₁₋₆ alkyl, C₂₋₆ alkenyl, C₂₋₆ alkynyl, OH, C₁₋₆ alkoxy, halogen, C₁₋₆ haloalkyl, NO₂, CN, NH₂, NHC₁₋₆ alkyl, N(C₁₋₆ alkyl)₂, N₃, S(O)C₁₋₆ alkyl, S(O)₂C₁₋₆ alkyl, NHSO₂C₁₋₆ alkyl, C(O)C₁₋₆ alkyl, C(O)OC₁₋₆ alkyl, NHC(O)C₁₋₆ alkyl, N(C₁₋₆ alkyl)C(O)C₁₋₆ alkyl, C(O)NHC₁₋₆ alkyl, C(O)N(C₁₋₆ alkyl)₂, CH(NH₂)C₁₋₆alkyl, CH(N[C₁₋₆ alkyl]₂)C₁₋₆alkyl, CH(NH₂)C₁₋₆alkylene-O—C₁₋₆alkyl, CH(N[C₁₋₆ alkyl]₂)C₁₋₆alkylene-O—C₁₋₆alkyl, C₃₋₆ cycloalkyl, 5-7 membered heterocycle, C₆₋₁₀ aryl, and C₅₋₁₀ heteroaryl;
R⁴ is (CH₂)n₄R⁴⁰;
n4 is 0, 1, 2, or 3;
R⁴⁰ is selected from the group consisting of hydrogen, C₁₋₆ alkyl, C₂₋₆ alkenyl, C₂₋₆ alkynyl, OH, C₁₋₆ alkoxy, halogen, C₁₋₆ haloalkyl, NO₂, CN, NH₂, NHC₁₋₆ alkyl, N(C₁₋₆ alkyl)₂, N₃, S(O)C₁₋₆ alkyl, S(O)₂C₁₋₆ alkyl, NHSO₂C₁₋₆ alkyl, C(O)C₁₋₆ alkyl, C(O)OC₁₋₆ alkyl, NHC(O)C₁₋₆ alkyl, N(C₁₋₆ alkyl)C(O)C₁₋₆ alkyl, C(O)NHC₁₋₆ alkyl, C(O)N(C₁₋₆ alkyl)₂, CH(NH₂)C₁₋₆alkyl, CH(N[C₁₋₆ alkyl]₂)C₁₋₆alkyl, CH(NH₂)C₁₋₆alkylene-O—C₁₋₆alkyl, CH(N[C₁₋₆ alkyl]₂)C₁₋₆alkylene-O—C₁₋₆alkyl, C₃₋₆ cycloalkyl, 5-7 membered heterocycle, C₆₋₁₀ aryl, and C₅₋₁₀ heteroaryl;
L¹ is
   a) divalent C₃ cycloalkyl;
   b) [(CRL)₂]m; or
   c) a bond
m is 0, 1, 2, or 3
each R^{L} independently is selected from the group consisting of hydrogen, C₁₋₆ alkyl, C₂₋₆ alkenyl, C₂₋₆ alkynyl, OH, C₁₋₆ alkoxy, halogen, C₁₋₆ haloalkyl, NO₂, CN, NH₂, NHC₁₋₆ alkyl, N(C₁₋₆ alkyl)₂, N₃, S(O)C₁₋₆ alkyl, S(O)₂C₁₋₆ alkyl, NHSO₂C₁₋₆ alkyl, C(O)C₁₋₆ alkyl, C(O)OC₁₋₆ alkyl, NHC(O)C₁₋₆ alkyl, C(O)NHC₁₋₆ alkyl, C₃₋₆ cycloalkyl, 5-7 membered heterocycle, C₆₋₁₀ aryl, and C₅₋₁₀ heteroaryl;
A² is
   a) a 5- or 6-membered heteroaryl ring which contains one, two, or three heteroatoms selected from O, N, or S; or
   b) a 6-membered aromatic ring, which may contain one heteroatom;
R³ is selected from the group consisting of hydrogen, C₁₋₆ alkyl, C₂₋₆ alkenyl, C₂₋₆ alkynyl, C₁₋₆ alkoxy, halogen, C₁₋₆ haloalkyl, NO₂, CN, NH₂, NHC₁₋₆ alkyl, N(C₁₋₆ alkyl)₂, N₃, S(O)C₁₋₆ alkyl, S(O)₂C₁₋₆ alkyl, NHSO₂C₁₋₆ alkyl, C(O)C₁₋₆ alkyl, C(O)OC₁₋₆ alkyl, NHC(O)C₁₋₆ alkyl, C(O)NHC₁₋₆ alkyl, C₃₋₆ cycloalkyl, 5-7 membered heterocycle, C₆₋₁₀ aryl, and C₅₋₁₀ heteroaryl,
wherein
each C₃₋₆ cycloalkyl, 5-7 membered heterocycle, C₆₋₁₀ aryl, and C₅₋₁₀ heteroaryl is unsubstituted or substituted with one, two, or three (CH₂)₀₋₃R^{S};
and
each R^{S} is selected from the group consisting of C₁₋₆ alkyl, C₂₋₆ alkenyl, C₂₋₆ alkynyl, OH, C₁₋₆ alkoxy, halogen, C₁₋₆ haloalkyl, NO₂, CN, NH₂, NHC₁₋₆ alkyl, N(C₁₋₆ alkyl)₂, N₃, S(O)C₁₋₆ alkyl, S(O)₂C₁₋₆ alkyl, NHSO₂C₁₋₆ alkyl, C(O)C₁₋₆ alkyl, C(O)OC₁₋₆ alkyl, NHC(O)C₁₋₆ alkyl, C(O)NHC₁₋₆ alkyl, unsubstituted C₃₋₆ cycloalkyl, O-unsubstituted C₃₋₆ cycloalkyl, unsubstituted 5-7 membered heterocycle, unsubstituted C₆₋₁₀ aryl, and unsubstituted C₅₋₁₀ heteroaryl.

In one aspect, R¹ is C₁₋₁₀ alkyl, C₂₋₁₀ alkynyl, C₁₋₁₀ alkoxy, or C₆ aryl, which is optionally substituted with C₁₋₆ alkyl, C₂₋₆ alkenyl, C₂₋₆ alkynyl, C₁₋₆ alkoxy, (CH₂)0-3C₃₋₆ cycloalkyl, or OC₃₋₆ cycloalkyl.

In one aspect, R¹ is C₁₋₁₀ alkoxy or C aryl, which is optionally substituted with C₁₋₆ alkyl, C₂₋₆ alkenyl, C₂₋₆ alkynyl, C₁₋₆ alkoxy, or (CH₂)₀₋₃C₃₋₆ cycloalkyl.

In one aspect, A¹ is phenyl.

In one aspect, X is CH. In one aspect, n2 is 1. In one aspect, R²⁰ is OH.

In one aspect, X is N. In one aspect, n2 is 1. In one aspect, R²⁰ is CH(NH₂)C₁₋₆alkyl, CH(N[C₁₋₆ alkyl]₂)C₁₋₆alkyl, CH(NH₂)C₁₋₆alkylene-O—C₁₋₆alkyl, or CH(N[C₁₋₆ alkyl]₂)C₁₋₆alkylene-O—C₁₋₆alkyl.

In one aspect, R⁴ is H or C₁₋₆ alkyl.

In one aspect, wherein L¹ is divalent C₃ cyclolakyl.

In one aspect, L¹ is [C(R^{L})₂]ₘ.

In one aspect, one R^{L} is H and one R^{L} is C₁₋₆ alkyl

In one aspect, A² is phenyl.

In one aspect, R³ is hydrogen or C₁₋₆ alkyl.

One embodiment of the present disclosure includes a compound or a pharmaceutically acceptable salt thereof selected from the group consisting of:

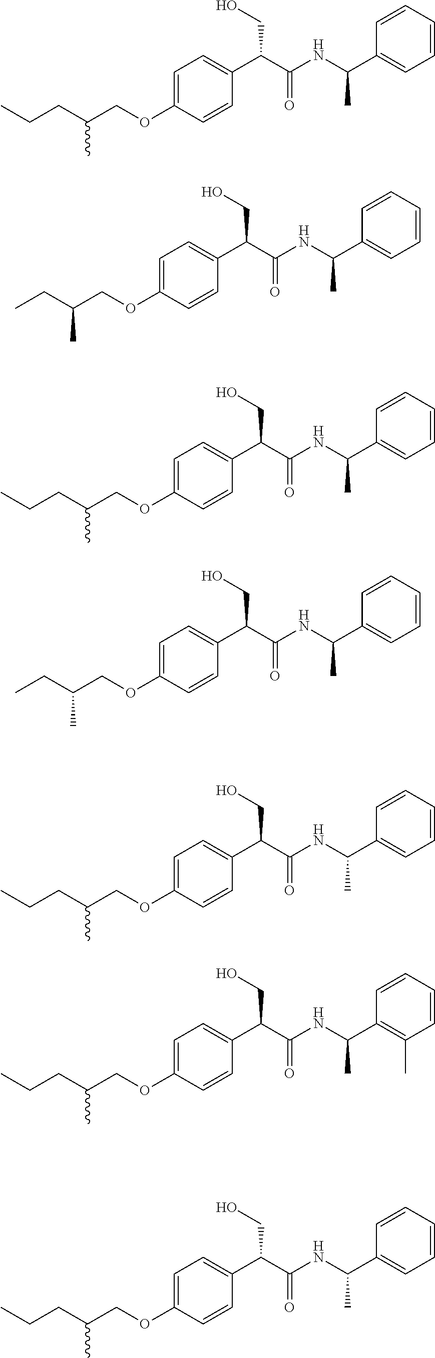

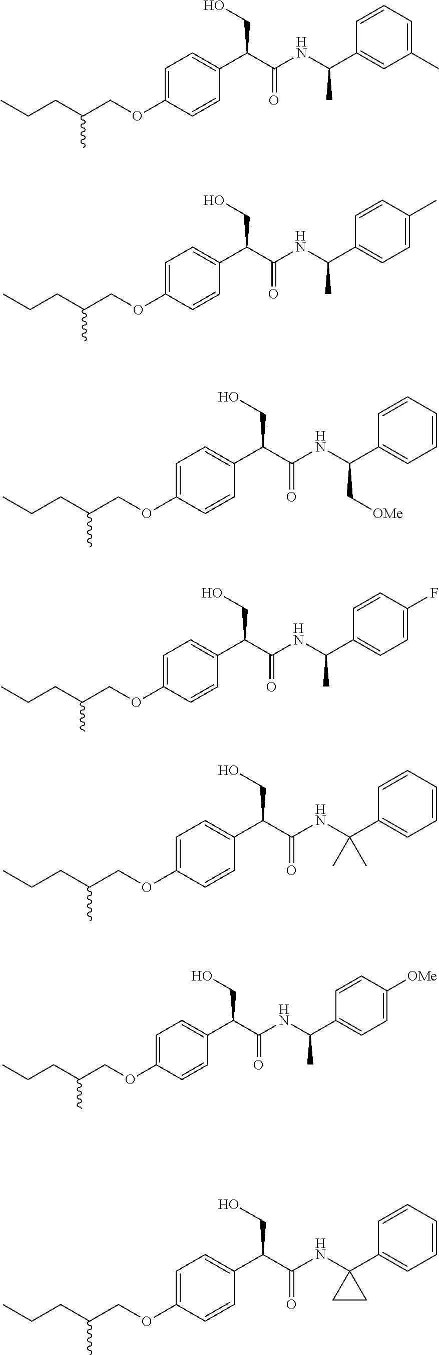

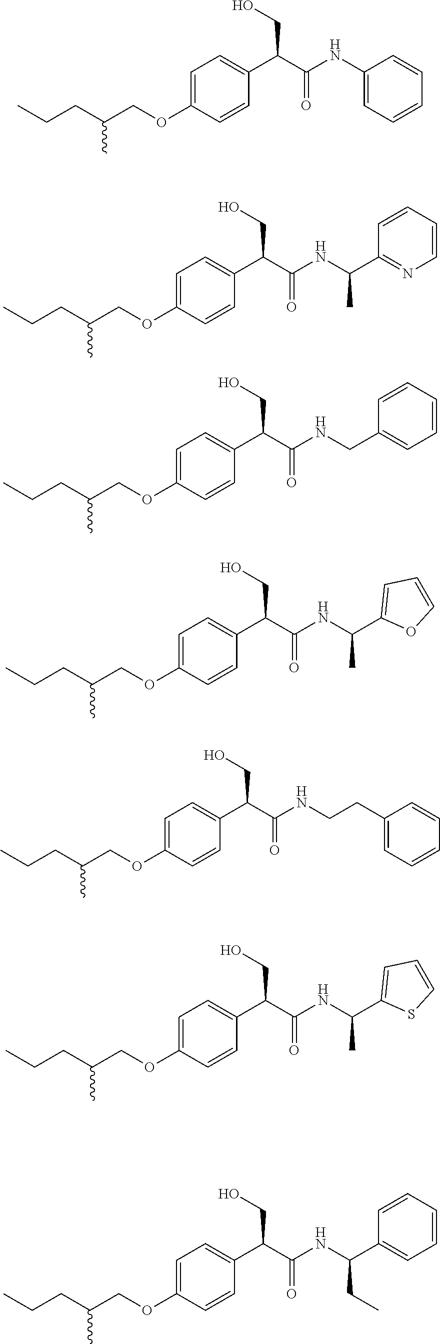

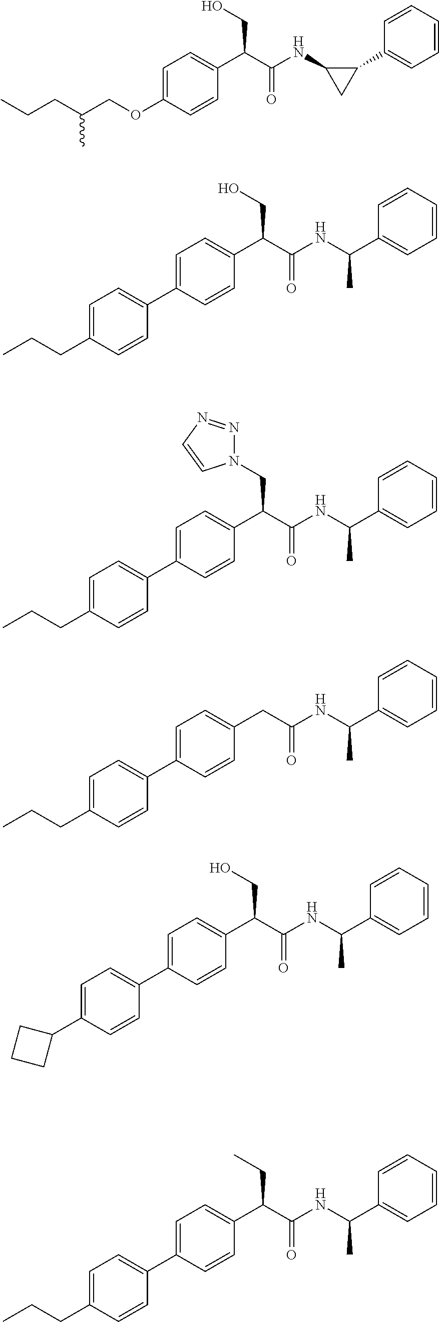 [SMILES: CCCC(C)COc1ccc([C@@H](CO)C(=O)N[C@@H]2C[C@H]2c2ccccc2)cc1 ; CCCc1ccc(-c2ccc([C@@H](CO)C(=O)N[C@H](C)c3ccccc3)cc2)cc1 ; CCCc1ccc(-c2ccc([C@@H](Cn3ccnn3)C(=O)N[C@H](C)c3ccccc3)cc2)cc1 ; CCCc1ccc(-c2ccc(CC(=O)N[C@H](C)c3ccccc3)cc2)cc1 ; C[C@@H](NC(=O)[C@H](CO)c1ccc(-c2ccc(C3CCC3)cc2)cc1)c1ccccc1 ; CCCc1ccc(-c2ccc([C@@H](CC)C(=O)N[C@H](C)c3ccccc3)cc2)cc1]

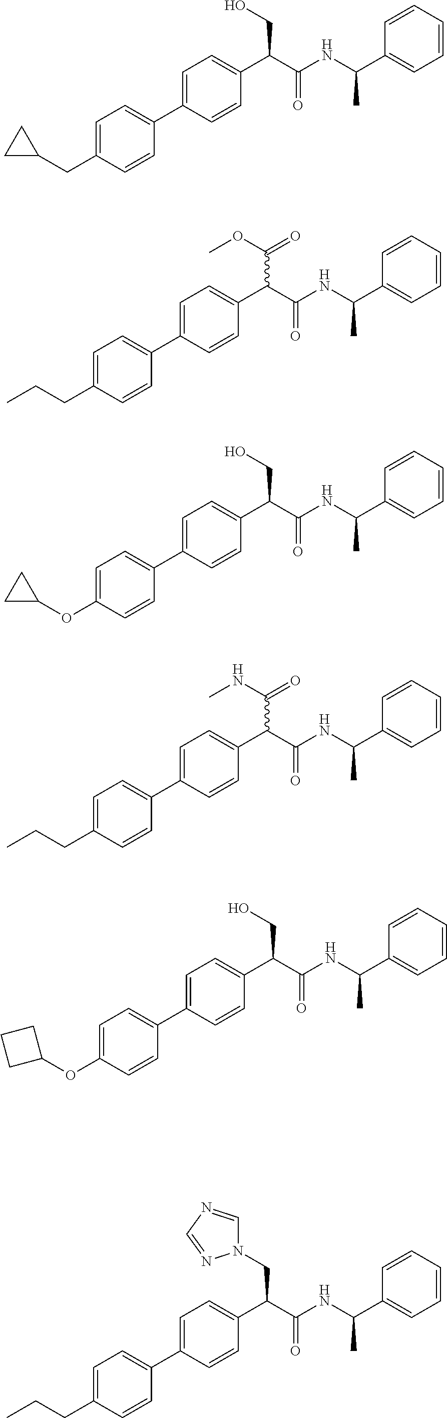 [SMILES: C[C@@H](NC(=O)[C@H](CO)c1ccc(-c2ccc(CC3CC3)cc2)cc1)c1ccccc1 ; CCCc1ccc(-c2ccc(C(C(=O)N[C@H](C)c3ccccc3)C(=O)OC)cc2)cc1 ; C[C@@H](NC(=O)[C@H](CO)c1ccc(-c2ccc(OC3CC3)cc2)cc1)c1ccccc1 ; CCCc1ccc(-c2ccc(C(C(=O)NC)C(=O)N[C@H](C)c3ccccc3)cc2)cc1 ; C[C@@H](NC(=O)[C@H](CO)c1ccc(-c2ccc(OC3CCC3)cc2)cc1)c1ccccc1 ; CCCc1ccc(-c2ccc([C@@H](Cn3cncn3)C(=O)N[C@H](C)c3ccccc3)cc2)cc1]

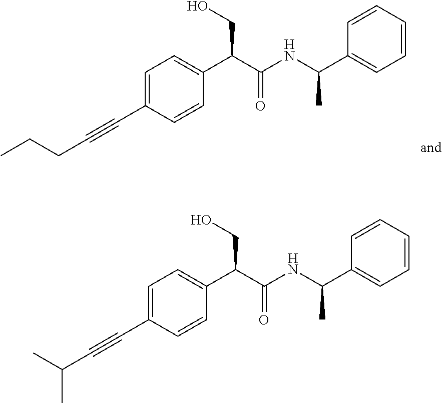 [SMILES: CCCC#Cc1ccc([C@@H](CO)C(=O)N[C@H](C)c2ccccc2)cc1 ; CC(C)C#Cc1ccc([C@@H](CO)C(=O)N[C@H](C)c2ccccc2)cc1]

One embodiment of the present disclosure includes a compound or a pharmaceutically acceptable salt thereof selected from the group consisting of:

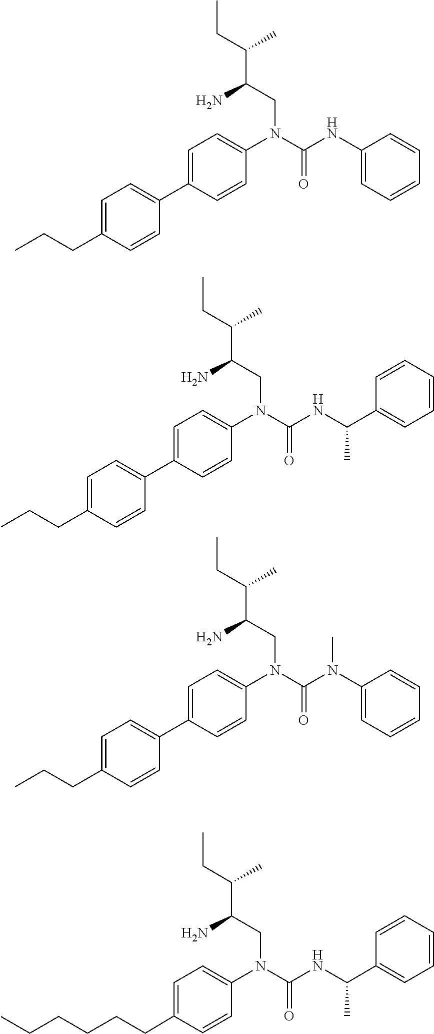 [SMILES: CCCc1ccc(-c2ccc(N(C[C@@H](N)[C@@H](C)CC)C(=O)Nc3ccccc3)cc2)cc1 ; CCCc1ccc(-c2ccc(N(C[C@@H](N)[C@@H](C)CC)C(=O)N[C@@H](C)c3ccccc3)cc2)cc1 ; CCCc1ccc(-c2ccc(N(C[C@@H](N)[C@@H](C)CC)C(=O)N(C)c3ccccc3)cc2)cc1 ; CCCCCCc1ccc(N(C[C@@H](N)[C@@H](C)CC)C(=O)N[C@@H](C)c2ccccc2)cc1]

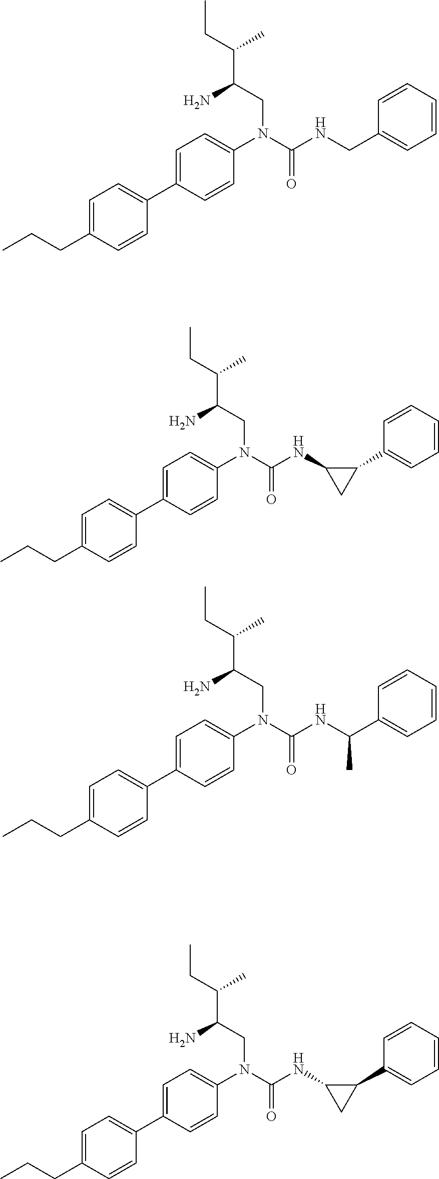 [SMILES: CCCc1ccc(-c2ccc(N(C[C@@H](N)[C@@H](C)CC)C(=O)NCc3ccccc3)cc2)cc1 ; CCCc1ccc(-c2ccc(N(C[C@@H](N)[C@@H](C)CC)C(=O)N[C@@H]3C[C@H]3c3ccccc3)cc2)cc1 ; CCCc1ccc(-c2ccc(N(C[C@@H](N)[C@@H](C)CC)C(=O)N[C@H](C)c3ccccc3)cc2)cc1 ; CCCc1ccc(-c2ccc(N(C[C@@H](N)[C@@H](C)CC)C(=O)N[C@H]3C[C@@H]3c3ccccc3)cc2)cc1]

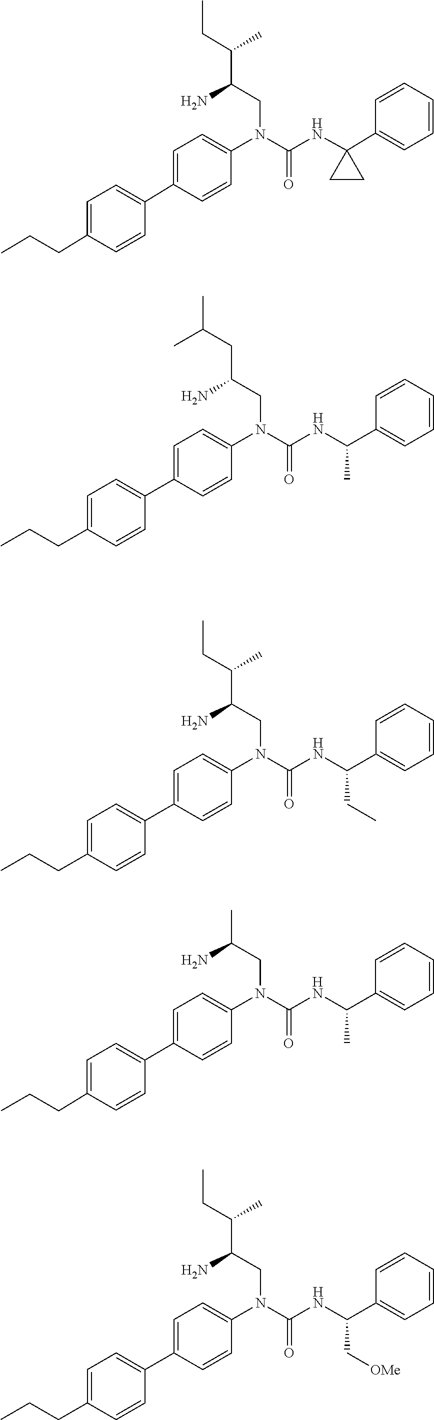 [SMILES: CCCc1ccc(-c2ccc(N(C[C@@H](N)[C@@H](C)CC)C(=O)NC3(c4ccccc4)CC3)cc2)cc1 ; CCCc1ccc(-c2ccc(N(C[C@H](N)CC(C)C)C(=O)N[C@@H](C)c3ccccc3)cc2)cc1 ; CCCc1ccc(-c2ccc(N(C[C@@H](N)[C@@H](C)CC)C(=O)N[C@@H](CC)c3ccccc3)cc2)cc1 ; CCCc1ccc(-c2ccc(N(C[C@H](C)N)C(=O)N[C@@H](C)c3ccccc3)cc2)cc1 ; CCCc1ccc(-c2ccc(N(C[C@@H](N)[C@@H](C)CC)C(=O)N[C@@H](COC)c3ccccc3)cc2)cc1]

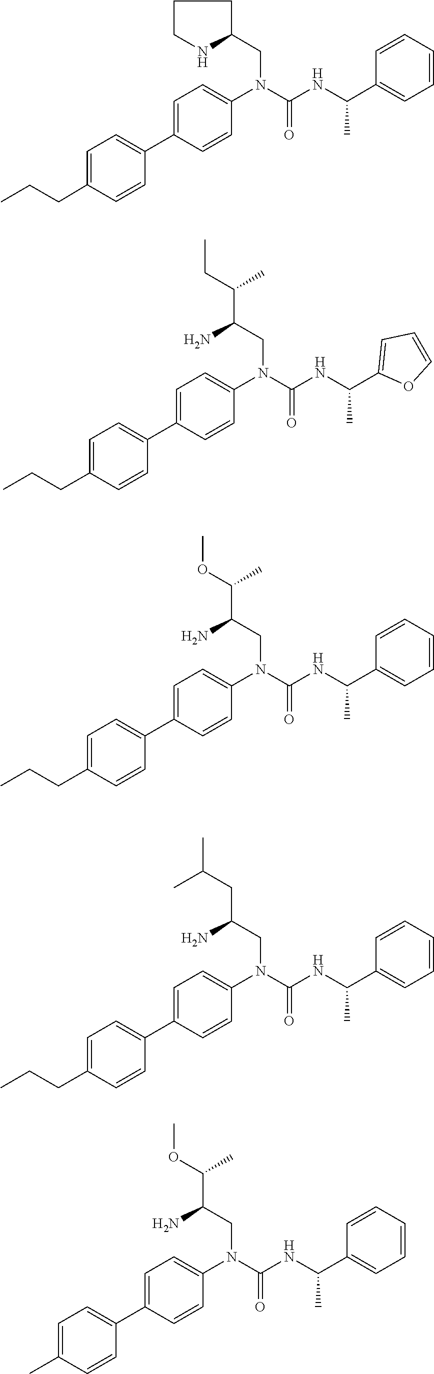 [SMILES: CCCc1ccc(-c2ccc(N(C[C@@H]3CCCN3)C(=O)N[C@@H](C)c3ccccc3)cc2)cc1 ; CCCc1ccc(-c2ccc(N(C[C@@H](N)[C@@H](C)CC)C(=O)N[C@@H](C)c3ccco3)cc2)cc1 ; CCCc1ccc(-c2ccc(N(C[C@@H](N)[C@@H](C)OC)C(=O)N[C@@H](C)c3ccccc3)cc2)cc1 ; CCCc1ccc(-c2ccc(N(C[C@@H](N)CC(C)C)C(=O)N[C@@H](C)c3ccccc3)cc2)cc1 ; CO[C@H](C)[C@H](N)CN(C(=O)N[C@@H](C)c1ccccc1)c1ccc(-c2ccc(C)cc2)cc1]

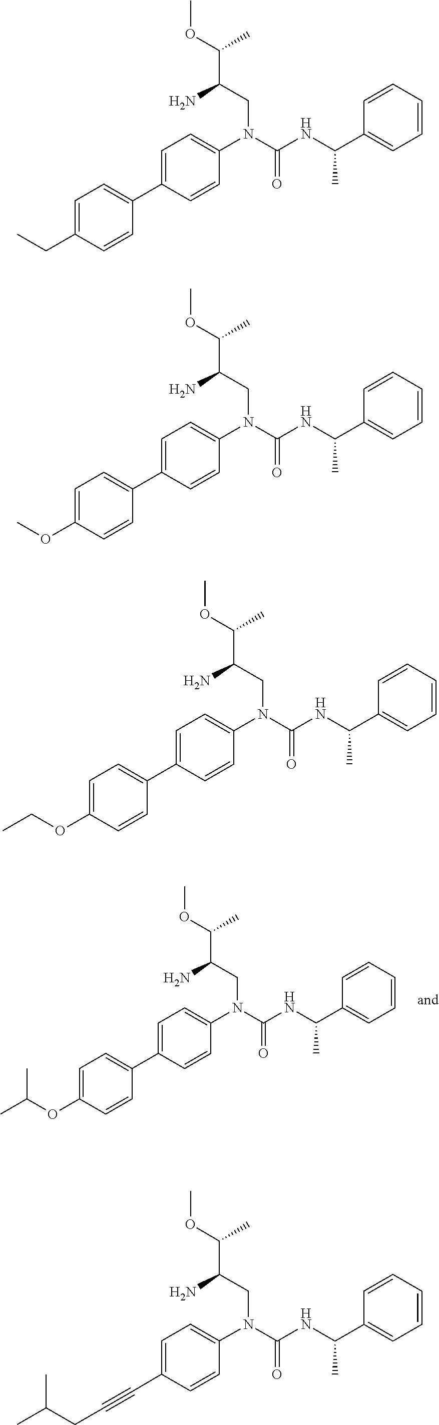 [SMILES: CCc1ccc(-c2ccc(N(C[C@@H](N)[C@@H](C)OC)C(=O)N[C@@H](C)c3ccccc3)cc2)cc1 ; COc1ccc(-c2ccc(N(C[C@@H](N)[C@@H](C)OC)C(=O)N[C@@H](C)c3ccccc3)cc2)cc1 ; CCOc1ccc(-c2ccc(N(C[C@@H](N)[C@@H](C)OC)C(=O)N[C@@H](C)c3ccccc3)cc2)cc1 ; CO[C@H](C)[C@H](N)CN(C(=O)N[C@@H](C)c1ccccc1)c1ccc(-c2ccc(OC(C)C)cc2)cc1 ; CO[C@H](C)[C@H](N)CN(C(=O)N[C@@H](C)c1ccccc1)c1ccc(C#CCC(C)C)cc1]

One embodiment of the present disclosure includes a method of treating a disease of disorder in a patient in needed thereof where modulation of the G protein-coupled receptor is beneficial comprising administering a compound of the present disclosure.

One embodiment of the present disclosure includes use of a compound of the present disclosure in the manufacture of a medicament for the treatment of a disease or disorder disease of disorder where modulation of the G protein-coupled receptor is beneficial, for a patient in needed thereof.

One embodiment of the present disclosure includes a compound of the present disclosure for use in the treatment of a disease of disorder where modulation of the G protein-coupled receptor is beneficial, for a patient in needed thereof.

In one aspect, the method, use, or compound for use provides treatment for a neurological disorder. In one aspect, the method, use, or compound for use includes wherein the neurological disorder is selected from one or more of psychosis, cognitive deficits in schizophrenia, affective disorders, attention deficit hyperactivity disorders, bipolar disorder, drug addiction, alcohol addiction, food addiction, activity addiction, Parkinson's disease, and Alzheimer's disease. In one aspect, the method, use, or compound for use of provides treatment for a metabolic disease. In one aspect, the method, use, or compound for use includes wherein the metabolic disease is selected from one or more of obesity and diabetes. In one aspect, the method, use, or compound for use provides treatment for an alcohol use disorder.

In one aspect, the compounds of the present disclosure may be used as GPR88 receptor agonists and may exhibit greater potency and experience reduced side effects, resulting in improved efficacy, pharmacokinetics, and safety. The compounds are believed useful for the treatment of diseases and conditions caused by agonism of the GPR88 receptor.

The scope of the present disclosure includes all combinations of aspects, embodiments, and preferences herein described.

### DETAILED DESCRIPTION OF THE DISCLOSURE

The following definitions are meant to clarify, but not limit, the terms defined. If a particular term used herein is not specifically defined, such term should not be considered indefinite. Rather, terms are used within their accepted meanings.

As used throughout this specification, the preferred number of atoms, such as carbon atoms, will be represented by, for example, the phrase “C, alkyl,” which refers to an alkyl group, as herein defined, containing the specified number of carbon atoms. Similar terminology will apply for other preferred terms and ranges as well. Thus, for example, C₁₋₄ alkyl represents a straight or branched chain hydrocarbon containing one to four carbon atoms.

As used herein, the term “alkyl” alone or in combination with any other term, refers to a straight or branched chain hydrocarbon. Examples of “alkyl” as used herein include, but are not limited to, methyl, ethyl, propyl, isopropyl, isobutyl, n-butyl, tert-butyl, sec-butyl, iso-pentyl, n-pentyl, n-hexyl, and the like.

As used herein, the term “alkenyl” refers to a straight or branched chain aliphatic hydrocarbon containing one or more carbon-to-carbon double bonds, and which may be optionally substituted, with multiple degrees of substitution being allowed. Examples of “alkenyl” as used herein include, but are not limited to, vinyl, and allyl.

As used herein, the term “alkoxy” refers to a group O-alkyl.

As used herein, the term “alkylene” refers to an optionally substituted straight divalent hydrocarbon radical. Examples of “alkylene” as used herein include, but are not limited to, methylene, ethylene, n-propylene, n-butylene, and the like.

As used herein the term “alkynyl” refers to a straight or branched chain aliphatic hydrocarbon containing one or more carbon-to-carbon triple bonds, which may be optionally substituted, with multiple degrees of substitution being allowed. An example of “alkynyl” as used herein includes, but is not limited to, ethynyl.

As used herein, the term “aryl” refers to a single benzene ring, fused, bridged, or spirocyclic benzene ring system which may be optionally substituted, with multiple degrees of substitution being allowed. Examples of “aryl” groups as used include, but are not limited to, phenyl, 2-naphthyl, 1-naphthyl, anthracene, and phenanthrene. Preferable aryl rings have five-to ten-members.

As used herein, a fused benzene ring system encompassed within the term “aryl” includes fused polycyclic hydrocarbons, namely where a cyclic hydrocarbon with less than maximum number of noncumulative double bonds, for example where a saturated hydrocarbon ring (cycloalkyl, such as a cyclopentyl ring) is fused with anaromatic ring (aryl, such as a benzene ring) to form, for example, groups such as indanyl and acenaphthalenyl, and also includes such groups as, for non-limiting examples, dihydronaphthalene and tetrahydronaphthalene.

As used herein, the term “cycloalkyl” refers to a fully saturated optionally substituted monocyclic, bicyclic, bridged, or spirocyclic hydrocarbon ring, with multiple degrees of substitution being allowed. Exemplary “cycloalkyl” groups as used herein include, but are not limited to, cyclopropyl, cyclobutyl, cyclopentyl, cyclohexyl, and cycloheptyl.

As used herein the term “halogen” refers to fluorine, chlorine, bromine, or iodine.

As used herein the term “haloalkyl” refers to an alkyl group, as defined herein, which is substituted with at least one halogen. Examples of branched or straight chained “haloalkyl” groups as used herein include, but are not limited to, methyl, ethyl, propyl, isopropyl, n-butyl, and t-butyl substituted independently with one or more halogens, for example, fluoro, chloro, bromo, and iodo. The term “haloalkyl” should be interpreted to include such substituents as perfluoroalkyl groups such as CF₃.

As used herein, the term “heteroaryl” refers to a monocyclic five to seven membered aromatic ring, or to a fused, bridged, or spirocyclic aromatic ring system comprising two or more of such rings, which may be optionally substituted, with multiple degrees of substitution being allowed. Preferably, such rings contain five- to ten-members. These heteroaryl rings contain one or more nitrogen, sulfur, and/or oxygen atoms, where N-oxides, sulfur oxides, and dioxides are permissible heteroatom substitutions. Examples of “heteroaryl” groups as used herein include, but are not limited to, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzoxazole, benzothiophene, indole, indazole, benzimidazole, imidazopyridine, pyrazolopyridine, and pyrazolopyrimidine.

As used herein, the terms “heterocycle” and “heterocyclyl” refers to a fully saturated optionally substituted monocyclic, bicyclic, bridged, or spirocyclic hydrocarbon ring, with multiple degrees of substitution being allowed, which contains one or more heteroatom selected from nitrogen, sulfur, and/or oxygen atoms, where N-oxides, sulfur oxides, and dioxides are permissible. Exemplary “heterocyclyl” groups as used herein include, but are not limited to, azetidine, pyrrolidinyl, piperidinyl, piperazinyl, hexahydroazepine, and morpholinyl.

Typically, but not absolutely, the salts of the present disclosure are pharmaceutically acceptable salts. Salts encompassed within the term “pharmaceutically acceptable salts” refer to non-toxic salts of the compounds of this disclosure. Salts of the compound of the present disclosure may comprise acid addition salts. Representative salts include acetate, benzenesulfonate, benzoate, bicarbonate, bisulfate, bitartrate, borate, calcium edetate, camsylate, carbonate, clavulanate, citrate, dihydrochloride, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, sulfate, tannate, tartrate, teoclate, tosylate, triethiodide, trimethylammonium, and valerate salts. Other salts, which are not pharmaceutically acceptable, may be useful in the preparation of compounds of this disclosure and these should be considered to form a further aspect of the disclosure.

The compounds of formula (I) may crystallize in more than one form, a characteristic known as polymorphism, and such polymorphic forms (“polymorphs”) are within the scope of formula (I). Polymorphism generally can occur as a response to changes in temperature, pressure, or both. Polymorphism can also result from variations in the crystallization process. Polymorphs can be distinguished by various physical characteristics known in the art such as x-ray diffraction patterns, solubility, and melting point.

As used herein, the term “effective amount” means that amount of a drug or pharmaceutical agent that will elicit the biological or medical response of a tissue, system, animal, or human that is being sought, for instance, by a researcher or clinician. The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. Theterm also includes within its scope amounts effective to enhance normal physiological function.

For use in therapy, therapeutically effective amounts of a compound of formula (I), as well as salts or solvates thereof, may be administered as the raw chemical. Additionally, the active ingredient may be presented as a pharmaceutical composition.

Accordingly, the disclosure further provides pharmaceutical compositions that include effective amounts of one or more compounds of the formula (I), or a salt or solvate thereof, and one or more pharmaceutically acceptable carriers, diluents, or excipients. The compound of formula (I) or a salt or solvate thereof, are as herein described. The carrier(s), diluent(s), or excipient(s) must be acceptable, in the sense of being compatible with the other ingredients of the formulation and not deleterious to the recipient of the pharmaceutical composition.

GPR88 is a striatal-enriched orphan G protein-coupled receptor (GPCR) whose expression varies over development in the brain of rodents, monkeys, and humans. Gene association studies in humans have uncovered a link between GPR88 function and several psychiatric, neurodevelopmental, or neurodegenerative disorders, including schizophrenia, bipolar disorder, speech delay, and chorea. In rodents, transcript levels of Gpr8B gene were found modified following exposure to various psychoactive drugs, such as mood stabilizers, antidepressants, methamphetamine, L-DOPA, and drugs of abuse. GPR88 is a promising target for the development of innovative treatments for CNS pathologies. In mice, deletion of the Gpr88 gene alters primarily striatal physiology, striatum-centered brain networks and striatal-dependent behaviors, with notably severe deficits in motor coordination and skill learning, hyperactivity, stereotypies and altered reward-driven behaviors. GPR88 function, however, extends beyond striatal-mediated responses, in accordance with extra-striatal GPR88 expression and widespread modifications in brain connectivity, gene expression and behavioral responses in Gpr88 null (Gpr8B^{-/-}) mice. Thus, GPR88 has a major influence on brain physiology and controls a vast repertoire of behaviors.

The compounds of the present disclosure are believed useful in the treatment of psychiatric, neurodevelopmental, or neurodegenerative disorders. GPR88 modulation effects the regulation of various brain and behavioral functions, including cognition, mood, movement control, cue-based reward learning, and basal ganglia associated disorders. The compounds of the present disclosure are believed useful in the treatment of CNS disorders including schizophrenia, Parkinson's disease, anxiety, and addiction. In particular, the compounds of the present disclosure are believed useful in the treatment of alcohol use disorder (AUD).

The compounds of the present disclosure may be used for any disease of disorder where modulation of the G protein-coupled receptor is beneficial. In one embodiment, the compounds of the present disclosure may be used to treat a neurological disorder. In one aspect, the neurological disorder is selected from one or more of psychosis, cognitive deficits in schizophrenia, affective disorders, attention deficit hyperactivity disorders, bipolar disorder, drug addiction, alcohol addiction, food addiction, activity addiction, Parkinson's disease, and Alzheimer's disease. In another embodiment, the compounds of the present invention may be used to treat a metabolic disease. In one aspect, the metabolic disease is selected from one or more of obesity and diabetes.

The therapeutically effective amount of a compound of the present disclosure will depend upon a number of factors. For example, the species, age, and weight of the recipient, the precise condition requiring treatment and its severity, the nature of the formulation, and the route of administration are all factors to be considered. The therapeutically effective amount ultimately should be at the discretion of the attendantphysician or veterinarian. Regardless, an effective amount of a compound of formula

(I) for the treatment of humans suffering from frailty, generally, should be in the range of 0.1 to 100 mg/kg body weight of recipient (mammal) per day. More usually the effective amount should be in the range of 0.1 to 20 mg/kg body weight per day. Thus, for a 70 kg adult mammal one example of an actual amount per day would usually be from 10 to 2000 mg. This amount may be given in a single dose per day or in a number (such as two, three, four, five, or more) of sub-doses per day such that the total daily dose is the same. An effective amount of a salt or solvate thereof, may be determined as a proportion of the effective amount of the compound of formula (I) per se. Similar dosages should be appropriate for treatment of the other conditions referred to herein. Pharmaceutical formulations may be presented in unit dose forms containing a predetermined amount of active ingredient per unit dose. Such a unit may contain, as a non-limiting example, 1 mg to 2 g of a compound of the formula (I), depending on the condition being treated, the route of administration, and the age, weight, and condition of the patient. Preferred unit dosage formulations are those containing a daily dose or sub-dose, as herein above recited, or an appropriate fraction thereof, of an active ingredient. Such pharmaceutical formulations may be prepared by any of the methods well known in the pharmacy art.

Pharmaceutical formulations may be adapted for administration by any appropriate route, for example by an oral (including buccal or sublingual), rectal, nasal, topical (including buccal, sublingual or transdermal), vaginal, or parenteral (including subcutaneous, intramuscular, intravenous or intradermal) route. Such formulations may be prepared by any method known in the art of pharmacy, for example by bringing into association the active ingredient with the carrier(s) or excipient(s). By way of example, and not meant to limit the disclosure, with regard to certain conditions and disorders for which the compounds of the present disclosure are believed useful certain routes will be preferable to others. In addition, pharmaceutical formulations may be used to allow delayed or extended exposure to compound of formula (I) under circumstances where delayed or extended exposure would improve therapy.

Pharmaceutical formulations adapted for oral administration may be presented as discrete units such as capsules or tablets; powders or granules; solutions or suspensions, each with aqueous or non-aqueous liquids; edible foams or whips; or oil-in-water liquid emulsions or water-in-oil liquid emulsions. For instance, for oral administration in the form of a tablet or capsule, the active drug component can be combined with an oral, non-toxic pharmaceutically acceptable inert carrier such as ethanol, glycerol, water, and the like. Generally, powders are prepared by comminuting the compound to a suitable fine size and mixing with an appropriate pharmaceutical carrier such as an edible carbohydrate, as, for example, starch or mannitol. Flavorings, preservatives, dispersing agents, and coloring agents can also be present.

Capsules are made by preparing a powder, liquid, or suspension mixture and encapsulating with gelatin or some other appropriate shell material. Glidants and lubricants such as colloidal silica, talc, magnesium stearate, calcium stearate, or solid polyethylene glycol can be added to the mixture before the encapsulation. A disintegrating or solubilizing agent such as agar-agar, calcium carbonate or sodium carbonate can also be added to improve the availability of the medicament when the capsule is ingested. Moreover, when desired or necessary, suitable binders, lubricants, disintegrating agents, and coloring agents can also be incorporated into the mixture. Examples of suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants useful in these dosage forms include, for example, sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

Tablets are formulated, for example, by preparing a powder mixture, granulating or slugging, adding a lubricant and disintegrant, and pressing into tablets. A powder mixture may be prepared by mixing the compound, suitably comminuted, with a diluent or base as described above. Optional ingredients include binders such as carboxymethylcellulose, aliginates, gelatins, or polyvinyl pyrrolidone, solution retardants such as paraffin, resorption accelerators such as a quaternary salt, and/or absorption agents such as bentonite, kaolin, or dicalcium phosphate. The powder mixture can be wet-granulated with a binder such as syrup, starch paste, acadia mucilage or solutions of cellulosic or polymeric materials, and forcing through a screen. As an alternative to granulating, the powder mixture can be run through the tablet machine and the result is imperfectly formed slugs broken into granules. The granules can be lubricated to prevent sticking to the tablet-forming dies by means of the addition of stearic acid, a stearate salt, talc or mineral oil. The lubricated mixture is then compressed into tablets. The compounds of the present disclosure can also be combined with a free flowing inert carrier and compressed into tablets directly without going through the granulating or slugging steps. A clear or opaque protective coating consisting of a sealing coat of shellac, a coating of sugar or polymeric material, and a polish coating of wax can be provided. Dyestuffs can be added to these coatings to distinguish different unit dosages.

Oral fluids such as solutions, syrups, and elixirs can be prepared in dosage unit form so that a given quantity contains a predetermined amount of the compound. Syrups can be prepared, for example, by dissolving the compound in a suitably flavored aqueous solution, while elixirs are prepared through the use of a non-toxic alcoholic vehicle. Suspensions can be formulated generally by dispersing the compound in a non-toxic vehicle. Solubilizers and emulsifiers such as ethoxylated isostearyl alcohols and polyoxy ethylene sorbitol ethers, preservatives; flavor additives such as peppermint oil, or natural sweeteners, saccharin, or other artificial sweeteners; and the like can also be added.

Where appropriate, dosage unit formulations for oral administration can be microencapsulated. The formulation can also be prepared to prolong or sustain the release as for example by coating or embedding particulate material in polymers, wax or the like.

Pharmaceutical formulations adapted for topical administration in the mouth include lozenges, pastilles, and mouthwashes.

A compound of the present disclosure or a salt or solvate thereof, may be employed alone or in combination with other therapeutic agents. The compound of formula (I) and the other pharmaceutically active agent(s) may be administered together or separately and, when administered separately, administration may occur simultaneously or sequentially, in any order. The amounts of the compound of formula (I) and the other pharmaceutically active agent(s) and the relative timings of administration will be selected in order to achieve the desired combined therapeutic effect. The administration in combination of a compound of formula (I) or a salt or solvate thereof with other treatment agents may be in combination by administration concomitantly in: (1) a unitary pharmaceutical composition including a combination of compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time.

The compounds of this disclosure may be made by a variety of methods, including well-known standard synthetic methods. Illustrative general synthetic methods are set out below and then specific compounds of the disclosure are prepared in the working Examples.

In all of the examples described below, protecting groups for sensitive or reactive groups are employed where necessary in accordance with general principles of synthetic chemistry. Protecting groups are manipulated according to standard methods of organic synthesis (T. W. Green and P. G. M. Wuts (1999) Protecting Groups in Organic Synthesis, 3^{rd} Edition, John Wiley & Sons, incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of the present disclosure.

The present disclosure also provides a method for the synthesis of compounds of formula (I) and novel compounds useful as synthetic intermediates in the preparation of compounds of the present disclosure.

The compounds can be prepared according to the methods described below using readily available starting materials and reagents. In these reactions, variants may be employed which are themselves known to those of ordinary skill in this art, but are not mentioned in greater detail.

Unless otherwise stated, structures depicted herein are also meant to include compounds which differ only in the presence of one or more isotopically enriched atoms. Compounds having the present structure except for the replacement of a hydrogen atom by a deuterium or tritium, or the replacement of a carbon atom by a ¹³C- or ¹⁴C-enriched carbon are within the scope of the disclosure. For example, deuterium has been widely used to examine the pharmacokinetics and metabolism of biologically active compounds. Although deuterium behaves similarly to hydrogen from a chemical perspective, there are significant differences in bond energies and bond lengths between a deuterium-carbon bond and a hydrogen-carbon bond. Consequently, replacement of hydrogen by deuterium in a biologically active compound may result in a compound that generally retains its biochemical potency and selectivity but manifests significantly different absorption, distribution, metabolism, and/or excretion (ADME) properties compared to its isotope-free counterpart. Thus, deuterium substitution may result in improved drug efficacy, safety, and/or tolerability for some biologically active compounds.

In accordance with another aspect of the disclosure there is also provided a process for the preparation of a pharmaceutical formulation including admixing a compound of the formula (I) or salts, solvates, and physiological functional derivatives thereof, with one or more pharmaceutically acceptable carriers, diluentsor excipients.

Those skilled in the art of organic synthesis will appreciate that there exist multiple means of producing compounds of the present disclosure which are labeled with a radioisotope appropriate to various uses.

### EXPERIMENTAL SECTION

### Abbreviations

As used herein the symbols and conventions used in these processes, schemes and examples are consistent with those used in the contemporary scientific literature, for example, the Journal of the American Chemical Society or the Journal of Biological Chemistry. Specifically, the following abbreviations may be used in the examples and throughout the specification:
g (grams); mg (milligrams);
L (liters); mL (milliliters);
μL (microliters); psi (pounds per square inch);
M (molar); mM (millimolar);
Hz (Hertz); MHz (megahertz);
mol (moles); mmol (millimoles); RT or rt (room temperature); hr (hours);
min (minutes); TLC (thin layer chromatography);
mp (melting point); RP (reverse phase);
Tᵣ (retention time); TFA (trifluoroacetic acid);
TEA (triethylamine); THE (tetrahydrofuran);
TFAA (trifluoroacetic anhydride); CD₃OD (deuterated methanol); CDCl₃ (deuterated
chloroform); DMSO (dimethylsulfoxide); SiO₂ (silica gel); atm
(atmosphere);
EtOAc (ethyl acetate); CHCl₃ (chloroform);
HCl (hydrochloric acid); Ac (acetyl); DMF (N,N-dimethylformamide); Me
(methyl); Cs₂CO₃ (cesium carbonate); EtOH (ethanol); Et (ethyl); t-Bu (tert-butyl);
MeOH (methanol) p-TsOH (p-toluenesulfonic acid);
DCM (dichloromethane) DCE (dichloroethane)
Et₂O (diethyl ether) K₂CO₃ (potassium carbonate); Na₂CO₃ (sodium
carbonate); i-PrOH (isopropyl alcohol) NaHCO₃ (sodium
bicarbonate); ACN (acetonitrile);
Pr (propyl); i-Pr (isopropyl);
PE (petroleum ether); Hex (hexanes);
H₂SO₄ (sulfuric acid); HCl (hydrochloric acid);
Et₃N (triethylamine); Na₂SO₄ (sodium sulfate); MTBE (methyl tert-butyl
ether); Boc (tert-butoxycarbonyl); DIPEA
(diisopropylethylamine); IPA (isopropanol);
HMDS (hexamethyldisilazane) NH₄Cl (ammonium chloride)
NH₄CO₃ (ammonium carbonate) MgSO₄ (magnesium sulfate)NH₄OH (ammonium hydroxide)

All solvents and chemicals were reagent grade. Unless otherwise mentioned,all reagents and solvents were purchased from commercial vendors and used as received. Flash column chromatography was carried out on a Teledyne ISCO CombiFlash Rf system using prepacked columns. Solvents used include hexane, ethyl acetate (EtOAc), dichloromethane, methanol, and chloroform/methanol/ammonium hydroxide (80:18:2) (CMA-80). Purity and characterization of compounds were established by a combination of HPLC, TLC, mass spectrometry, and NMR analyses. ¹H and ¹³C NMR spectra were recorded on aBruker Avance DPX-300 (300 MHz) spectrometer and were determined in chloroform-d, DMSO-d6, or methanol-d4 with tetramethylsilane (TMS) (0.00 ppm) or solvent peaks as the internal reference. Chemical shifts are reported in ppm relative to the reference signal, and coupling constant (J) values are reported in hertz (Hz).

Thin layer chromatography (TLC) was performed on EMD precoated silica gel 60 F₂₅₄ plates, and spots were visualized with UV light or iodine staining. Low resolution mass spectra were obtained using a Waters Alliance HT/Micromass ZQ system (ESI). All test compounds were greater than 95% pure as determined by HPLC on an Agilent 1100 system using an Agilent Zorbax SB-Phenyl, 2.1 mm×150 mm, 5 μm column with gradient elution using the mobile phases (A) H2O containing 0.1% CF₃COOH and (B) MeCN, with a flow rate of 1.0 mL/min.

All solvents and chemicals were reagent grade. Unless otherwise mentioned, all reagents and solvents were purchased from commercial vendors and used as received. Flash column chromatography was carried out on a Teledyne ISCO CombiFlash Rf system using prepacked columns. Solvents used include hexane, ethyl acetate (EtOAc), dichloromethane (DCM) and methanol. Purity and characterization of compounds were established by a combination of NMR, mass spectrometry, TLC, and HPLC analyses. ¹H and ¹³C NMR spectra were recorded on a Bruker Avance DPX-300 (300 MHz) spectrometer and were determined in CDCl₃, DMSO-d₆, or CD₃OD with tetramethylsilane (TMS) (0.00 ppm) or solvent peaks as the internal reference. Chemical shifts are reported in ppm relative to the reference signal and coupling constant (J) values are reported in hertz (Hz). Nominal mass spectra were obtained using an Agilent InfinityLab MSD single quadrupole mass spectrometer system (ESI). Thin layer chromatography (TLC) was performed on EMD precoated silica gel 60 F₂₅₄ plates, and spots were visualized with UV light or iodine staining. All final compounds were greater than 95% pure as determined by HPLC on a Waters 2695 Separation Module equipped with a Waters 2996 Photodiode Array Detector and a Phenomenex Synergi 4 mm Hydro-RP 80A C18 250×4.6 mm column using a flow rate of 1 mL/min starting with 1 min at 5% solvent B, followed by a 15 min gradient of 5-95% solvent B, followed by 9 min at 95% solvent B (solvent A, water with 0.1% TFA; solvent B, acetonitrile with 0.1% TFA and 5% water; absorbance monitored at 280 nm).

### Description of Chemistry and Biology

### Chemistry

Target compounds 3-6 were synthesized in accordance with reactions shown in Scheme A. O-Alkylation of p-iodophenol A1 with 2-methyl-1-pentanol under Mitsunobu conditions provided ether A2. Reaction of A2 with tert-butyl methyl malonate in the presence of NaH and CuBr furnished the malonate intermediate A3. Removal of the tert-butyl group with HCl led to the corresponding acid A4 which was subsequently subjected to acid chloride-mediated coupling with (R)-(+)-α-methylbenzylamine or 2-(1H-benzotriazol-1-yl)-1,1,3,3-tetramethyluronium hexafluorophosphate (HBTU)-mediated coupling with (S)-(−)-α-methylbenzylamine to give amides A5 and A6, respectively. Reduction of the ester functionality with freshly prepared LiBH₄ followed by chromatographic separation of the diastereomeric mixture provided pure alcohols 3-6. The absolute stereochemistry of 4 and 5 were confirmed by an asymmetric synthesis shown in Scheme B.

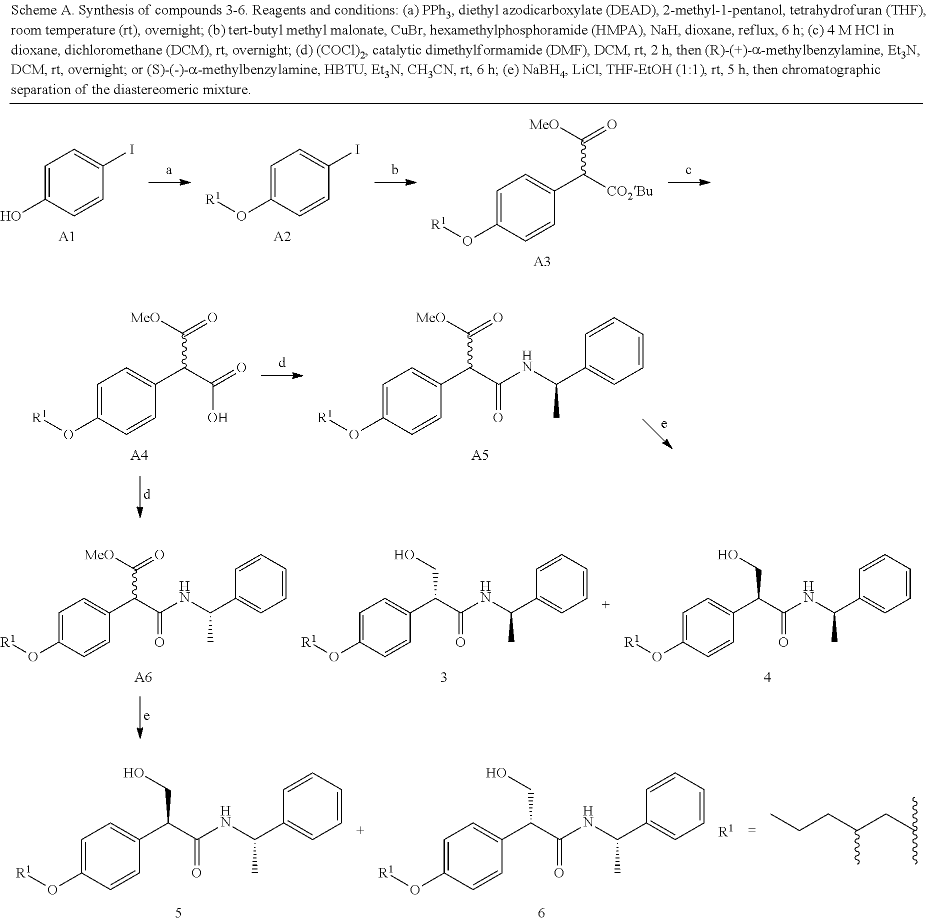

The asymmetric synthesis of amides 4, 5, and 7-24 were performed in accordance with reactions shown in Scheme B. Mitsunobu reaction of methyl 2-(4-hydroxyphenyl)acetate B1 with 2-methyl-1-pentanol afforded ether B12a. Saponification of the ester functionality with NaOH, followed by acidic workup gave the corresponding carboxylic acid (structure not shown), which was subsequently reacted with oxalyl chloride in dichloromethane (DCM) to provide acid chloride B3a. The acid chloride was coupled with (R)-4-benzyl-2-oxazolidinone in the presence of n-BuLi to afford amide B4a. The amide B4a was subjected to a TiCl₄ mediated asymmetric hydroxymethylation in the presence of s-trioxane to furnish alcohol B5a as the sole diastereomer. The oxazolidinone auxiliary was then removed by hydrolysis in the presence of H₂O₂ and LiOH to provide acid B6. Amides 4 and 5 were prepared by coupling of acid B6 with enantiomerically pure (R)-(+)-α-methylbenzylamine and (S)-(−)-α-methylbenzylamine, respectively, using N,N,N′,N′-tetramethylchloroformamidinium hexafluorophosphate (TCFH) and N-methylimidazole (NMI). Likewise, amides 9-24 were synthesized by coupling of B6 with appropriate amines as shown in Table 1. One the other hand, protection of the phenol group in B1 with triisopropylsilyl (TIPS) chloride afforded B2b, which was then converted to B5b by using the procedure, analogous to that used to prepare B5a. Protection of the methyl alcohol with a methoxyethoxymethyl (MEM) group led to B7, which was subjected to hydrolysis, followed by coupling of the resulting acid with R)-(+)-α-methylbenzylamine and removal of the TIPS protecting group, to provide phenol B8. Finally, alkylation of B8 with (2S)-2-methylbutyl 4-methylbenzene-1-sulfonate or (2R)-2-methylbutyl 4-methylbenzene-1-sulfonate, followed by MEM deprotection with ZnBr₂, furnished 7 and 8, respectively.

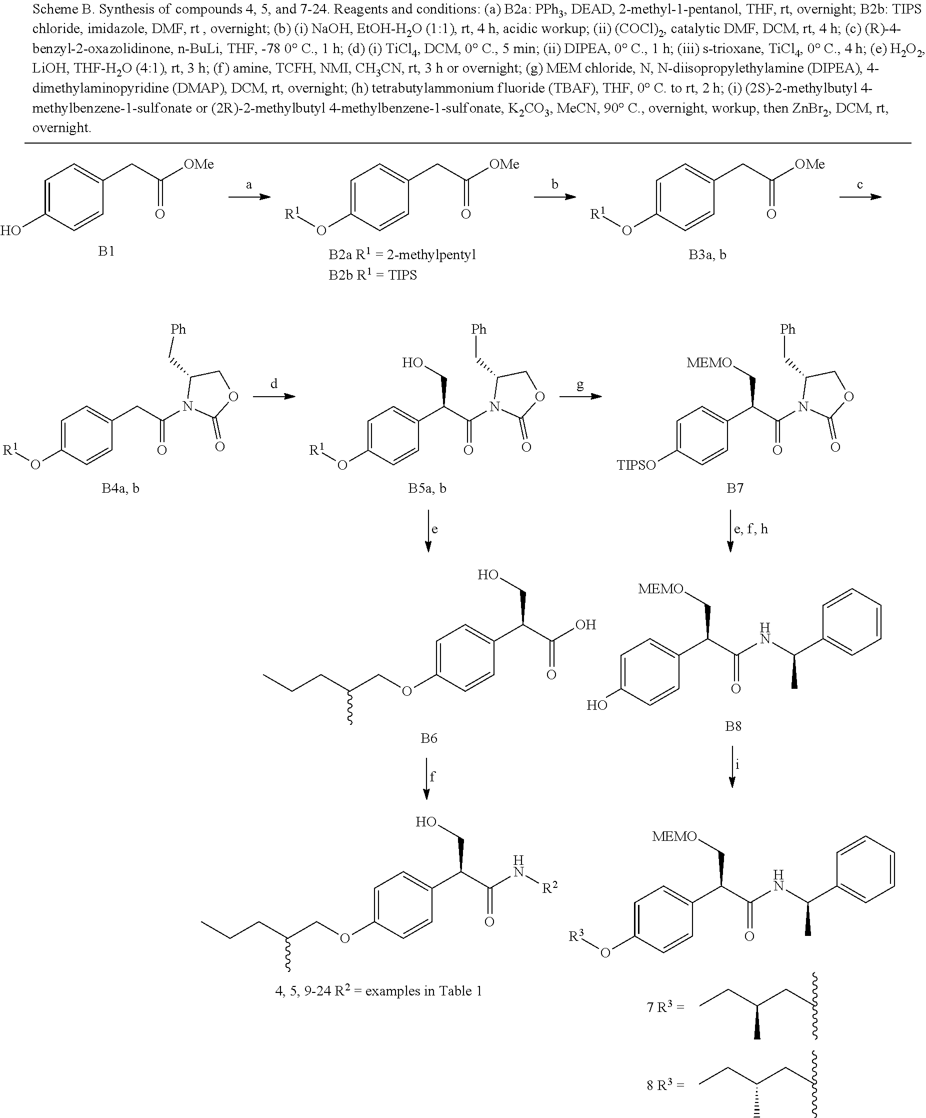

Target compounds 25-27 and 30-37 were synthesized in accordance with reactions shown in Scheme C. Conversion of acid chloride C1 to the 4-bromo phenylacetamide derivatives C5 was accomplished by using the procedure, analogous to that used to prepare 4 (Scheme B). Aryl bromide C5 (R⁴═OH or Me) was subjected to Suzuki coupling with 4-substituted phenylboronic acid to afford biphenyl analogs 25, 27, and 32-35. Similarly, C5 was subjected to Sonogashira coupling with alkyne to give the desired alkyne analogues 36 and 37. Reaction of acid chloride C1 with (R)-(+)-α-methylbenzylamine afforded amide C6, which was then coupled with 4-propylphenyl boronic acid to furnish 26. Lastly, alcohol 25 was treated with PPh₃ and CBr₄ to give bromide C7, which was subsequently reacted with 1,2,4-traizole or 1,2,3-triazole to yield 30 and 31, respectively.

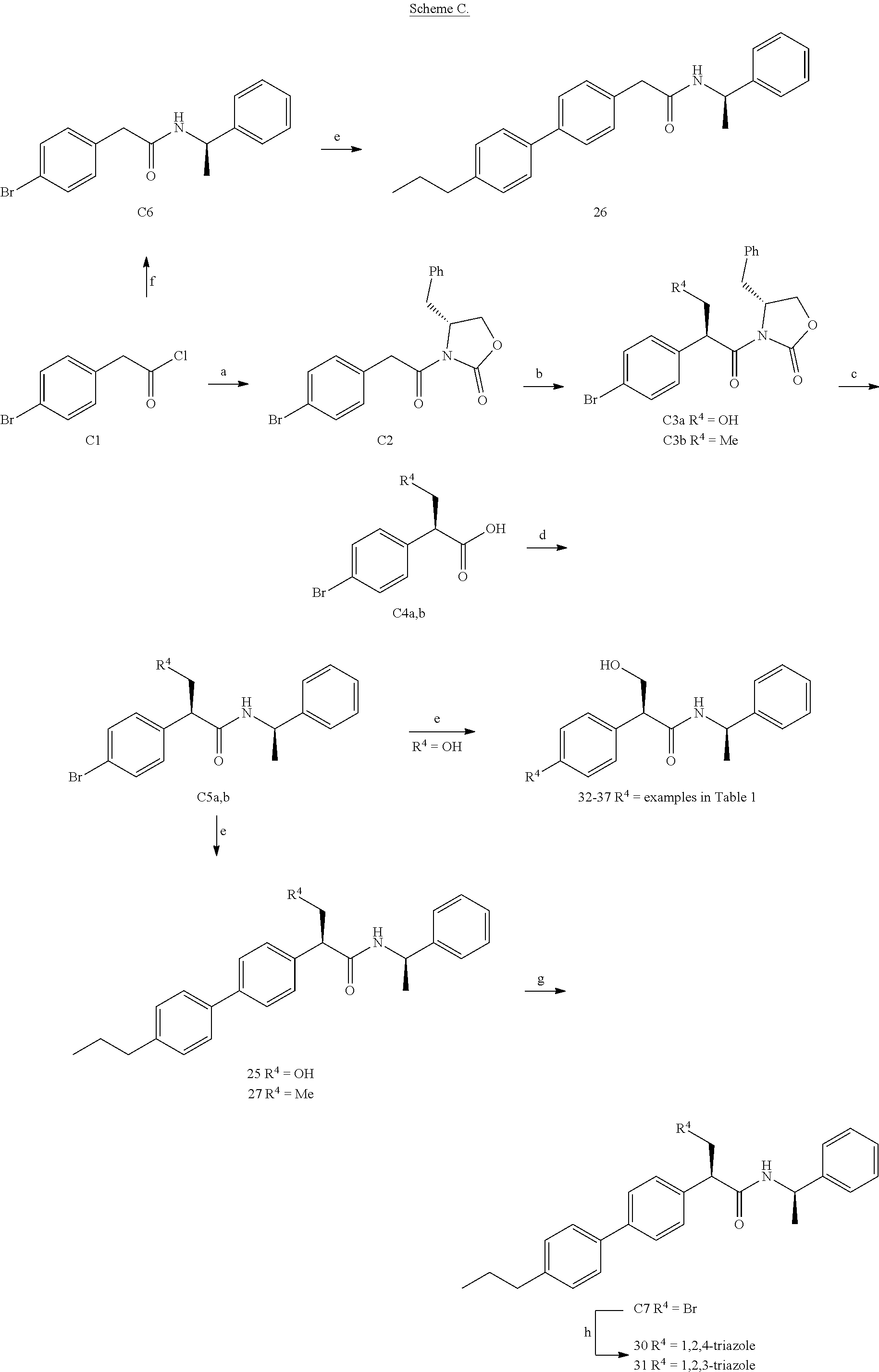

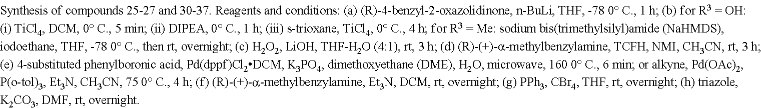

Target compounds 28 and 29 were synthesized in accordance with reactions shown in Scheme D. Reaction of 4-iodobromobenzene D1 with tert-butyl methyl malonate in the presence of NaH and CuBr afforded 4-bromophenyl malonate D2. The tert-butyl group was removed with HCl to give acid D3, which was treated with oxalyl chloride to form the corresponding acid chloride (structure not shown). The acid chloride was coupled with (R)-(+)-α-methylbenzylamine to provide amide D4. Suzuki coupling of D4 with 4-propyl phenylboronic acid gave the target compound 28. The ester group in 28 was converted to the corresponding acid, followed by coupling with methylamine to provide the target compound 29.

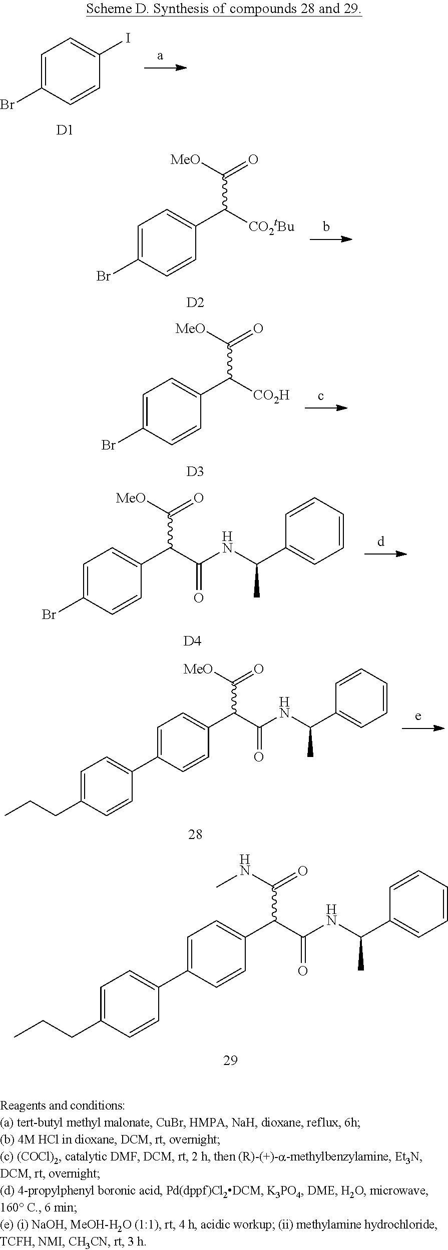

Urea derivatives 38-60 were synthesized in accordance with reactions shown in Scheme E. Substituted aniline E1 and amine (or isocyanate) E2 were coupled by employing standard urea coupling methods. The Boc protected ureas E3a-q were then treated with 4 M HCl in dioxane to remove the Boc protecting group, furnishing the target compounds 38-54. On the other hand, the 4-bromophenyl derivative E3r was subjected to Suzuki coupling with 4-substituted phenyl boronic acids to afford biphenyls E4a-e or Sonogashira coupling with 4-methyl-1-pent-1-yne to afford E4f. Finally, Boc group deprotection with HCl provided the target compounds 55-60.

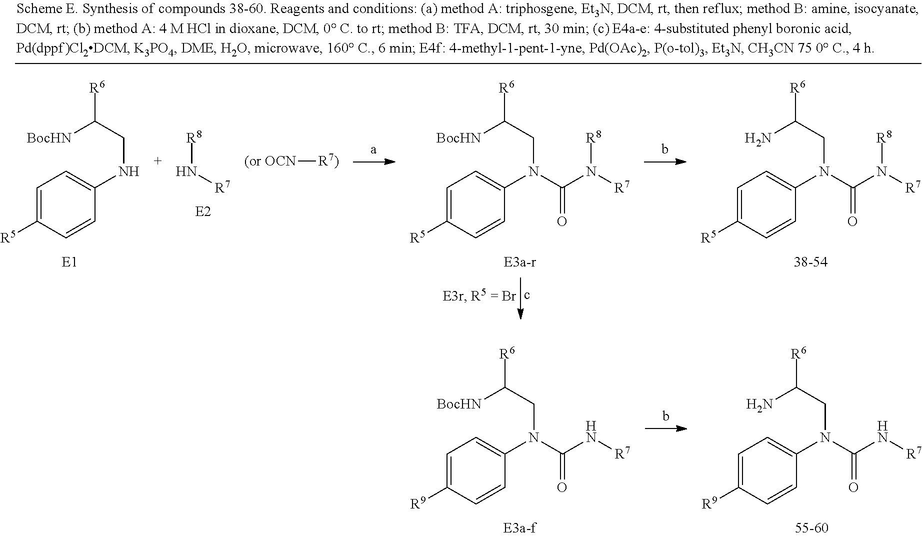

### EXPERIMENTAL

All solvents and chemicals were reagent grade. Unless otherwise mentioned, all reagents and solvents were purchased from commercial vendors and used as received. Flash column chromatography was carried out on a Teledyne ISCO CombiFlash Rf system using prepacked columns. Solvents used include hexane, ethyl acetate (EtOAc), dichloromethane (DCM) and methanol. Purity and characterization of compounds were established by a combination of NMR, mass spectrometry, TLC, and HPLC analyses. ¹H and ¹³C NMR spectra were recorded on a Bruker Avance DPX-300 (300 MHz) or a JEOL 400 MHz NMR spectrometer and were determined in CDCl₃, DMSO-d₆, or CD₃OD with tetramethylsilane (TMS) (0.00 ppm) or solvent peaks as the internal reference. Chemical shifts are reported in ppm relative to the reference signal and coupling constant (J) values are reported in hertz (Hz). Nominal mass spectra were obtained using an Agilent InfinityLab MSD single quadrupole mass spectrometer system (ESI). Thin layer chromatography (TLC) was performed on EMD precoated silica gel 60 F254 plates, and spots were visualized with UV light or iodine staining. All final compounds were greater than 95% pure as determined by HPLC on a Waters 2695 Separation Module equipped with a Waters 2996 Photodiode Array Detector and a Phenomenex Synergi 4 mm Hydro-RP 80A C18 250×4.6 mm column using a flow rate of 1 mL/min starting with 1 min at 5% solvent B, followed by a 15 min gradient of 5-95% solvent B, followed by 9 min at 95% solvent B (solvent A, water with 0.1% TFA; solvent B, acetonitrile with 0.1% TFA and 5% water; absorbance monitored at 280 nm).

### Synthesis of Compounds 3-6 (Scheme A)

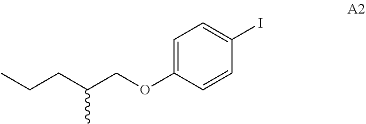

1-Iodo-4-[(2-methylpentyl)oxy]benzene (A2). To a solution of 4-iodophenol (A1) (3.0 g, 13.63 mmol), PPh₃ (6.08 g, 23.18 mmol), and 2-methylpentanol (3.38 mL, 27.27 mmol) in THE (50 mL) at room temperature under nitrogen was slowly added DEAD (3.93 mL, 23.18 mmol) dropwise, while keeping the reaction temperature below 35° C. After addition, the reaction mixture was stirred at room temperature overnight and quenched with H₂O (10 mL). The layers were separated, and the aqueous layer was extracted with EtOAc (3×30 mL). The combined organic layers were washed with brine (3×30 mL), dried (Na₂SO₄), and concentrated under reduced pressure. The residue was subjected to chromatography on silica gel using 0-10% EtOAc in hexanes to afford A2 (3.56 g, 86% yield) as a light-yellow oil: ¹H NMR (300 MHz, CDCl₃) δ 7.56-7.31 (m, 2H), 6.68-6.41 (m, 2H), 3.64 (ddd, J=15.6, 8.9, 6.3 Hz, 2H), 2.03-1.73 (m, 1H), 1.55-1.13 (m, 4H), 0.97 (d, J=6.7 Hz, 3H), 0.90 (t, J=7.1 Hz, 3H); ¹³C NMR (75 MHz, CDCl₃) δ 159.3, 138.2, 117.1, 82.6, 73.5, 35.9, 33.0, 20.2, 17.2, 14.6; MS (ESI) m/z 305.2 [M+H]⁺.

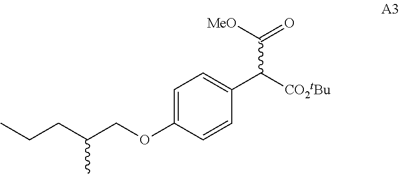 [SMILES: CCCC(C)COc1ccc(C(C(=O)OC)C(=O)OC(C)(C)C)cc1]

1-tert-Butyl 3-Methyl-2-{4-[(2-methylpentyl)oxy]phenyl}propanedioate (A3). To a suspension of sodium hydride (60%, 208 mg, 6.25 mmol) in anhydrous dioxane (30 mL) were added HMPA (1.2 mL) and tert-butyl methyl malonate (907 mg, 5.20 mmol). The mixture was stirred at room temperature for 1 h. To the resulting mixture was added copper(I) bromide (896 mg, 6.25 mmol) and A2 (1.9 g, 6.25 mmol), then the mixture was refluxed under nitrogen for 3 h. After cooling to room temperature, the reaction was quenched with saturated NH₄Cl solution (20 mL) and extracted with EtOAc (3×50 mL). The combined organic layers were washed brine (2×50 mL), dried (Na₂SO₄), and concentrated under reduced pressure. The residue was subjected to chromatography on silica gel using 0-20% EtOAc in hexanes to afforded A3 (1.80 g, 99% yield) as a colorless oil: ¹H NMR (300 MHz, CDCl₃) δ 7.28 (d, J=8.7 Hz, 2H), 6.87 (d, J=8.7 Hz, 2H), 4.48 (s, 1H), 3.85-3.66 (m, 5H), 2.02-1.85 (m, 1H), 1.50-1.12 (m, 13H), 0.99 (t, J=5.3 Hz, 3H), 0.96-0.85 (m, 3H); ¹³C NMR (75 MHz, CDCl₃) δ 169.3, 167.5, 159.2, 130.3, 125.0, 114.6, 82.2, 73.2, 58.1, 52.5, 35.8, 32.9, 27.9, 20.0, 17.0, 14.3; MS (ESI) m/z 373.2 [M+Na]⁺.

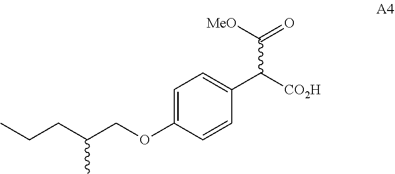 [SMILES: CCCC(C)COc1ccc(C(C(=O)O)C(=O)OC)cc1]

3-Methoxy-2-{4-[(2-methylpentyl)oxy]phenyl}-3-oxopropanoic Acid (A4). To a solution of A3 (550 mg, 1.57 mmol) in DCM (20 mL) was added 4 M HCl in dioxane (3.92 mL, 15.70 mmol), and the resulting reaction was stirred at room temperature overnight. The solvent was removed under reduced pressure to afford A4 (460 mg, quantitative yield) as a colorless oil: ¹H NMR (300 MHz, CDCl₃) δ 7.28 (d, J=6.7 Hz, 2H), 6.88 (d, J=8.7 Hz, 2H), 4.60 (br s, 1H), 3.92-3.60 (m, 5H), 1.93 (dq, J=12.8, 6.5 Hz, 1H), 1.63-1.10 (m, 4H), 1.00 (d, J=6.7 Hz, 3H), 0.92 (t, J=7.0 Hz, 3H); MS (ESI) m/z295.2 [M+H]⁺.

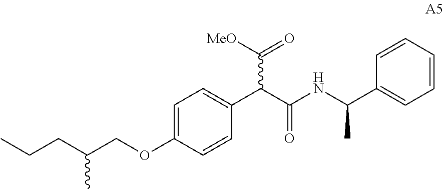 [SMILES: CCCC(C)COc1ccc(C(C(=O)N[C@H](C)c2ccccc2)C(=O)OC)cc1]

Methyl 2-{4-[(2-Methylpentyl)oxy]phenyl}-2-{[(1R)-1-phenylethyl]carbamoyl}acetate (A5). To a solution of A4 (251 mg, 0.85 mmol) in DCM (10 mL) were added oxalyl chloride (0.15 mL, 1.71 mmol) and DMF (1 drop), and the resulting reaction was stirred at room temperature for 4 h. The solvent was evaporated under reduced pressure and the residue was dissolved in DCM (10 mL) followed by the addition of Et₃N (596 μL, 4.28 mmol) and (R)-(+)-α-methylbenzylamine (165 μL, 1.28 mmol). The mixture was stirred at room temperature for 48 h. The reaction was quenched with saturated NaHCO₃ (5 mL). The layers were separated, and the aqueous layer was extracted with additional DCM (2×20 mL). The combined organic layers were washed with brine (2×20 mL), dried (Na₂SO₄), and concentrated under reduced pressure. The residue was subjected to chromatography on silica gel using 0-100% EtOAc in hexanes to afford the diastereomeric mixture of A5 (244 mg, 72% yield) as a colorless oil: ¹H NMR (300 MHz, CDCl₃) δ 7.41-7.07 (m, 8H), 6.93-6.72 (m, 2H), 5.08 (p, J=7.0 Hz, 1H), 4.48 (d, J=1.6 Hz, 1H), 3.87-3.57 (m, 5H), 1.98-1.83 (m, 1H), 1.60-1.10 (m, 7H), 1.00 (dd, J=6.7, 1.8 Hz, 3H), 0.96-0.86 (m, 3H); ¹³C NMR (75 MHz, CDCl₃) δ 166.7, 159.3, 143.0, 129.5, 129.5, 128.6, 128.6, 127.2, 126.0, 114.9, 114.9, 73.2, 57.5, 52.5, 49.0, 48.9, 35.7, 32.9, 21.7, 20.0, 17.0, 14.3; MS (ESI) m/z 398.4 [M+H]⁺.

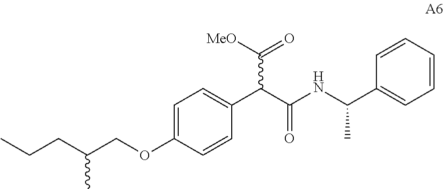 [SMILES: CCCC(C)COc1ccc(C(C(=O)N[C@@H](C)c2ccccc2)C(=O)OC)cc1]

Methyl 2-{4-[(2-Methylpentyl)oxy]phenyl}-2-{[(1S)-1-phenylethyl]carbamoyl}acetate (A6). To a solution A4 (200 mg, 0.68 mmol) in MeCN (5 mL) were added HBTU (387 mg, 1.02 mmol), Et₃N (284 μL, 2.04 mmol), and (S)-(−)-α-methylbenzylamine (131 μL, 1.02 mmol), and the resulting reaction was stirred at room temperature for 6 h. The reaction was quenched with H₂O (10 mL) and extracted with EtOAc (3×10 mL). The combined organic layers were washed with brine (2×10 mL), dried (Na₂SO₄), and concentrated under reduced pressure. The residue was subjected to chromatography on silica gel using 0-100% EtOAc in hexanes to afford diastereomeric mixture A6 (257 mg, 31% yield) as a colorless oil. ¹H NMR (300 MHz, CDCl₃) δ 7.41-7.15 (m, 6H), 7.04 (dd, J=19.6, 7.9 Hz, 1H), 6.86 (t, J=9.2 Hz, 2H), 5.11 (p, J=7.0 Hz, 1H), 4.47 (s, 1H), 3.83-3.66 (m, 5H), 2.01-1.84 (m, 1H), 1.46 (t, J=7.4 Hz, 4H), 1.41-1.12 (m, 3H), 1.07 (br s, 1H), 1.00 (dd, J=6.7, 2.1 Hz, 3H), 0.92 (t, J=6.9 Hz, 3H); ¹³C NMR (75 MHz, CDCl₃) δ 171.5, 166.6, 159.4, 159.4, 143.0, 142.9, 129.4, 129.4, 128.6, 128.6, 127.3, 127.3, 126.0, 125.8, 125.7, 115.1, 115.0, 73.3, 57.7, 57.6, 52.6, 49.0, 49.0, 35.8, 32.9, 21.9, 21.8, 20.0, 17.0, 14.3; MS (ESI) m/z 398.4 [M+H]⁺.

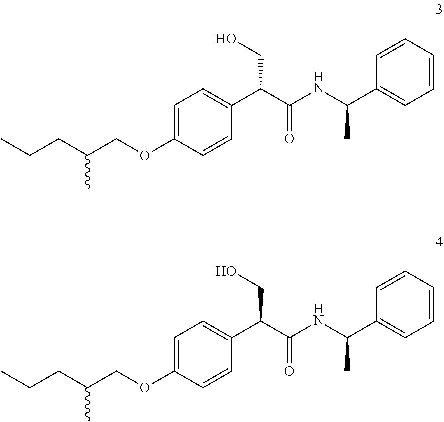

(2R)-3-Hydroxy-2-{4-[(2-methylpentyl)oxy]phenyl}-N-[(1 R)-1-phenylethyl]propanamide (3) and (2S)-3-Hydroxy-2-{4-[(2-methylpentyl)oxy]phenyl}-N-[(1 R)-1-phenylethyl]propenamide (4). To a suspension of NaBH₄ (24 mg, 0.63 mmol) in EtOH (5 mL) at 0° C. under nitrogen was added LiCl (27 mg, 0.63 mmol). After stirring at 0° C. for 10 min, a solution of A5 (100 mg, 0.25 mmol) in THE (5 mL) was added. The reaction mixture was stirred at room temperature for 3 h and quenched with saturated NH₄Cl solution (5 mL), followed by the addition of H₂O (10 mL). The mixture was extracted with EtOAc (3×20 mL). The combined organic layers were washed with brine (20 mL), dried (Na₂SO₄), and concentrated under reduced pressure to afford a diastereomeric mixture of alcohols (41 mg, 44% yield). The mixture of diastereomers was subjected to chromatography on silica gel using 0-100% EtOAc in hexanes to afford pure 3 (assigned as (1 R, 2 R), eluted at 35% EtOAc, 17 mg) as a white solid and pure 4 (assigned as (1 R, 2S), eluted at 45% EtOAc, 24 mg) as a white solid. Compound 3: ¹H NMR (300 MHz, CDCl₃) δ 7.36-7.22 (m, 5H), 7.19-7.10 (m, 2H), 6.96-6.80 (m, 2H), 5.66 (d, J=7.9 Hz, 1H), 5.21-5.01 (m, 1H), 4.20-4.00 (m, 1H), 3.81 (dd, J=8.9, 5.8 Hz, 1H), 3.77-3.66 (m, 2H), 3.59 (dd, J=8.9, 4.4 Hz, 1H), 3.48 (dd, J=8.8, 4.1 Hz, 1H), 2.07-1.80 (m, 1H), 1.55-1.15 (m, 7H), 1.02 (d, J=6.7 Hz, 3H), 0.92 (t, J=7.0 Hz, 3H); ¹³C NMR (75 MHz, CDCl₃) δ 173.2, 159.0, 142.8, 129.5, 128.7, 128.4, 127.4, 126.0, 115.2, 73.3, 65.3, 53.4, 48.8, 35.8, 32.9, 21.5, 20.0, 17.0, 14.3; MS (ESI) m/z370.4 [M+H]⁺; HPLC, >99%, t_{R} 17.3 min. Compound 4: ¹H NMR (300 MHz, CD₃OD) δ 7.23-6.99 (m, 7H), 6.85-6.73 (m, 2H), 5.08-4.92 (m, 1H), 4.19-3.97 (m, 1H), 3.85-3.51 (m, 4H), 2.00-1.74 (m, 1H), 1.55-1.16 (m, 7H), 1.05-0.96 (m, 3H), 0.96-0.84 (m, 3H); ¹³C NMR (75 MHz, CD₃OD) δ 178.1, 164.0, 149.1, 134.6, 134.1, 133.2, 131.7, 130.8, 119.5, 78.2, 68.9, 59.3, 53.9, 40.9, 38.1, 26.5, 25.0, 21.3, 18.6; MS (ESI) m/z 370.4 [M+H]⁺; HPLC, >99%, t_{R} 17.2 min.

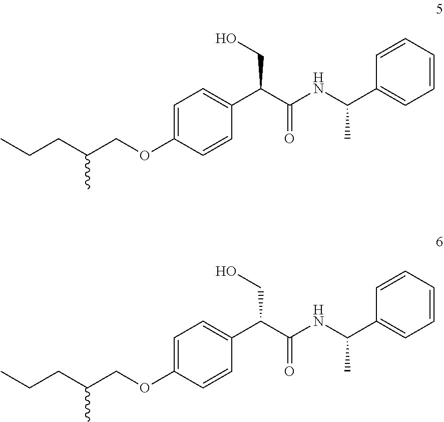

(2S)-3-Hydroxy-2-{4-[(2-methylpentyl)oxy]phenyl}-N-[(1S)-1-phenylethyl]propenamide (5) and (2R)-3-Hydroxy-2-{4-[(2-methylpentyl)oxy]phenyl}-N-[(1 S)-1-phenylethyl]propenamide (6). The procedure for the synthesis of 3 and 4 was followed starting with ester A6 to afford a diastereomeric mixture of alcohols (69% yield). The mixture of diastereomers was subjected to chromatography on silica gel using 0-100% EtOAc in hexanes to afford pure 5 (assigned as (1 S, 2S), 27 mg] as a white solid and pure 6 (assigned as (1 S, 2R), 37 mg) as a white solid. Compound 5: ¹H NMR (300 MHz, CDCl₃) δ 7.36-7.22 (m, 5H), 7.16 (d, J=8.5 Hz, 2H), 6.89 (d, J=8.6 Hz, 2H), 5.65 (d, J=7.6 Hz, 1H), 5.13 (p, J=7.1 Hz, 1H), 4.20-4.01 (m, 1H), 3.81 (dd, J=8.7, 5.9 Hz, 1H), 3.77-3.66 (m, 2H), 3.59 (dd, J=8.8, 4.4 Hz, 1H), 3.46 (dd, J=8.9, 4.2 Hz, 1H), 1.93 (dt, J=12.6, 6.4 Hz, 1H), 1.52-1.28 (m, 6H), 1.27-1.16 (m, 1H), 1.02 (d, J=6.7 Hz, 3H), 0.92 (t, J=7.0 Hz, 3H); ¹³C NMR (75 MHz, CDCl₃) δ 173.2, 159.0, 142.8, 129.5, 128.7, 128.4, 127.4, 126.0, 115.2, 73.3, 65.3, 53.5, 48.8, 35.8, 32.9, 21.5, 20.1, 17.0, 14.3; MS (ESI) m/z370.4 [M+H]⁺; HPLC, >99%, t_{R} 17.3 min. Compound 6: ¹H NMR (300 MHz, CDCl₃) δ 7.32-7.21 (m, 3H), 7.18-7.02 (m, 4H), 6.89-6.79 (m, 2H), 5.68 (d, J=7.6 Hz, 1H), 5.10 (p, J=7.0 Hz, 1H), 4.07 (ddd, J=10.7, 8.8, 4.1 Hz, 1H), 3.80 (dd, J=8.9, 5.8 Hz, 1H), 3.76-3.58 (m, 3H), 3.51 (dd, J=8.9, 4.1 Hz, 1H), 2.05-1.80 (m, 1H), 1.54-1.16 (m, 7H), 1.01 (d, J=6.7 Hz, 3H), 0.92 (t, J=7.0 Hz, 3H); ¹³C NMR (75 MHz, CDCl₃) δ 173.2, 159.1, 143.1, 129.6, 128.6, 128.3, 127.3, 125.8, 115.2, 73.4, 65.2, 53.4, 48.9, 35.8, 32.9, 22.0, 20.1, 17.0, 14.3; MS (ESI) m/z 370.4 [M+H]⁺; HPLC, >99%, t_{R} 17.2 min.

### Synthesis of Compounds 4, 5, and 7-24 (Scheme B)

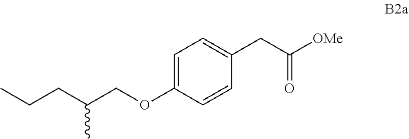 [SMILES: CCCC(C)COc1ccc(CC(=O)OC)cc1]

Methyl 2-{4-[(2-methylpentyl)oxy]phenyl}acetate (B2a). The procedure for the synthesis of A2 was followed starting with B1 to afford ether B2a (70% yield) as a colorless oil. ¹H NMR (300 MHz, CDCl₃) δ 7.20-7.10 (m, 2H), 6.92-6.72 (m, 2H), 3.85-3.62 (m, 5H), 3.55 (s, 2H), 2.04-1.79 (m, 1H), 1.59-1.08 (m, 4H), 1.00 (d, J=6.7 Hz, 3H), 0.91 (t, J=7.1 Hz, 3H); ¹³C NMR (75 MHz, CDCl₃) δ 172.4, 158.5, 130.2, 125.8, 114.6, 73.3, 51.9, 40.3, 35.8, 32.9, 20.0, 17.0, 14.3; MS (ESI) m/z 251.2 [M+H]⁺.

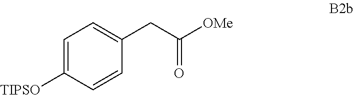

Methyl 2-(4-{[Tris(propan-2-yl)silyl]oxy}phenyl)acetate (B2b). To a solution of B1 (5.0 g, 30.08 mmol) in DMF (50 mL) were added TIPS chloride (7.09 mL, 33.09 mmol) and imidazole (4.50 g, 66.18 mmol), and the resulting reaction was stirred at room temperature overnight. The mixture was diluted with H₂O (100 mL) and extracted with DCM (3×50 mL). The combined organic layers were washed with brine (2×50 mL), dried (Na₂SO₄), and concentrated under reduced pressure. The residue was subjected to chromatography on silica gel using 0-10% EtOAc in hexanes to afford B2b (9.4 g, 97% yield) as a colorless oil: ¹H NMR (300 MHz, CDCl₃) δ 7.11 (d, J=8.5 Hz, 2H), 6.82 (d, J=8.5 Hz, 2H), 3.67 (s, 3H), 3.54 (s, 2H), 1.35-1.17 (m, 3H), 1.17-0.97 (m, 18H); MS ESI) m/z323.2 [M+H]⁺.

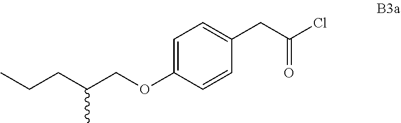 [SMILES: CCCC(C)COc1ccc(CC(=O)Cl)cc1]

2-{4-[(2-Methylpentyl)oxy]phenyl}acetyl Chloride (B3a). To a solution of B2a (5.0 g, 20.0 mmol) in EtOH—H₂O (60 mL, 2:1) was added 1 N NaOH (40 mL, 40.0 mmol), and the reaction was stirred at room temperature for 3 h. The mixture was acidified to pH=2 with 1 N HCl and extracted with EtOAc (3×10 mL). The combined organic layers were washed with brine (3×10 mL), dried (Na₂SO₄), and concentrated under reduced pressure to give the corresponding acid (5 g, quantitative). To a solution of the acid (5.0 g, 21.1 mmol) in DCM (50 mL) were added oxalyl chloride (3.91 mL, 42.32 mmol) and DMF (0.2 mL), and the resulting reaction was stirred at room temperature for 4 h. The solvent was removed under reduced pressure and the residue was dried in vacuo to afford crude B3a as a yellow residue, which was used immediately for the next transformation without characterization.

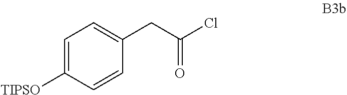

2-(4-{[Tris(propan-2-yl)silyl]oxy}phenyl)acetyl Chloride (B3b). The procedure for the synthesis of B3a was followed starting with B2b to afford B3b (99% yield) as a yellow residue, which was used immediately for the next transformation without characterization.

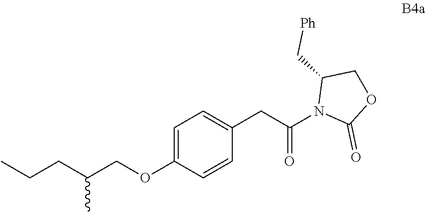 [SMILES: CCCC(C)COc1ccc(CC(=O)N2C(=O)OC[C@H]2Cc2ccccc2)cc1]

(4R)-4-Benzyl-3-(2-{4-[(2-methylpentyl)oxy]phenyl}acetyl)-1,3-oxazolidin-2-one (B4a). To a solution of (R)-4-benzyl-2-oxazolidinone (3.56 g, 20.1 mmol) in THE (40 mL) at −78° C. was slowly added n-BuLi (2.5 M in hexanes, 8.46 mL, 21.10 mmol) dropwise over 10 min. After stirring for additional 10 min at −78° C., a solution of B3a (5.0 g) in 10 mL THF) was added to the above reaction over 10 min at −78° C. The resulting reaction was stirred at −78° C. for 15 min and allowed to warm to room temperature and stirred an additional 1 h. The reaction was quenched with saturated aqueous NaHCO₃ (50 mL) and layers were separated. The aqueous layer was extracted with EtOAc (2×50 mL). The combined organic layers were washed with brine (2×50 mL), dried (Na₂SO₄), and concentrated under reduced pressure. The residue was subjected to chromatography on silica gel using 0-100% EtOAc in hexanes to afforded B4a (6.43 g, 77% yield) as white solid: ¹H NMR (300 MHz, CDCl₃) δ 7.34-7.16 (m, 5H), 7.15-7.03 (m, 2H), 6.96-6.77 (m, 2H), 4.77-4.49 (m, 1H), 4.30-4.10 (m, 4H), 3.88-3.58 (m, 2H), 3.23 (dd, J=13.4, 3.1 Hz, 1H), 2.74 (dd, J=13.3, 9.4 Hz, 1H), 2.01-1.83 (m, 1H), 1.58-1.12 (m, 4H), 1.00 (d, J=6.7 Hz, 3H), 0.91 (t, J=7.0 Hz, 3H); ¹³C NMR (75 MHz, CDCl₃) δ 171.6, 158.6, 153.4, 135.2, 130.8, 129.4, 128.9, 127.3, 125.3, 114.7, 73.2, 66.1, 55.3, 40.7, 37.7, 35.8, 32.9, 20.1, 17.0, 14.3; MS (ESI) m/z396.4 [M+H]⁺.

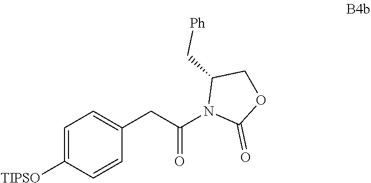

(4R)-4-Benzyl-3-[2-(4-{[tris(propan-2-yl)silyl]oxy}phenyl)acetyl]-1,3-oxazolidin-2-one (B4b). The procedure for the synthesis of B4a was followed starting with B3b to afford B4b (70% yield) as a colorless oil: ¹H NMR (300 MHz, CDCl₃) δ 7.38-6.97 (m, 7H), 6.77 (d, J=8.5 Hz, 2H), 4.69-4.48 (m, 1H), 4.28-3.95 (m, 4H), 3.15 (dd, J=13.4, 3.2 Hz, 1H), 2.65 (dd, J=13.4, 9.4 Hz, 1H), 1.31-1.07 (m, 3H), 1.07-0.93 (m, 18H); MS (ESI) m/z 468.6 [M+H]⁺.

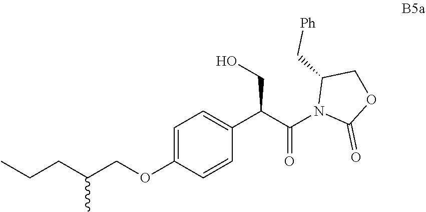 [SMILES: CCCC(C)COc1ccc([C@@H](CO)C(=O)N2C(=O)OC[C@H]2Cc2ccccc2)cc1]

(4R)-4-Benzyl-3-[(2S)-3-hydroxy-2-{4-[(2-methylpentyl)oxy]phenyl}propanoyl]-1,3-oxazolidin-2-one (B5a). To a solution of B4a (6.38 g, 16.13 mmol) in DCM (120 mL) at 0° C. was slowly added TiCl₄ (1 M in toluene, 16.94 mL, 16.94 mmol) dropwise over 5 min. The resulting brown solution was stirred for 5 min at 0° C., after which DIPEA (3.09 mL, 417.74 mmol) was added dropwise. The resulting deep-blue mixture was stirred 0° C. for 1 h. Afterwards, a solution of s-trioxane (1.67 g, 18.55 mmol) in DCM (5 mL) and additional TiCl₄ (16.94 mL, 16.94 mmol) were added successively. The mixture was stirred 0° C. for an additional 4 h and then quenched with saturated aqueous NH₄Cl and extracted with additional DCM (3×20 mL). The combined organic layers were washed with brine (2×50 mL), dried (Na₂SO₄), and concentrated under reduced pressure. The residue was subjected to chromatography on silica gel using 0-100% EtOAc in hexanes to afford B5a (2.2 g, 32% yield) as a colorless oil: ¹H NMR (300 MHz, CDCl₃) δ 7.33-6.99 (m, 7H), 6.74 (d, J=8.5 Hz, 2H), 5.11 (dd, J=9.2, 5.1 Hz, 1H), 4.63-4.39 (m, 1H), 4.25-4.04 (m, 1H), 4.04-3.83 (m, 2H), 3.79-3.51 (m, 3H), 3.22 (dd, J=13.4, 3.2 Hz, 1H), 2.76 (dd, J=13.4, 9.3 Hz, 1H), 2.42 (br s, 1H), 1.90-1.69 (m, 1H), 1.42-1.08 (m, 4H), 0.90 (d, J=6.7 Hz, 3H), 0.87-0.78 (m, 3H); ¹³C NMR (75 MHz, CDCl₃) δ 173.4, 159.0, 152.7, 135.3, 129.9, 129.5, 129.0, 127.3, 126.6, 114.7, 73.2, 65.9, 65.2, 55.6, 51.1, 37.9, 35.7, 32.9, 20.0, 17.0, 14.3; MS (ESI) m/z 408.4 [M−H₂O+H]⁺.

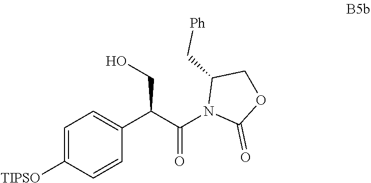

(4R)-4-Benzyl-3-[(2S)-3-hydroxy-2-(4-{[tris(propan-2-yl)silyl]oxy}phenyl)propanoyl]-1,3-oxazolidin-2-one (B5b). The procedure for the synthesis of B5a was followed starting with B4b to afford B5b (59% yield) as a white foam: ¹H NMR (300 MHz, CDCl₃) δ ¹H NMR (300 MHz, CDCl₃) δ 7.38-7.10 (m, 7H), 6.86-6.76 (m, 2H), 5.18 (dd, J=9.2, 5.1 Hz, 1H), 4.72-4.53 (m, 1H), 4.28-4.11 (m, 2H), 3.91-3.70 (m, 1H), 3.33 (dd, J=13.4, 3.4 Hz, 1H), 2.86 (dd, J=13.4, 9.4 Hz, 1H), 2.11 (t, J=6.5 Hz, 1H), 1.34-1.16 (m, 4H), 1.14-1.00 (m, 18H); MS (ESI) m/z 480.0 [M−H₂O+H]⁺.

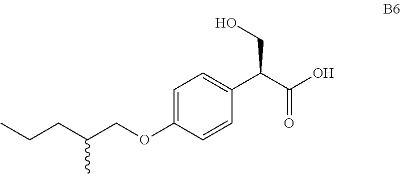 [SMILES: CCCC(C)COc1ccc([C@@H](CO)C(=O)O)cc1]

(2S)-3-Hydroxy-2-{4-[(2-methylpentyl)oxy]phenyl}propanoic acid (B6). To a solution of B5a (2.21 g, 5.2 mmol) in THF—H₂O (4:1, 50 mL) at 0° C. were added LiOH·H₂O (436 mg, 10.40 mmol) and H₂O₂ (30%, 5.89 mL, 52.0 mmol), and the resulting reaction was stirred at 0° C. for 4 h. The mixture was partitioned between DCM and H₂O. The aqueous layer was acidified with 1 N HCl to pH=2 and extracted with EtOAc (3×50 mL). The combined organic layers were washed with brine (3×50 mL), dried (Na₂SO₄), and concentrated under reduced pressure to afford crude B6 (quantitative yield) as a white solid, which was used for the next transformation without further purification. ¹H NMR (300 MHz, CDCl₃) δ 7.40-6.98 (m, 3H), 6.84 (d, J=8.5 Hz, 2H), 4.24-3.97 (m, 1H), 3.89-3.57 (m, 4H), 2.00-1.81 (m, 1H), 1.58-1.08 (m, 4H), 0.99 (d, J=6.7 Hz, 3H), 0.91 (t, J=7.0 Hz, 3H); MS (ESI) m/z 265.2 [M−H]⁻.

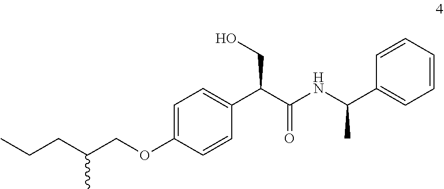 [SMILES: CCCC(C)COc1ccc([C@@H](CO)C(=O)N[C@H](C)c2ccccc2)cc1]

(2S)-3-Hydroxy-2-{4-[(2-methylpentyl)oxy]phenyl}-N-[(1 R)-1-phenylethyl]propenamide (4). Method A: To a solution of B6 (95 mg, 0.36 mmol) in MeCN (5 mL) at room temperature were added DIPEA (0.19 mL, 1.08 mmol), (R)-(+)-α-methylbenzylamine (65 mg, 0.54 mmol), and HBTU (203 mg, 0.54 mmol). After stirring for 5 h, the reaction was quenched with H₂O (10 mL), followed by addition of EtOAc (50 mL). The layers were separated. The organic layer was washed with saturated NaHCO₃ (10 mL) and brine (2×20 mL), dried (Na₂SO₄), and concentrated under reduced pressure. The residue was subjected to chromatography on silica gel using 0-100% EtOAc in hexanes to afford 4 (90 mg, 68% yield) as a white solid. Method B: To a solution of B6 (30 mg, 0.11 mmol) in MeCN (1 mL) at room temperature were added (R)-(+)-α-methylbenzylamine (20.5 mg, 0.17 mmol), TCFH (35 mg, 0.12 mmol), and NMI (20.4 mg, 0.25 mmol), and the reaction was stirred at room temperature for 3 h. The solvent was removed under reduced pressure. The residue was subjected to chromatography on silica gel using 0-100% EtOAc in hexanes to afford 4 (30 mg, 72% yield) as a white solid. This material has the identical ¹H NMR and ¹³C NMR to those of 4 prepared in Scheme 1.

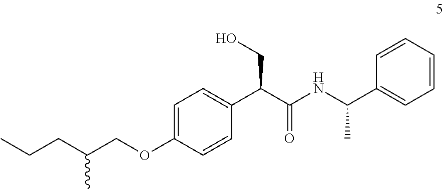 [SMILES: CCCC(C)COc1ccc([C@@H](CO)C(=O)N[C@@H](C)c2ccccc2)cc1]

(2S)-3-Hydroxy-2-{4-[(2-methylpentyl)oxy]phenyl}-N-[(1 S)-1-phenylethyl]propenamide (5). The procedure for the synthesis of 4 was followed starting with B6 and (S)-(−)-α-methylbenzylamine to afford 5 (70% yield) as a white solid. This material has the identical ¹H NMR and ¹³C NMR to those of 5 prepared in Scheme 1.

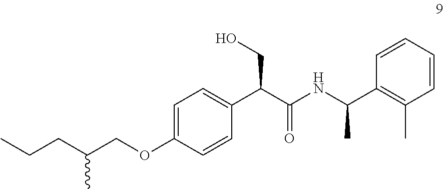 [SMILES: CCCC(C)COc1ccc([C@@H](CO)C(=O)N[C@H](C)c2ccccc2C)cc1]

(2S)-3-Hydroxy-2-{4-[(2-methylpentyl)oxy]phenyl}-N-[(1 R)-1-(2-methylphenyl)ethyl]propenamide (9). The procedure for the synthesis of 4 was followed starting with B6 and (1 R)-1-(2-methylphenyl)ethan-1-amine to afford 9 (75% yield) as a white solid: ¹H NMR (300 MHz, CDCl₃) δ 7.17-7.07 (m, 5H), 7.03-6.95 (m, 1H), 6.94-6.74 (m, 2H), 5.75 (d, J=7.4 Hz, 1H), 5.39-5.14 (m, 1H), 4.07 (t, J=10.0 Hz, 1H), 3.82 (dd, J=8.9, 5.8 Hz, 1H), 3.77-3.66 (m, 2H), 3.63 (dd, J=8.7, 4.4 Hz, 1H), 3.56-3.43 (m, 1H), 2.33 (s, 3H), 2.07-1.86 (m, 1H), 1.60-1.11 (m, 7H), 1.04 (d, J=6.7 Hz, 3H), 0.95 (t, J=7.0 Hz, 3H); ¹³C NMR (75 MHz, CDCl₃) δ 172.9, 159.0, 141.0, 135.4, 130.7, 129.5, 128.3, 127.2, 126.2, 124.4, 115.1, 73.3, 65.2, 53.4, 46.7, 35.8, 32.9, 21.2, 20.0, 18.9, 17.0, 14.3; MS (ESI) m/z 384.4 [M+H]⁺; HPLC, >97%, t_{R} 18.0 min.

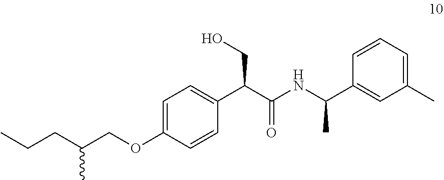 [SMILES: CCCC(C)COc1ccc([C@@H](CO)C(=O)N[C@H](C)c2cccc(C)c2)cc1]

(2S)-3-Hydroxy-2-{4-[(2-methylpentyl)oxy]phenyl}-N-[(1 R)-1-(3-methylphenyl)ethyl]propenamide (10). The procedure for the synthesis of 4 was followed starting with B6 and (1 R)-1-(3-methylphenyl)ethan-1-amine to afford 10 (70% yield) as a white solid: ¹H NMR (300 MHz, CDCl₃) δ 7.24-7.09 (m, 3H), 7.05 (d, J=7.6 Hz, 1H), 6.97-6.81 (m, 4H), 5.74 (d, J=7.7 Hz, 1H), 5.08 (p, J=7.0 Hz, 1H), 4.14-4.02 (m, 1H), 3.89-3.48 (m, 5H), 2.30 (s, 3H), 2.06-1.83 (m, 1H), 1.63-1.11 (m, 7H), 1.03 (d, J=6.7 Hz, 3H), 0.94 (t, J=7.0 Hz, 3H); ¹³C NMR (75 MHz, CDCl₃) δ 173.3, 159.0, 142.9, 138.2, 129.5, 128.9, 128.5, 128.0, 126.4, 122.7, 115.1, 73.3, 65.0, 53.2, 48.9, 35.7, 32.9, 22.1, 21.4, 20.0, 17.0, 14.3; MS (ESI) m/z 384.6 [M+H]⁺; HPLC, >98%, t_{R} 18.0 min.

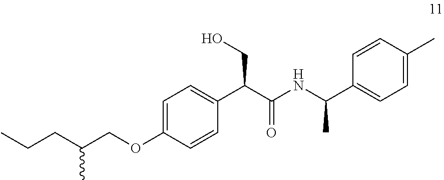 [SMILES: CCCC(C)COc1ccc([C@@H](CO)C(=O)N[C@H](C)c2ccc(C)cc2)cc1]

(2S)-3-Hydroxy-2-{4-[(2-methylpentyl)oxy]phenyl}-N-[(1 R)-1-(4-methylphenyl)ethyl]propenamide (11). The procedure for the synthesis of 4 was followed starting with B6 and (1 R)-1-(4-methylphenyl) ethan-1-amine to afford 11 (80% yield) as a white solid: ¹H NMR (300 MHz, CDCl₃) δ 7.22-6.97 (m, 6H), 6.88 (d, J=8.7 Hz, 2H), 5.72 (d, J=7.7 Hz, 1H), 5.08 (p, J=7.0 Hz, 1H), 4.08 (t, J=9.7 Hz, 1H), 3.91-3.35 (m, 5H), 2.33 (s, 3H), 2.02-1.85 (m, 1H), 1.59-1.12 (m, 7H), 1.08-0.98 (m, 3H), 0.95 (t, J=7.0 Hz, 3H); ¹³C NMR (75 MHz, CDCl₃) δ 173.2, 159.0, 140.1, 136.9, 129.6, 129.2, 128.3, 125.7, 115.1, 73.2, 65.2, 53.4, 48.6, 35.8, 32.9, 22.0, 21.0, 20.0, 17.0, 14.3; MS (ESI) m/z384.4 [M+H]⁺; HPLC, >97%, t_{R} 18.0 min.

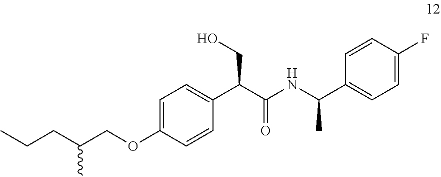 [SMILES: CCCC(C)COc1ccc([C@@H](CO)C(=O)N[C@H](C)c2ccc(F)cc2)cc1]

(2S)—N-[(1 R)-1-(4-Fluorophenyl)ethyl]-3-hydroxy-2-{4-[(2-methylpentyl)oxy]phenyl}propenamide (12). The procedure for the synthesis of 4 was followed starting with B6 and (1 R)-1-(4-fluorophenyl) ethan-1-amine to afford 12 (76% yield) as a white solid: ¹H NMR (300 MHz, CDCl₃) δ 7.14-7.06 (m, 4H), 7.02-6.91 (m, 2H), 6.90-6.79 (m, 2H), 5.77 (d, J=7.6 Hz, 1H), 5.08 (p, J=7.1 Hz, 1H), 4.07 (dd, J=10.9, 8.8 Hz, 1H), 3.86-3.56 (m, 5H), 2.05-1.83 (m, 1H), 1.61-1.16 (m, 7H), 1.03 (d, J=6.7 Hz, 3H), 0.94 (t, J=7.0 Hz, 3H); ¹³C NMR (75 MHz, CDCl₃) δ 173.1, 161.9 (d, J_{C,F}=245.0 Hz), 159.0, 138.9 (d, J_{C,F}=3.0 Hz), 129.5, 128.2, 127.4 (d, J_{C,F}=8.0 Hz), 115.3 (d, J_{C,F}=21.0 Hz), 115.1, 73.3, 65.0, 53.4, 48.2, 35.7, 32.9, 22.0, 20.0, 17.0, 14.3; MS (ESI) m/z 388.4 [M+H]⁺; HPLC, >98%, t_{R} 17.4 min.

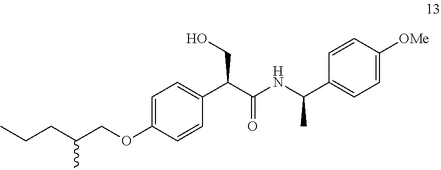 [SMILES: CCCC(C)COc1ccc([C@@H](CO)C(=O)N[C@H](C)c2ccc(OC)cc2)cc1]

(2S)-3-Hydroxy-N-[(1 R)-1-(4-methoxyphenyl)ethyl]-2-{4-[(2-methylpentyl)oxy]phenyl}propenamide (13). The procedure for the synthesis of 4 was followed starting with B6 and (1 R)-1-(4-methoxyphenyl) ethan-1-amine to afford 13 (72% yield) as a white solid: ¹H NMR (300 MHz, CDCl₃) δ 7.16-6.99 (m, 4H), 6.92-6.73 (m, 4H), 5.67 (d, J=7.7 Hz, 1H), 5.07 (p, J=7.0 Hz, 1H), 4.15-4.00 (m, 1H), 3.85-3.76 (m, 4H), 3.75-3.48 (m, 4H), 2.05-1.85 (m, 1H), 1.58-1.12 (m, 7H), 1.03 (d, J=6.7 Hz, 3H), 0.94 (t, J=7.0 Hz, 3H); ¹³C NMR (75 MHz, CDCl₃) δ 173.1, 159.0, 158.7, 135.1, 129.5, 128.3, 127.0, 115.1, 113.9, 73.3, 65.2, 55.2, 53.4, 48.3, 35.7, 32.9, 21.9, 20.0, 17.0, 14.3; MS (ESI) m/z400.4 [M+H]⁺; HPLC, >95%, t_{R} 17.0 min.

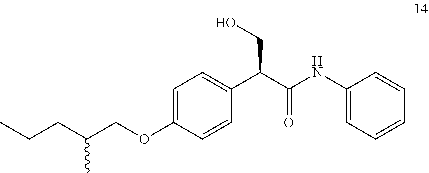 [SMILES: CCCC(C)COc1ccc([C@@H](CO)C(=O)Nc2ccccc2)cc1]

(2S)-3-Hydroxy-2-{4-[(2-methylpentyl)oxy]phenyl}-N-phenylpropanamide (14). The procedure for the synthesis of 4 was followed starting with B6 and aniline to afford 14 (75% yield) as a white solid: ¹H NMR (300 MHz, CDCl₃) δ 7.34 (d, J=8.1 Hz, 2H), 7.26-7.10 (m, 4H), 7.02 (t, J=7.4 Hz, 1H), 6.83 (d, J=8.6 Hz, 2H), 4.21-4.04 (m, 1H), 3.81-3.56 (m, 4H), 3.33-3.09 (m, 1H), 1.97-1.78 (m, 1H), 1.51-1.04 (m, 5H), 0.94 (d, J=6.7 Hz, 3H), 0.85 (t, J=7.0 Hz, 3H); ¹³C NMR (75 MHz, CDCl₃) δ 172.2, 159.2, 140.4, 129.6, 129.0, 127.9, 124.6, 120.0, 115.3, 73.3, 65.0, 54.5, 35.7, 32.9, 20.0, 17.0, 14.3; MS (ESI) m/z342.4 [M+H]⁺; HPLC, >97%, t_{R} 17.3 min.

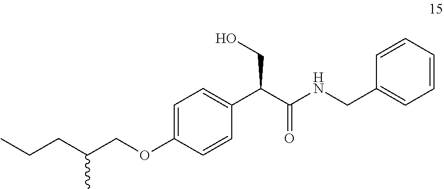 [SMILES: CCCC(C)COc1ccc([C@@H](CO)C(=O)NCc2ccccc2)cc1]

(2S)—N-Benzyl-3-hydroxy-2-{4-[(2-methylpentyl)oxy]phenyl}propenamide (15). The procedure for the synthesis of 4 was followed starting with B6 and benzylamine to afford 15 (65% yield) as a white solid: ¹H NMR (300 MHz, CDCl₃) δ 7.27-7.13 (m, 3H), 7.13-7.03 (m, 4H), 6.83-6.71 (m, 2H), 5.79 (s, 1H), 4.45-4.21 (m, 2H), 4.15-4.00 (m, 1H), 3.79-3.49 (m, 4H), 1.96-1.76 (m, 1H), 1.48-1.02 (m, 5H), 0.93 (d, J=6.7 Hz, 3H), 0.84 (t, J=7.0 Hz, 3H); ¹³C NMR (75 MHz, CDCl₃) δ 174.0, 159.0, 137.9, 129.5, 128.7, 128.3, 127.5, 115.1, 73.3, 65.1, 53.5, 43.4, 35.7, 32.9, 20.0, 17.0, 14.3; MS (ESI) m/z356.4 [M+H]⁺; HPLC, >95%, t_{R} 16.9 min.

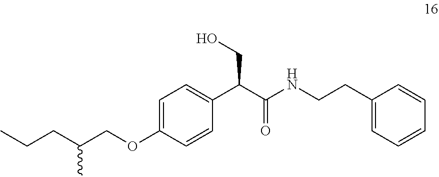 [SMILES: CCCC(C)COc1ccc([C@@H](CO)C(=O)NCCc2ccccc2)cc1]

(2S)-3-Hydroxy-2-{4-[(2-methylpentyl)oxy]phenyl}-N-(2-phenylethyl)propenamide (16). The procedure for the synthesis of 4 was followed starting with B6 and 2-phenylethan-1-amine to afford 16 (60% yield) as a white solid: ¹H NMR (300 MHz, CDCl₃) δ 7.23-7.05 (m, 3H), 7.04-6.89 (m, 4H), 6.76 (d, J=8.7 Hz, 2H), 5.60-5.44 (m, 1H), 4.11-3.93 (m, 1H), 3.80-3.66 (m, 2H), 3.53-3.38 (m, 2H), 3.38-3.20 (m, 1H), 2.70-2.59 (m, 2H), 2.02-1.79 (m, 2H), 1.52-1.18 (m, 5H), 1.01-0.90 (m, 3H), 0.85 (t, J=7.0 Hz, 3H); ¹³C NMR (75 MHz, CDCl₃) δ 174.0, 159.0, 138.5, 129.4, 128., 128.5, 128.3, 126.4, 115.1, 73.3, 64.9, 53.4, 40.5, 35.7, 35.4, 32.9, 20.0, 17.0, 14.3; MS (ESI) m/z 370.4 [M+H]⁺; HPLC, >99%, t_{R} 17.4 min.

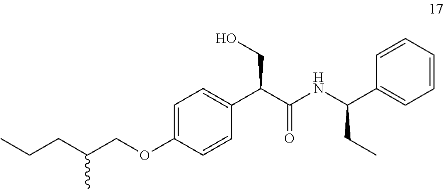 [SMILES: CCCC(C)COc1ccc([C@@H](CO)C(=O)N[C@H](CC)c2ccccc2)cc1]

(2S)-3-Hydroxy-2-{4-[(2-methylpentyl)oxy]phenyl}-N-[(1 R)-1-phenylpropyl]propenamide (17). The procedure for the synthesis of 4 was followed starting with B6 and (1 R)-1-phenylpropan-1-amine to afford 17 (68% yield) as a white solid: ¹H NMR (300 MHz, CDCl₃) δ 7.42-7.16 (m, 4H), 7.10 (d, J=8.6 Hz, 3H), 6.91-6.78 (m, 2H), 5.76 (d, J=8.1 Hz, 1H), 4.87 (q, J=7.5 Hz, 1H), 4.12-3.98 (m, 1H), 3.91-3.45 (m, 5H), 2.06-1.83 (m, 1H), 1.78-1.65 (m, 2H), 1.56-1.21 (m, 4H), 1.07-0.99 (m, 3H), 0.95 (t, J=7.0 Hz, 3H), 0.87 (t, J=7.4 Hz, 3H); ¹³C NMR (75 MHz, CDCl₃) δ 173.5, 159.0, 142.0, 129.6, 128.5, 128.3, 127.2, 126.2, 115.1, 73.3, 65.2, 55.0, 53.3, 35.7, 32.9, 29.3, 20.0, 17.0, 14.3, 10.6; MS (ESI) m/z384.4 [M+H]⁺; HPLC, >95%, t_{R} 17.6 min.

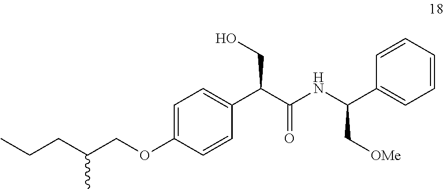 [SMILES: CCCC(C)COc1ccc([C@@H](CO)C(=O)N[C@H](COC)c2ccccc2)cc1]

(2S)-3-Hydroxy-N-[(1 S)-2-methoxy-1-phenylethyl]-2-{4-[(2-methylpentyl)oxy]phenyl}propenamide (18). The procedure for the synthesis of 4 was followed starting with B6 and (1 S)-2-methoxy-1-phenyl ethan-1-amine to afford 18 (50% yield) as a white solid: ¹H NMR (300 MHz, CDCl₃) δ 7.24-7.10 (m, 3H), 7.10-6.97 (m, 4H), 6.80 (d, J=8.7 Hz, 2H), 6.16 (d, J=7.7 Hz, 1H), 5.15-4.99 (m, 1H), 4.05-3.85 (m, 1H), 3.79-3.57 (m, 4H), 3.56-3.39 (m, 2H), 3.21 (s, 3H), 2.00-1.70 (m, 1H), 1.48-1.03 (m, 4H), 0.95 (d, J=6.7 Hz, 3H), 0.85 (t, J=7.0 Hz, 3H); ¹³C NMR (75 MHz, CDCl₃) δ 173.5, 159.0, 139.5, 129.5, 128.5, 128.3, 127.4, 126.4, 115.0, 75.0, 73.3, 65.1, 59.0, 53.4, 52.7, 35.8, 32.9, 20.0, 17.0, 14.3; MS (ESI) m/z400.4 [M+H]⁺; HPLC, >98%, t_{R} 17.1 min.

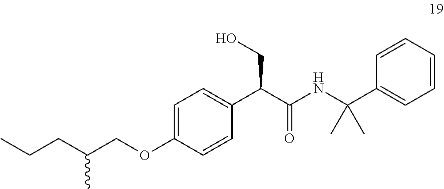 [SMILES: CCCC(C)COc1ccc([C@@H](CO)C(=O)NC(C)(C)c2ccccc2)cc1]

(2S)-3-Hydroxy-2-{4-[(2-methylpentyl)oxy]phenyl}-N-(2-phenylpropan-2-yl)propenamide (19). The procedure for the synthesis of 4 was followed starting with B6 and 2-phenylpropan-2-amine to afford 19 (65% yield) as a white solid: ¹H NMR (300 MHz, CDCl₃) δ 7.42-7.12 (m, 7H), 7.00-6.83 (m, 2H), 5.73 (s, 1H), 4.05 (t, J=9.8 Hz, 1H), 3.90-3.52 (m, 4H), 3.41 (br s, 1H), 2.11-1.82 (m, 1H), 1.65 (s, 3H), 1.61 (s, 3H), 1.55-1.20 (m, 4H), 1.05 (d, J=6.7 Hz, 3H), 0.95 (t, J=7.0 Hz, 3H); ¹³C NMR (75 MHz, CDCl₃) δ 172.9, 159.0, 146.6, 129.4, 128.7, 128.4, 126.7, 124.5, 115.1, 73.3, 65.2, 56.0, 53.9, 35.8, 32.9, 29.3, 28.8, 20.0, 17.0, 14.3; MS (ESI) m/z 384.4 [M+H]⁺; HPLC, >99%, t_{R} 18.2 min.

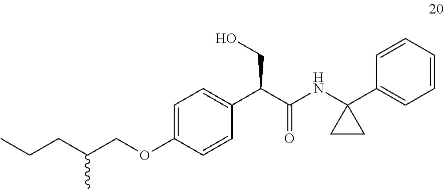 [SMILES: CCCC(C)COc1ccc([C@@H](CO)C(=O)NC2(c3ccccc3)CC2)cc1]

(2S)-3-Hydroxy-2-{4-[(2-methylpentyl)oxy]phenyl}-N-(1-phenylcyclopropyl)propenamide (20). The procedure for the synthesis of 4 was followed starting with B6 and 1-phenylcyclopropan-1-amine to afford 20 (75% yield) as a white solid: ¹H NMR (300 MHz, CDCl₃) δ 7.22-7.13 (m, 3H), 7.12-6.97 (m, 4H), 6.79 (d, J=8.7 Hz, 2H), 6.16 (s, 1H), 4.04-3.92 (m, 1H), 3.78-3.45 (m, 5H), 1.97-1.76 (m, 1H), 1.49-1.23 (m, 3H), 1.22-1.03 (m, 5H), 0.94 (d, J=6.7 Hz, 3H), 0.85 (t, J=7.0 Hz, 3H); ¹³C NMR (75 MHz, CDCl₃) δ 174.6, 159.0, 142.0, 129.4, 128.4, 128.2, 126.4, 125.1, 115.2, 73.3, 64.9, 53.4, 35.7, 35.0, 32.9, 20.0, 18.0, 17.7, 17.0, 14.3; MS (ESI) m/z 382.4 [M+H]⁺; HPLC, >97%, t_{R} 17.6 min.

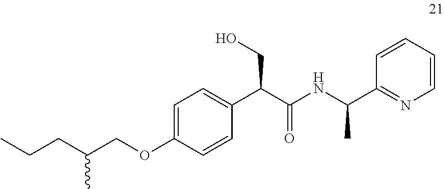 [SMILES: CCCC(C)COc1ccc([C@@H](CO)C(=O)N[C@H](C)c2ccccn2)cc1]

(2S)-3-Hydroxy-2-{4-[(2-methylpentyl)oxy]phenyl}-N-[(1 R)-1-(pyridin-2-yl)ethyl]propenamide (21). The procedure for the synthesis of 4 was followed starting with B6 and (1 R)-1-(pyridin-2-yl)ethan-1-amine to afford 21 (70% yield) as a white solid: ¹H NMR (300 MHz, CDCl₃) δ 8.34 (d, J=4.3 Hz, 1H), 7.55 (td, J=7.7, 1.8 Hz, 1H), 7.16-6.98 (m, 4H), 6.88-6.54 (m, 3H), 5.05 (p, J=6.9 Hz, 1H), 4.03 (dd, J=11.0, 8.4 Hz, 1H), 3.80-3.52 (m, 4H), 1.96-1.68 (m, 1H), 1.51-0.99 (m, 7H), 0.93 (d, J=6.7 Hz, 3H), 0.84 (t, J=7.0 Hz, 3H); ¹³C NMR (75 MHz, CDCl₃) δ 173.3, 160.7, 158.9, 148.9, 136.8, 129.5, 128.5, 122.2, 121.2, 115.0, 73.3, 65.2, 53.6, 49.9, 35.7, 32.9, 22.3, 20.0, 17.0, 14.3; MS (ESI) m/z 371.4 [M+H]⁺; HPLC, >95%, t_{R} 12.6 min.

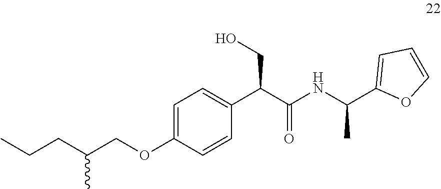 [SMILES: CCCC(C)COc1ccc([C@@H](CO)C(=O)N[C@H](C)c2ccco2)cc1]

(2S)—N-[(1 R)-1-(Furan-2-yl)ethyl]-3-hydroxy-2-{4-[(2-methylpentyl)oxy]phenyl}propenamide (22). The procedure for the synthesis of 4 was followed starting with B6 and (1 R)-1-(furan-2-yl)ethan-1-amine to afford 22 (65% yield) as a white solid: ¹H NMR (300 MHz, CDCl₃) δ 7.33-7.23 (m, 1H), 7.14 (d, J=8.6 Hz, 2H), 6.86 (d, J=8.6 Hz, 2H), 6.27 (dd, J=3.1, 1.9 Hz, 1H), 6.03 (d, J=3.2 Hz, 1H), 5.76 (d, J=8.4 Hz, 1H), 5.32-5.15 (m, 1H), 4.10 (dd, J=10.8, 8.8 Hz, 1H), 3.88-3.60 (m, 4H), 2.05-1.79 (m, 1H), 1.56-1.13 (m, 7H), 1.02 (d, J=6.7 Hz, 3H), 0.93 (t, J=7.0 Hz, 3H); ¹³C NMR (75 MHz, CDCl₃) δ 173.2, 159.0, 155.0, 141.8, 129.6, 128.1, 115.1, 110.1, 105.3, 73.3, 65.2, 53.4, 43.0, 35.7, 32.9, 20.0, 19.4, 17.0, 14.3; MS (ESI) m/z 360.4 [M+H]⁺; HPLC, >95%, t_{R} 16.5 min.

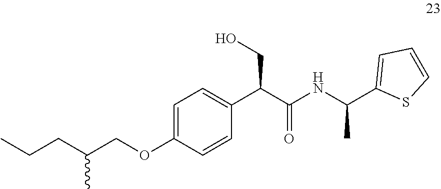 [SMILES: CCCC(C)COc1ccc([C@@H](CO)C(=O)N[C@H](C)c2cccs2)cc1]

(2S)-3-Hydroxy-2-{4-[(2-methylpentyl)oxy]phenyl}-N-[(1 R)-1-(thiophen-2-yl)ethyl]propenamide (23). The procedure for the synthesis of 4 was followed starting with B6 and (1 R)-1-(thiophen-2-yl)ethan-1-amine to afford 23 (70% yield) as a white solid: ¹H NMR (300 MHz, CDCl₃) δ 7.16-6.96 (m, 3H), 6.87-6.65 (m, 4H), 5.68 (d, J=8.2 Hz, 1H), 5.43-5.23 (m, 1H), 4.16-3.89 (m, 1H), 3.78-3.32 (m, 5H), 1.95-1.73 (m, 1H), 1.45 (d, J=6.8 Hz, 3H), 1.41-1.05 (m, 4H), 0.93 (d, J=6.7 Hz, 3H), 0.84 (t, J=7.0 Hz, 3H); ¹³C NMR (75 MHz, CDCl₃) δ 173.0, 159.0, 146.8, 129.6, 128.0, 126.7, 124.3, 123.7, 115.1, 73.3, 65.2, 53.4, 44.6, 35.7, 32.9, 22.1, 20.0, 17.0, 14.3; MS (ESI) m/z376.4 [M+H]⁺; HPLC, >99%, t_{R} 17.2 min.

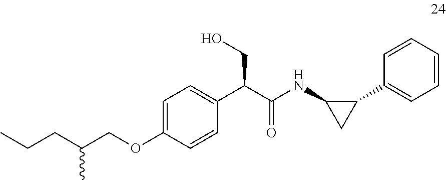 [SMILES: CCCC(C)COc1ccc([C@@H](CO)C(=O)N[C@@H]2C[C@H]2c2ccccc2)cc1]

(2S)-3-Hydroxy-2-{4-[(2-methylpentyl)oxy]phenyl}-N-[(1 R,2S)-2-phenylcyclopropyl]propenamide (24). The procedure for the synthesis of 4 was followed starting with B6 and (1 R,2S)-2-phenylcyclo propan-1-amine to afford 24 (73% yield) as a white solid: ¹H NMR (300 MHz, CDCl₃) δ 7.37-7.07 (m, 7H), 6.89 (d, J=8.6 Hz, 2H), 5.84 (br s, 1H), 4.22-4.06 (m, 1H), 3.86-3.50 (m, 4H), 2.94-2.83 (m, 1H), 2.06-1.80 (m, 2H), 1.56-1.15 (m, 6H), 1.14-1.05 (m, 1H), 1.03 (d, J=6.7 Hz, 3H), 0.94 (t, J=7.0 Hz, 3H); ¹³C NMR (75 MHz, CDCl₃) δ 175.3, 159.0, 140.2, 129.4, 128.4, 128.3, 126.4, 126.2, 115.2, 73.3, 65.1, 53.3, 35.7, 32.9, 31.9, 24.6, 20.0, 17.0, 16.2, 14.3; MS (ESI) m/z382.4 [M+H]⁺; HPLC, >95%, t_{R} 17.6 min.

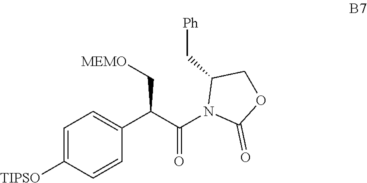

(4R)-4-Benzyl-3-[(2S)-3-[(2-methoxyethoxy)methoxy]-2-(4-{[tris(propan-2-yl)silyl]oxy}phenyl)propanoyl]-1,3-oxazolidin-2-one (B7). To a solution of B5b in DCM (10 mL) were added DIPEA (210 μL, 1.21 mmol), DMAP (10 mg, 0.08 mmol) and MEM chloride (69 μL, 0.06 mmol), and the resulting reaction was stirred at room temperature overnight. The reaction was diluted with H₂O (20 mL). Layers were separated and the aqueous layer was extracted with additional DCM (3×20 mL). The combined organic layers were washed with brine (2×20 mL), dried (Na₂SO₄), and concentrated under reduced pressure. The residue was subjected to chromatography on silica gel using 0-100% EtOAc in hexanes to afford B7 (242 mg, quantitative yield) as a colorless film: ¹H NMR (300 MHz, CDCl₃) δ 7.39-7.11 (m, 7H), 6.80 (d, J=8.5 Hz, 2H), 5.37 (dd, J=10.2, 4.7 Hz, 1H), 4.83-4.70 (m, 2H), 4.70-4.55 (m, 1H), 4.30 (t, J=9.8 Hz, 1H), 4.15-3.96 (m, 2H), 3.78-3.63 (m, 3H), 3.60-3.50 (m, 2H), 3.38 (s, 3H), 3.26 (dd, J=13.5, 2.8 Hz, 1H), 2.99-2.72 (m, 1H), 1.36-1.14 (m, 3H), 1.14-0.94 (m, 18H); MS (ESI) m/z 608.4 [M+Na]⁺.

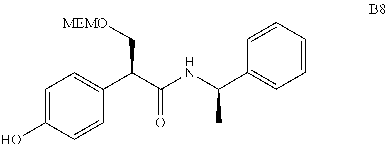

(2S)-2-(4-Hydroxyphenyl)-3-[(2-methoxyethoxy)methoxy]-N-[(1 R)-1-phenylethyl]propenamide (B8). The oxazolidinone auxiliary in B7 was removed following the procedure for the synthesis of B6 to afford (2S)-3-[(2-methoxyethoxy)methoxy]-2-(4-{[tris(propan-2-yl)silyl]oxy}phenyl)propanoic acid (99% yield) as a colorless oil: MS (ESI) m/z 449.6 [M+Na]⁺. Coupling of the acid with (R)-(+)-α-methylbenzylamine was carried out following the procedure for the synthesis of 4 to give (2S)-3-[(2-methoxyethoxy)methoxy]-N-[(1 R)-1-phenylethyl]-2-(4-{[tris(propan-2-yl)silyl]oxy}phenyl)propanamide (33% yield) as a colorless oil: MS (ESI) m/z 530.8 [M+H]⁺. To a solution of the above amide (570 mg, 1.08 mmol) in THE (20 mL) at 0° C. was added TBAF (1.0 M in THF, 1.61 mL, 1.61 mmol), and the resulting reaction was stirred at 0° C. for 2 h. The reaction was quenched with H₂O (20 mL) and extracted with EtOAc (2×20 mL). The combined organic layers were washed with brine (2×30 mL), dried (Na₂SO₄), and concentrated under reduced pressure. The residue was subjected to chromatography on silica gel using 0-100% EtOAc in hexanes to afford B8 (380 mg, 94% yield) as a colorless residue: ¹H NMR (300 MHz, CD₃OD) δ 7.26-7.04 (m, 7H), 6.74 (d, J=8.5 Hz, 2H), 5.12-4.92 (m, 4H), 4.82-4.56 (m, 2H), 4.14 (t, J=9.6 Hz, 1H), 3.84 (dd, J=9.6, 5.1 Hz, 1H), 3.77-3.60 (m, 3H), 3.52 (dd, J=5.8, 3.3 Hz, 2H), 1.43 (d, J=7.0 Hz, 3H); MS (ESI) m/z 374.0 [M+H]⁺.

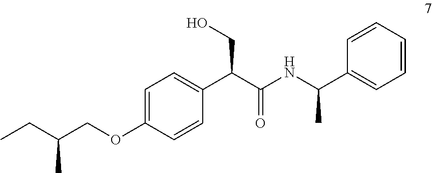 [SMILES: CC[C@H](C)COc1ccc([C@@H](CO)C(=O)N[C@H](C)c2ccccc2)cc1]

(2S)-3-Hydroxy-2-{4-[(2S)-2-methylbutoxy]phenyl}-N-[(1 R)-1-phenylethyl]propenamide (7). A mixture of B8 (100 mg, 0.27 mmol), (2S)-2-methylbutyl 4-methylbenzene-1-sulfonate (97 mg, 0.41 mmol), and K₂CO₃ (111 mg, 0.80 mmol) in MeCN (3 mL) was heated overnight at 90° C. in a sealed vessel. The reaction was cooled to room temperature and diluted with EtOAc (5 mL) and H₂O (5 mL). The layers were separated, and the aqueous layer was extracted with additional EtOAc (2×5 mL). The combined organic layers were washed with brine (2×10 mL), dried (Na₂SO₄), and concentrated under reduced procedure. The residue was subjected to chromatography on silica gel using 0-100% EtOAc in hexanes to afford (2S)-3-[(2-methoxyethoxy)methoxy]-2-{4-[(2S)-2-methylbutoxy]phenyl}-N-[(1 R)-1-phenylethyl]-propanamide (82 mg, 69% yield) as a colorless oil: MS (ESI) m/z 444.6 [M+H]⁺. To a solution of the above ether (71 mg, 0.16 mmol) in DCM (10 mL) was added ZnBr₂ (61 mg, excess), and the resulting reaction was stirred at room temperature overnight. The reaction was diluted with DCM (5 mL) and washed with brine (2×10 mL), dried (Na₂SO₄), and concentrated under reduced pressure. The residue was subjected to chromatography using 12% isopropyl amine (IPA) in hexanes on a ChiralPak IA (5 μm) column to afford 7 (20 mg, 35% yield) as a white solid: ¹H NMR (300 MHz, CDCl₃) δ 7.33-7.17 (m, 3H), 7.16-7.03 (m, 4H), 6.91-6.79 (m, 2H), 5.74 (d, J=7.4 Hz, 1H), 5.10 (p, J=7.0 Hz, 1H), 4.13-4.00 (m, 1H), 3.85-3.76 (m, 1H), 3.76-3.59 (m, 3H), 3.57-3.47 (m, 1H), 1.93-1.77 (m, 1H), 1.65-1.48 (m, 1H), 1.45-1.35 (m, 3H), 1.35-1.12 (m, 1H), 1.08-0.98 (m, 3H), 0.98-0.88 (m, 3H); ¹³C NMR (75 MHz, CDCl₃) δ 173.2, 159.0, 143.1, 129.5, 128.6, 128.3, 127.4, 125.8, 115.1, 73.0, 65.2, 53.4, 48.8, 34.7, 26.1, 22.0, 16.5, 11.3; MS (ESI) m/z 356.6 [M+H]⁺; HPLC, >99%, t_{R} 6.2 min (12% IPA/hexane; 5 μm ChiralPak IA column).

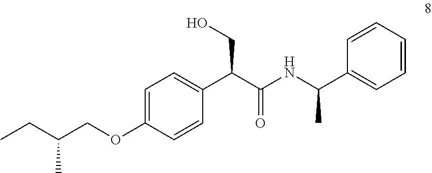 [SMILES: CC[C@@H](C)COc1ccc([C@@H](CO)C(=O)N[C@H](C)c2ccccc2)cc1]

(2S)-3-Hydroxy-2-{4-[(2R)-2-methylbutoxy]phenyl}-N-[(1 R)-1-phenylethyl]propenamide (8). The procedure for the synthesis of 7 was followed starting with (2R)-2-methylbutyl 4-methylbenzene-1-sulfonate to afford 8 (33% yield) as a white solid: ¹H NMR (300 MHz, CDCl₃) δ 7.36-7.20 (m, 3H), 7.18-7.05 (m, 4H), 6.93-6.82 (m, 2H), 5.74 (d, J=7.6 Hz, 1H), 5.12 (p, J=7.1 Hz, 1H), 4.16-3.99 (m, 1H), 3.87-3.78 (m, 1H), 3.78-3.59 (m, 3H), 3.55 (dd, J=8.8, 4.2 Hz, 1H), 1.95-1.77 (m, 1H), 1.65-1.48 (m, 1H), 1.43 (d, J=7.0 Hz, 3H), 1.37-1.17 (m, 1H), 1.03 (d, J=6.7 Hz, 3H), 0.97 (t, J=7.4 Hz, 3H); ¹³C NMR (75 MHz, CDCl₃) δ 173.2, 159.0, 143.1, 129.5, 128.6, 128.3, 127.2, 125.8, 115.1, 73.0, 65.2, 53.4, 48.8, 34.7, 26.1, 22.0, 16.5, 11.3; MS (ESI) m/z356.4 [M+H]⁺; HPLC, >99%, t_{R} 6.3 min (12% IPA/hexane; 5 μm ChiralPak IA column).

### Synthesis of Compounds 25-27 and 30-37 (Scheme C)

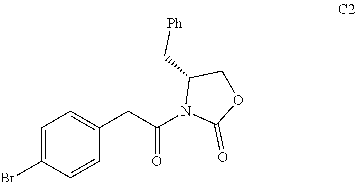 [SMILES: O=C(Cc1ccc(Br)cc1)N1C(=O)OC[C@H]1Cc1ccccc1]

(4R)-4-Benzyl-3-[2-(4-bromophenyl)acetyl]-1,3-oxazolidin-2-one (C2). The procedure for the synthesis of B4a was followed starting with 2-(4-bromophenyl)acetyl chloride (C1) to afford C2 (49% yield) as a white solid: ¹H NMR (300 MHz, CDCl₃) δ 7.48 (d, J=8.4 Hz, 2H), 7.34-7.25 (m, 3H), 7.22 (d, J=8.4 Hz, 2H), 7.13 (dd, J=7.5, 2.0 Hz, 2H), 4.73-4.58 (m, 1H), 4.30 (d, J=15.8 Hz, 1H), 4.25-4.11 (m, 3H), 3.26 (dd, J=13.4, 3.4 Hz, 1H), 2.76 (dd, J=13.4, 9.5 Hz, 1H); ¹³C NMR (75 MHz, CDCl₃) δ 170.7, 153.4, 135.0, 132.5, 131.7, 131.5, 129.4, 129.0, 127.4, 121.4, 66.2, 55.3, 41.0, 37.7; MS (ESI) m/z 374.2 [M+H]⁺ (⁷⁹Br), 376.2 [M+H]⁺ (⁸¹Br).

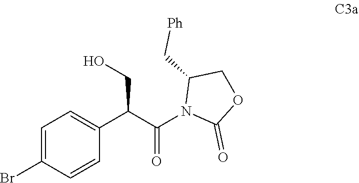 [SMILES: O=C1OC[C@@H](Cc2ccccc2)N1C(=O)[C@H](CO)c1ccc(Br)cc1]

(4R)-4-Benzyl-3-[(2S)-2-(4-bromophenyl)-3-hydroxypropanoyl]-1,3-oxazolidin-2-one (C3a). The procedure for the synthesis of B5a was followed starting with C2 to afford C3a (54% yield) as a white solid: ¹H NMR (300 MHz, CDCl₃) δ 7.45 (d, J=8.5 Hz, 2H), 7.38-7.18 (m, 7H), 5.19 (dd, J=8.9, 5.0 Hz, 1H), 4.66 (ddt, J=12.4, 6.6, 3.1 Hz, 1H), 4.20 (ddd, J=9.2, 7.9, 5.9 Hz, 1H), 4.15-4.04 (m, 2H), 3.86 (ddd, J=11.2, 6.3, 5.0 Hz, 1H), 3.33 (dd, J=13.4, 3.5 Hz, 1H), 2.96-2.77 (m, 1H), 2.16 (t, J=6.6 Hz, 1H); MS (ESI) m/z 426.0 [M+Na]⁺ (⁷⁹Br), 428.0 [M+Na]⁺ (⁸¹Br).

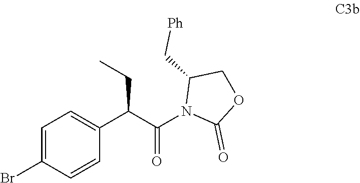 [SMILES: CC[C@@H](C(=O)N1C(=O)OC[C@H]1Cc1ccccc1)c1ccc(Br)cc1]

(4R)-4-Benzyl-3-[(2R)-2-(4-bromophenyl)butanoyl]-1,3-oxazolidin-2-one (C3b). To a solution of C2 (1.0 g, 2.67 mmol) in THE (30 mL) at 0° C. was slowly added NaHMDS (1.0 M in THF, 2.94 mL, 2.94 mmol), and the resulting reaction was stirred at 0° C. for 30 min. Afterwards, iodoethane (1.07 mL, 13.36 mmol) was slowly added at 0° C., and the reaction was warmed to room temperature and stirred overnight. The reaction was cooled to 0° C. and quenched with saturated aqueous NH₄Cl (20 mL). The layers were separated, and the aqueous layer was extracted with EtOAc (3×30 mL). The combined organic layers were washed with brine (3×20 mL), dried (Na₂SO₄), and concentrated under reduced pressure. The residue was subjected to chromatography on silica gel using 0-100% EtOAc in hexanes to afford C3b (584 mg, 55% yield) as a light green oil: MS (ESI) m/z 402.2 [M+H]⁺ (⁷⁹Br), 404.4 [M+H]⁺ (⁸¹Br).

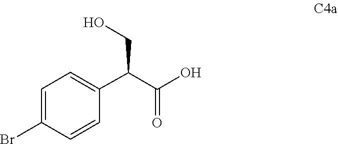 [SMILES: O=C(O)[C@H](CO)c1ccc(Br)cc1]

(2S)-2-(4-Bromophenyl)-3-hydroxypropanoic acid (C4a). The procedure for the synthesis of B6 was followed starting with C3a to afford C4a (61% yield) as a white solid: ¹H NMR (300 MHz, CD₃OD) δ 7.48 (d, J=8.5 Hz, 2H), 7.26 (d, J=8.4 Hz, 2H), 4.10-3.98 (m, 1H), 3.78-3.67 (m, 2H). ¹³C NMR (75 MHz, CD₃OD) δ 175.5, 137.4, 132.7, 131.3, 122.3, 64.8, 55.2.

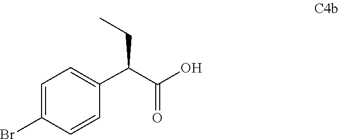

(2R)-2-(4-Bromophenyl)butanoic acid (C4b). The procedure for the synthesis of B6 was followed starting with C3b to afford C4b (60% yield) as a white solid: MS (ESI) m/z 240.8 [M−H]⁻ (⁷⁹Br), 242.8 [M−H]⁻ (⁸¹Br). This compound was used for the next transformation without further characterization.

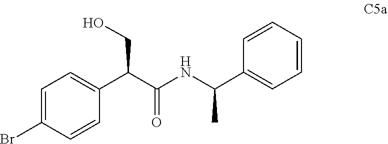 [SMILES: C[C@@H](NC(=O)[C@H](CO)c1ccc(Br)cc1)c1ccccc1]

(2S)-2-(4-Bromophenyl)-3-hydroxy-N-[(1 R)-1-phenylethyl]propenamide (C5a). To a mixture of C4a (49 mg, 0.20 mmol) and (R)-(+)-α-methylbenzylamine (29 mg, 0.24 mmol) in CH₃CN (3 mL) at room temperature were added TCFH (62 mg, 0.22 mmol) and NMI (34 mg, 0.42 mmol) successively, and the resulting reaction was stirred at room temperature for 3 h. The solvent was evaporated, and the residue was subjected to chromatography on silica gel using 0-100% EtOAc in hexanes to afford C₅a (48 mg, 68%) as a white solid: ¹H NMR (300 MHz, CDCl₃) δ 7.45 (d, J=8.4 Hz, 2H), 7.33-7.22 (m, 3H), 7.16-7.04 (m, 4H), 5.72 (d, J=7.5 Hz, 1H), 5.11 (p, J=7.1 Hz, 1H), 4.12-3.93 (m, 1H), 3.87-3.70 (m, 1H), 3.63 (dd, J=8.4, 4.2 Hz, 1H), 3.27 (dd, J=8.2, 4.9 Hz, 1H), 1.44 (d, J=6.9 Hz, 3H). ¹³C NMR (75 MHz, CDCl₃) δ 171.8, 142.8, 135.6, 132.2, 130.1, 128.6, 127.4, 125.8, 121.9, 64.9, 53.7, 49.1, 21.9; MS (ESI) m/z 348.0 [M+H]⁺ (⁷⁹Br), 340.0 [M+H]⁺ (⁸¹Br).

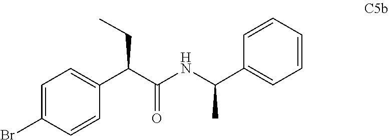 [SMILES: CC[C@@H](C(=O)N[C@H](C)c1ccccc1)c1ccc(Br)cc1]

(2R)-2-(4-Bromophenyl)-N-[(1 R)-1-phenylethyl]butanamide (C5b). The procedure for the synthesis of C5a was followed starting with C4b to furnish amide C5b (64% yield) as a white solid: MS (ESI) m/z 346.2 [M+H]⁺ (⁷⁹Br), 348.2 [M+H]⁺ (⁸¹Br).

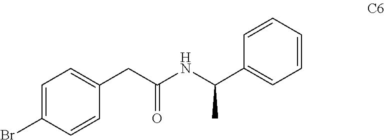 [SMILES: C[C@@H](NC(=O)Cc1ccc(Br)cc1)c1ccccc1]

2-(4-Bromophenyl)-N-[(1 R)-1-phenylethyl]acetamide (C6). To a solution of 4-bromophenylacetyl chloride (C1) (234 mg, 1 mmol) in DCM (10 mL) at room temperature were added (R)-(+)-α-methylbenzylamine (145 mg, 1.2 mmol) and Et₃N (279 μL, 2 mmol), and the reaction was stirred at room temperature for 1 h. The solvent was removed under reduced pressure. The residue was subjected to chromatography on silica gel using 0-100% EtOAc in hexanes to afford C₆ (240 mg, 75% yield) as white solid: ¹H NMR (300 MHz, CDCl₃) δ 7.46 (d, J=8.3 Hz, 2H), 7.39-7.17 (m, 5H), 7.12 (d, J=8.3 Hz, 2H), 5.70 (d, J=6.2 Hz, 1H), 5.10 (p, J=7.0 Hz, 1H), 3.49 (s, 2H), 1.42 (d, J=6.9 Hz, 3H); MS (ESI) m/z 318.2 [M+H]⁺ (⁷⁹Br), 320.2 [M+H]⁺ (⁸¹Br).

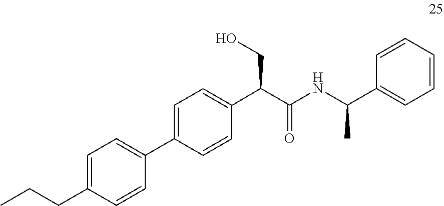 [SMILES: CCCc1ccc(-c2ccc([C@@H](CO)C(=O)N[C@H](C)c3ccccc3)cc2)cc1]

(2S)-3-Hydroxy-N-[(1 R)-1-phenylethyl]-2-{4′-propyl-[1,1′-biphenyl]-4-yl}propenamide (25). A mixture of C₅a (100 mg, 0.26 mmol), 4-propylphenylboronic acid (65 mg, 0.40 mmol), Pd(dppf)Cl₂·CH₂Cl₂ (22 mg, 0.03 mmol) and K₃PO₄ (169 mg, 0.80 mmol) in dimethoxyethane (6 mL) and H₂O (1.5 mL) was heated under microwave irradiation at 160° C. for 6 min. The resulting mixture was poured into cold 1 N NaOH (15 mL) and extracted with DCM (3×20 mL). The combined organic layers were dried (Na₂SO₄) and concentrated under reduced pressure. The residue was subjected to chromatography on silica gel using 0-100% EtOAc in hexanes to afford 25 (83 mg, 75% yield) as an off-white solid: ¹H NMR (300 MHz, CDCl₃) δ 7.68-7.40 (m, 4H), 7.36-7.19 (m, 7H), 7.18-7.09 (m, 2H), 5.79 (d, J=7.7 Hz, 1H), 5.16 (p, J=7.0 Hz, 1H), 4.29-4.05 (m, 1H), 3.94-3.65 (m, 2H), 3.50 (br s, 1H), 2.76-2.53 (m, 2H), 1.70 (td, J=7.5, 3.8 Hz, 2H), 1.46 (d, J=6.9 Hz, 3H), 1.00 (t, J=7.3 Hz, 3H); ¹³C NMR (75 MHz, CDCl₃) δ 172.7, 143.0, 142.2, 140.8, 137.7, 135.2, 129.0, 128.9, 128.6, 127.6, 127.3, 126.8, 125.8, 65.1, 53.9, 48.9, 37.7, 24.5, 22.0, 13.8; MS (ESI) m/z388.4 [M+H]⁺; HPLC, >99%, t_{R} 17.4 min.

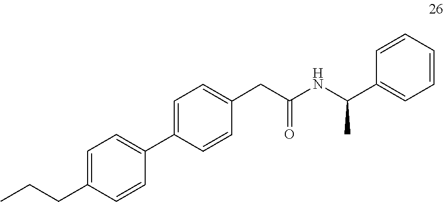 [SMILES: CCCc1ccc(-c2ccc(CC(=O)N[C@H](C)c3ccccc3)cc2)cc1]

N-[(1 R)-1-Phenylethyl]-2-{4′-propyl-[1,1′-biphenyl]-4-yl}acetamide (26). The procedure for the synthesis of 25 was followed starting with C₆ to afford 26 (99% yield) as a white solid: ¹H NMR (300 MHz, CDCl₃) δ 7.58-7.46 (m, 4H), 7.34-7.14 (m, 9H), 5.91 (d, J=7.5 Hz, 1H), 5.12 (p, J=7.0 Hz, 1H), 3.57 (s, 2H), 2.72-2.45 (m, 2H), 1.78-1.58 (m, 2H), 1.40 (d, J=6.9 Hz, 3H), 0.97 (t, J=7.3 Hz, 3H); ¹³C NMR (75 MHz, CDCl₃) δ 170.1, 143.2, 142.0, 140.1, 137.9, 133.7, 129.7, 129.0, 128.6, 127.5, 127.3, 126.8, 126.0, 48.8, 43.4, 37.7, 24.5, 21.8, 13.9; MS (ESI) m/z 358.4 [M+H]⁺; HPLC, >98%, t_{R} 18.8 min.

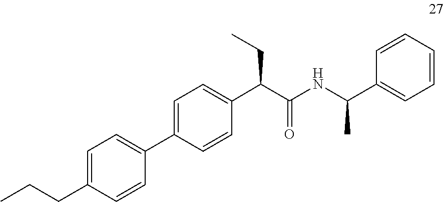 [SMILES: CCCc1ccc(-c2ccc([C@@H](CC)C(=O)N[C@H](C)c3ccccc3)cc2)cc1]

(2R)—N-[(1 R)-1-Phenylethyl]-2-{4′-propyl-[1,1′-biphenyl]-4-yl}butanamide (27). The procedure for the synthesis of 25 was followed starting with C5b to afford 27 (80% yield) as a white solid: ¹H NMR (300 MHz, CDCl₃) δ 7.56 (t, J=7.7 Hz, 4H), 7.43-7.21 (m, 7H), 7.18-7.07 (m, 2H), 5.84 (d, J=7.9 Hz, 1H), 5.15 (p, J=7.0 Hz, 1H), 3.34 (t, J=7.6 Hz, 1H), 2.83-2.55 (m, 2H), 2.40-2.17 (m, 1H), 1.94-1.79 (m, 1H), 1.79-1.62 (m, 2H), 1.47 (d, J=6.9 Hz, 3H), 1.11-0.87 (m, 6H); ¹³C NMR (75 MHz, CDCl₃) δ 172.6, 143.2, 141.9, 140.0, 138.7, 138.1, 128.9, 128.5, 128.4, 127.2, 127.1, 126.8, 125.9, 54.9, 48.7, 37.7, 26.4, 24.5, 21.9, 13.9, 12.4; MS (ESI) m/z 386.6 [M+H]⁺; HPLC, >99%, t_{R} 19.6 min.

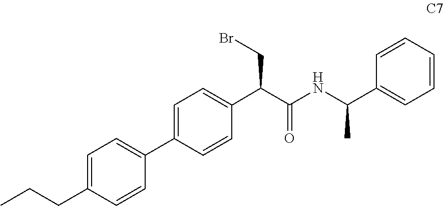 [SMILES: CCCc1ccc(-c2ccc([C@@H](CBr)C(=O)N[C@H](C)c3ccccc3)cc2)cc1]

(2R)-3-Bromo-N-[(1 R)-1-phenylethyl]-2-{4′-propyl-[1,1′-biphenyl]-4-yl}propenamide (C7). A solution of 25 (5 mg, 0.01 mmol), CBr₄ (6.4 mg, 0.02 mmol), and PPh₃ (5.1 mg, 0.02 mmol) in THE (1 mL) was stirred at room temperature for 2 h. The reaction was diluted with DCM (5 mL) and washed with H₂O (10 mL). The layers were separated, and the organic layer was washed with brine (2×5 mL), dried (Na₂SO₄), and concentrated under reduced pressure. The residue was subjected to chromatography on silica gel using 0-100% EtOAc in hexanes to afford C₇ (3 mg, 52% yield) as an off-white solid: ¹H NMR (300 MHz, CDCl₃) δ 7.50 (dd, J=12.8, 8.2 Hz, 4H), 7.37-7.28 (m, 2H), 7.28-7.14 (m, 5H), 7.09 (dd, J=7.3, 1.8 Hz, 2H), 5.97 (d, J=7.8 Hz, 1H), 5.12 (p, J=7.0 Hz, 1H), 4.09 (t, J=9.4 Hz, 1H), 3.81 (dd, J=8.9, 5.8 Hz, 1H), 3.55 (dd, J=9.8, 5.8 Hz, 1H), 2.77-2.54 (m, 2H), 1.67 (dd, J=15.0, 7.4 Hz, 2H), 1.47 (d, J=6.9 Hz, 3H), 0.97 (t, J=7.3 Hz, 3H); MS (ESI) m/z450.2 [M+H]⁺ (⁷⁹Br), 452.2 [M+H]⁺ (⁸¹Br).

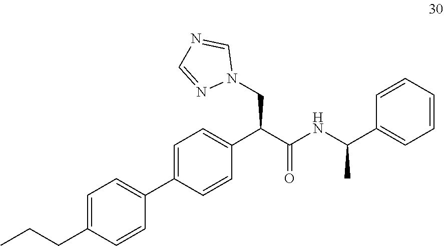 [SMILES: CCCc1ccc(-c2ccc([C@@H](Cn3cncn3)C(=O)N[C@H](C)c3ccccc3)cc2)cc1]

(2S)—N-[(1 R)-1-Phenylethyl]-2-{4′-propyl-[1,1′-biphenyl]-4-yl}-3-(4H-1,2,4-triazol-4-yl)propenamide (30). A solution of C7 (15 mg, 0.03 mmol), 1,2,4-triazole (3.4 mg, 0.05 mmol), and K₂CO₃ (13.8 mg, 0.1 mmol) in DMF (1 mL) was stirred at room temperature overnight. The reaction was diluted with EtOAc (5 mL) and H₂O (5 mL). Layers were separated and the aqueous layer was extracted with additional EtOAc (2×5 mL). The combined organic layers were washed with brine (2×10 mL), dried (Na₂SO₄), and concentrated under reduced pressure. The residue was subjected to chromatography on silica gel using 0-100% EtOAc in hexanes to afford 30 (3 mg, 20% yield) as a white solid:¹H NMR (300 MHz,) 6 8.30 (s, 1H), 8.02 (s, 1H), 7.66-7.46 (m, 5H), 7.37 (d, J=8.2 Hz, 1H), 7.27 (d, J=8.0 Hz, 3H), 7.16 (d, J=7.0 Hz, 2H), 7.09-7.01 (m, 2H), 4.57-4.25 (m, 4H), 2.64 (t, J=7.4 Hz, 2H), 1.78-1.54 (m, 2H), 1.40-1.21 (m, 4H), 0.98 (t, J=7.3 Hz, 3H); ¹³C NMR (75 MHz, CD₃OD) δ 172.1, 152.3, 145.7, 130.0, 129.4, 129.4, 129.3, 128.2, 128.1, 128.2, 128.0, 127.9, 127.9, 126.8, 126.7, 52.9, 52.7, 38.6, 25.7, 22.2, 22.0, 14.1; MS (ESI) m/z 439.6 [M+H]⁺; HPLC, >95%, t_{R} 17.9 min.

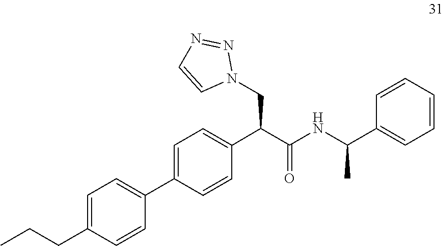 [SMILES: CCCc1ccc(-c2ccc([C@@H](Cn3ccnn3)C(=O)N[C@H](C)c3ccccc3)cc2)cc1]

(2S)—N-[(1 R)-1-Phenylethyl]-2-{4′-propyl-[1,1′-biphenyl]-4-yl}-3-(1H-1,2,3-triazol-1-yl)propenamide (31). The procedure for the synthesis of 30 was followed starting with 1,2,3-triazole to afford 31 (25% yield) as a white solid. ¹H NMR (300 MHz, CDCl₃) δ 7.59-7.42 (m, 5H), 7.40-7.13 (m, 10H), 5.72 (d, J=7.8 Hz, 1H), 5.20 (dd, J=13.5, 8.0 Hz, 1H), 5.09 (p, J=7.1 Hz, 1H), 4.75 (dd, J=13.6, 7.4 Hz, 1H), 4.26 (t, J=7.6 Hz, 1H), 2.72-2.49 (m, 2H), 1.78-1.62 (m, 2H), 1.38 (d, J=6.9 Hz, 3H), 0.97 (t, J=7.3 Hz, 3H); ¹³C NMR (75 MHz, CDCl₃) δ 169.6, 142.7, 142.1, 140.9, 138.5, 137.7, 134.8, 134.0, 128.9, 128.6, 128.4, 127.4, 127.3, 126.8, 126.1, 56.9, 52.5, 48.9, 37.7, 24.5, 21.2, 13.8; MS (ESI) m/z439.6 [M+H]⁺; HPLC, >99%, t_{R}18.8 min.

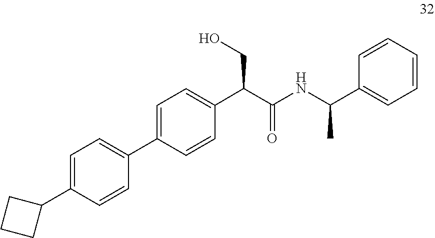 [SMILES: C[C@@H](NC(=O)[C@H](CO)c1ccc(-c2ccc(C3CCC3)cc2)cc1)c1ccccc1]

(2S)-2-{4′-Cyclobutyl-[1,1′-biphenyl]-4-yl}-3-hydroxy-N-[(1 R)-1-phenylethyl]propenamide (32). The procedure for the synthesis of 25 was followed starting with C₅a and 4-cyclobutyl phenylboronic acid to afford 32 (65% yield) as an off-white solid: ¹H NMR (300 MHz, CDCl₃) δ 7.64-7.45 (m, 4H), 7.42-7.21 (m, 7H), 7.14 (ddd, J=7.8, 5.7, 1.4 Hz, 2H), 5.77 (d, J=7.9 Hz, 1H), 5.13 (p, J=7.1 Hz, 1H), 4.19-4.09 (m, 1H), 3.80 (dd, J=11.0, 4.1 Hz, 1H), 3.73 (dd, J=8.5, 4.2 Hz, 1H), 3.59 (p, J=8.7 Hz, 1H), 3.47 (s, 1H), 2.48-2.27 (m, 2H), 2.29-2.11 (m, 2H), 2.10-1.95 (m, 1H), 1.95-1.74 (m, 1H), 1.43 (d, J=6.9 Hz, 3H); ¹³C NMR (75 MHz, CDCl₃) δ 172.7, 145.8, 143.0, 140.8, 137.8, 135.3, 129.1, 128.9, 128.6, 127.6, 127.3, 126.8, 125.8, 65.1, 54.0, 49.0, 40.1, 29.8, 22.0, 18.3; MS(ESI) m/z 400.2 [M+H]⁺; HPLC, >99%, t_{R} 18.3 min.

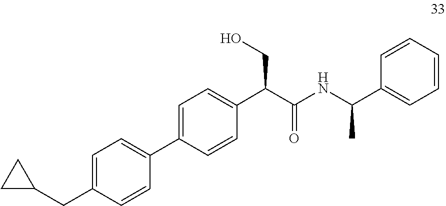 [SMILES: C[C@@H](NC(=O)[C@H](CO)c1ccc(-c2ccc(CC3CC3)cc2)cc1)c1ccccc1]

(2S)-2-[4′-(Cyclopropylmethyl)-[1,1′-biphenyl]-4-yl]-3-hydroxy-N-[(1 R)-1-phenylethyl]propenamide (33). The procedure for the synthesis of 25 was followed starting with C5a and 4-cyclopropylmethyl phenylboronic acid to afford 33 (30% yield) as an off-white solid: ¹H NMR (300 MHz, CDCl₃) δ 7.55 (d, J=8.2 Hz, 2H), 7.51 (d, J=8.2 Hz, 2H), 7.38-7.32 (m, 2H), 7.30-7.21 (m, 5H), 7.16-7.08 (m, 2H), 5.78 (d, J=7.9 Hz, 1H), 5.13 (p, J=7.1 Hz, 1H), 4.14 (ddd, J=13.1, 7.0, 3.5 Hz, 1H), 3.88-3.64 (m, 2H), 3.47 (dd, J=8.5, 4.5 Hz, 1H), 2.60 (d, J=6.9 Hz, 2H), 1.44 (d, J=6.9 Hz, 3H), 1.10-0.93 (m, 1H), 0.64-0.48 (m, 2H), 0.29-0.17 (m, 2H); ¹³C NMR (75 MHz, CDCl₃) δ 172.7, 143.0, 141.7, 140.8, 137.9, 135.3, 128.9, 128.9, 128.6, 127.6, 127.3, 126.9, 125.8, 65.1, 53.9, 48.9, 40.0, 22.0, 11.8, 4.7; MS (ESI) m/z 400.2 [M+H]⁺; HPLC, >99%, t_{R} 17.1 min.

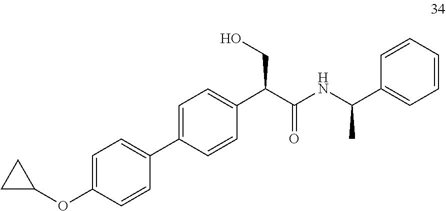 [SMILES: C[C@@H](NC(=O)[C@H](CO)c1ccc(-c2ccc(OC3CC3)cc2)cc1)c1ccccc1]

(2S)-2-{4′-cyclopropoxy-[1,1′-biphenyl]-4-yl}-3-hydroxy-N-[(1 R)-1-phenylethyl]propenamide (34). The procedure for the synthesis of 25 was followed starting with C5a and (4-cyclopropyloxyphenyl) boronic acid to afford 34 (33% yield) as an off-white solid: ¹H NMR (300 MHz, CDCl₃) δ 7.59-7.43 (m, 4H), 7.34-7.21 (m, 5H), 7.18-7.05 (m, 4H), 5.78 (d, J=7.9 Hz, 1H), 5.13 (p, J=7.1 Hz, 1H), 4.13 (dd, J=11.0, 8.5 Hz, 1H), 3.89-3.61 (m, 3H), 2.80 (s, 1H), 1.43 (d, J=6.9 Hz, 3H), 0.88-0.70 (m, 4H); ¹³C NMR (75 MHz, CDCl₃) δ 172.7, 158.7, 143.0, 140.5, 134.9, 133.2, 128.9, 128.6, 127.9, 127.3, 125.8, 115.4, 65.1, 53.9, 50.9, 48.9, 22.0, 6.2; MS (ESI) m/z 402.2 [M+H]⁺; HPLC, >99%, t_{R} 15.8 min.

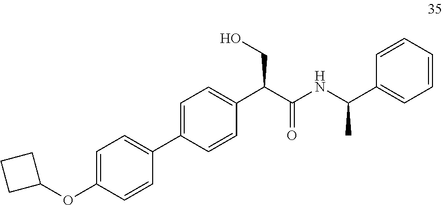 [SMILES: C[C@@H](NC(=O)[C@H](CO)c1ccc(-c2ccc(OC3CCC3)cc2)cc1)c1ccccc1]

(2S)-2-{4′-Cyclobutoxy-[1,1′-biphenyl]-4-yl}-3-hydroxy-N-[(1 R)-1-phenylethyl]propenamide (35). The procedure for the synthesis of 25 was followed starting with C₅a and (4-cyclobutyloxyphenyl)boronic acid to afford 35 (43% yield) as a white solid: ¹H NMR (300 MHz, CDCl₃ δ 7.55-7.37 (m, 4H), 7.33-7.19 (m, 5H), 7.13 (dd, J=8.0, 1.7 Hz, 2H), 6.89 (d, J=8.7 Hz, 2H), 5.75 (d, J=8.0 Hz, 1H), 5.13 (p, J=7.2 Hz, 1H), 4.68 (p, J=7.2 Hz, 1H), 4.13 (dd, J=11.0, 8.6 Hz, 1H), 3.79 (dd, J=10.9, 4.5 Hz, 1H), 3.72 (dd, J=8.6, 4.2 Hz, 1H), 3.47 (s, 1H), 2.58-2.38 (m, 2H), 2.20 (dtd, J=12.6, 9.9, 7.8 Hz, 2H), 2.03-1.80 (m, 1H), 1.79-1.65 (m, 1H), 1.43 (d, J=7.0 Hz, 3H);¹³C NMR (75 MHz, CDCl₃) δ 172.7, 157.4, 142.9, 140.5, 134.8, 132.7, 128.9, 128.6, 127.6, 127.3, 127.3, 125.8, 115.3, 71.6, 65.1, 53.9, 48.9, 30.7, 22.0, 13.3. MS (ESI) m/z 416.2 [M+H]⁺; HPLC, >99%, t_{R} 17.2 min.

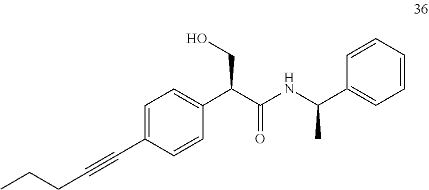 [SMILES: CCCC#Cc1ccc([C@@H](CO)C(=O)N[C@H](C)c2ccccc2)cc1]

(2S)-3-Hydroxy-2-[4-(pent-1-yn-1-yl)phenyl]-N-[(1 R)-1-phenylethyl]propenamide (36). A mixture of C₅a (40 mg, 0.11 mmol), tri-o-tolylphosphine (7 mg, 0.02 mmol), Pd(OAc)₂ (2.6 mg, 0.01 mmol), Et₃N (48 μL, 0.34 mmol), 1-pentyne (12.5 μL, 0.12 mmol) in MeCN (2 mL) was heated at 75° C. in a sealed vessel for 4 h. The reaction was cooled to room temperature and quenched with H₂O (5 mL) and extracted with EtOAc (2×10 mL). The combined organic layers were washed with brine (2×10 mL), dried (Na₂SO₄), and concentrated under reduced pressure. The residue was subjected to chromatography on silica gel using 0-100% EtOAc in hexanes to afford 36 (31 mg, 80% yield) as a brownish solid: ¹H NMR (300 MHz, CDCl₃) δ 7.36 (d, J=8.2 Hz, 2H), 7.32-7.21 (m, 3H), 7.15-7.07 (m, 4H), 5.65 (d, J=7.4 Hz, 1H), 5.10 (p, J=7.1 Hz, 1H), 4.18-3.98 (m, 1H), 3.83-3.59 (m, 2H), 3.38 (dd, J=8.5, 4.5 Hz, 1H), 2.39 (t, J=7.0 Hz, 2H), 1.75-1.59 (m, 2H), 1.42 (d, J=6.9 Hz, 3H), 1.05 (t, J=7.4 Hz, 3H); ¹³C NMR (75 MHz, CDCl₃) δ 172.3, 142.9, 135.8, 132.2, 128.7, 128.4, 127.4, 125.8, 123.9, 91.2, 80.1, 65.0, 54.1, 49.0, 22.2, 22.0, 21.4, 13.5; MS (ESI) m/z 436.4 [M+H]⁺; HPLC, >99%, t_{R} 15.7 min.

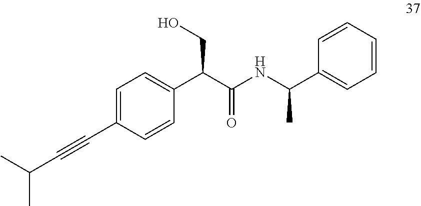 [SMILES: CC(C)C#Cc1ccc([C@@H](CO)C(=O)N[C@H](C)c2ccccc2)cc1]

(2S)-3-Hydroxy-2-[4-(3-methylbut-1-yn-1-yl)phenyl]-N-[(1 R)-1-phenylethyl]propenamide (37). The procedure for the synthesis of 36 was followed starting with 3-methylbut-1-yne to afford 37 (27% yield) as a white solid: ¹H NMR (300 MHz, CDCl₃) δ 7.36 (d, J=8.0 Hz, 2H), 7.31-7.17 (m, 3H), 7.16-7.01 (m, 4H), 5.72 (d, J=7.8 Hz, 1H), 5.09 (p, J=7.1 Hz, 1H), 4.06 (d, J=9.9 Hz, 1H), 3.84-3.71 (m, 1H), 3.66 (dd, J=8.8, 4.1 Hz, 1H), 2.87-2.64 (m, 2H), 1.41 (d, J=6.9 Hz, 3H), 1.26 (d, J=6.9 Hz, 6H); ¹³C NMR (75 MHz, CDCl₃) δ 172.4, 142.9, 135.7, 132.2, 128.6, 128.3, 127.3, 125.7, 123.8, 96.7, 79.1, 64.9, 54.0, 49.0, 22.9, 22.0, 21.1; MS(ESI) m/z 336.2 [M+H]⁺; HPLC, >99%, t_{R} 15.8 min.

### Synthesis of Compounds 28 and 29 (Scheme D)

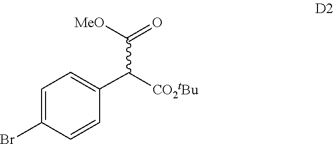 [SMILES: COC(=O)C(C(=O)OC(C)(C)C)c1ccc(Br)cc1]

1-tert-Butyl 3-Methyl 2-(4-bromophenyl)propanedioate (D2). The procedure for the synthesis of A3 was followed starting with 1-bromo-4-iodobenzene (D1) to afford D2 (53% yield) as a colorless oil: ¹H NMR (300 MHz, CDCl₃) δ 7.47 (d, J=8.5 Hz, 2H), 7.27 (d, J=8.5 Hz, 2H), 4.52 (s, 1H), 3.72 (s, 3H), 1.44 (s, 9H); ¹³C NMR (75 MHz, CDCl₃) δ 168.4, 166.6, 132.2, 131.6, 131.0, 127.3, 82.6, 58.1, 52.6, 27.9.

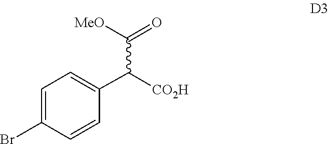 [SMILES: COC(=O)C(C(=O)O)c1ccc(Br)cc1]

2-(4-Bromophenyl)-3-methoxy-3-oxopropanoic Acid (D3). The procedure for the synthesis of A4 was followed starting with D2 to afford crude D3 (quantitative yield), which was used in the next transformation without purification.

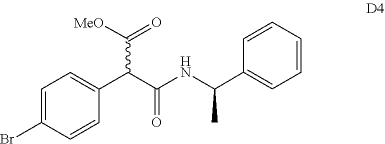 [SMILES: COC(=O)C(C(=O)N[C@H](C)c1ccccc1)c1ccc(Br)cc1]

Methyl 2-(4-Bromophenyl)-2-{[(1 R)-1-phenylethyl]carbamoyl}acetate (D4). The procedure for the synthesis of A5 was followed starting with D3 to afford diastereomeric mixture D4 (70% yield) as a yellow solid: ¹H NMR (300 MHz, CDCl₃) δ 7.46 (dd, J=11.5, 8.5 Hz, 2H), 7.38-7.07 (m, 7H), 5.09 (p, J=6.8 Hz, 1H), 4.46 (d, J=4.5 Hz, 1H), 3.73 (d, J=3.7 Hz, 3H), 1.47 (dd, J=6.9, 3.9 Hz, 3H); ¹³C NMR (75 MHz, CDCl₃) δ 170.9, 165.5, 142.7, 133.1, 132.1, 132.1, 130.0, 128.7, 127.4, 126.0, 126.0, 122.5, 57.5, 52.9, 49.2, 49.1, 21.8, 21.7; MS (ESI) m/z 376.2 [M+H]⁺ (⁷⁹Br), 378.2 [M+H]⁺ (⁸¹Br).

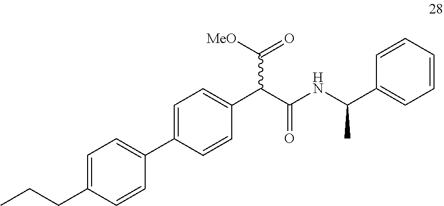 [SMILES: CCCc1ccc(-c2ccc(C(C(=O)N[C@H](C)c3ccccc3)C(=O)OC)cc2)cc1]

Methyl 2-{[(1 R)-1-Phenylethyl]carbamoyl}-2-{4′-propyl-[1,1′-biphenyl]-4-yl}acetate (28). The procedure for the synthesis of 25 was followed starting with D4 to afford diastereomeric mixture 28 (78% yield) as a brown solid: ¹H NMR (300 MHz, CDCl₃) δ 7.66-7.35 (m, 5H), 7.35-7.12 (m, 8H), 5.21-4.96 (m, 1H), 4.61-4.41 (m, 1H), 3.81-3.65 (m, 4H), 2.62 (t, J=7.6 Hz, 2H), 1.76-1.59 (m, 2H), 1.53-1.40 (m, 3H), 0.97 (t, J=7.3 Hz, 3H); MS (ESI) m/z 416.6 [M+H]⁺; HPLC, >99%, t_{R} 18.5 min, 18.6 min (1:1 two peaks).

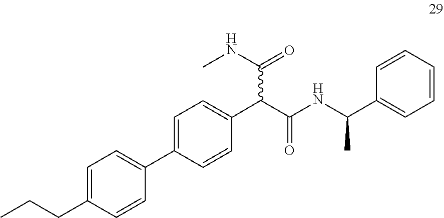 [SMILES: CCCc1ccc(-c2ccc(C(C(=O)NC)C(=O)N[C@H](C)c3ccccc3)cc2)cc1]

N-Methyl-N′-[(1 R)-1-phenylethyl]-2-{4′-propyl-[1,1′-biphenyl]-4-yl}propanediamide (29). To a solution of 28 (100 mg, 0.24 mmol) in MeOH—H₂O (3 mL, 1:1) was added 1 N NaOH (482 μL, 0.48 mmol), and the reaction was stirred at room temperature for 3 h. The mixture was acidified with 1 N HCl to pH=2 and extracted with EtOAc (3×10 mL). The combined organic layers were washed with brine (3×10 mL), dried (Na₂SO₄), and concentrated under reduced pressure to give the corresponding acid (94 mg, 98% yield) as a brownish solid: ¹H NMR (300 MHz, CDCl₃) δ 7.66-6.99 (m, 13H), 6.61 (br s, 1H), 5.11 (dd, J=13.6, 6.9 Hz, 1H), 4.48 (d, J=14.3 Hz, 1H), 2.74-2.53 (m, 2H), 1.82-1.55 (m, 2H), 1.51-1.30 (m, 3H), 0.97 (t, J=7.3 Hz, 3H); MS (ESI) m/z402.4 [M+H]⁺. To a solution of the above acid (60 mg, 0.15 mmol) in MeCN (2 mL) were added TCFH (46 mg, 0.16 mmol), NMI (25 μL, 0.31 mmol), and methylamine hydrochloride (15 mg, 0.22 mmol), and the resulting reaction was stirred at room temperature overnight. The mixture was diluted with EtOAc (10 mL) and H₂O (15 mL). The layers were separated, and the aqueous layer was extracted with additional EtOAc (2×10 mL). The combined organic layers were washed with brine (3×15 mL), dried (Na₂SO₄), and concentrated under reduced pressure. The residue was subjected to chromatography on silica gel using 0-100% EtOAc in hexanes to afford diastereomeric mixture 29 (44 mg, 71% yield) as a white solid: ¹H NMR (300 MHz, CDCl₃) δ 7.59-7.36 (m, 5H), 7.35-7.12 (m, 8H), 5.03 (dd, J=15.6, 8.6 Hz, 1H), 4.62-4.36 (m, 1H), 2.86 (s, 3H), 2.83-2.75 (m, 2H), 2.71-2.54 (m, 2H), 1.76-1.57 (m, 2H), 1.53-1.39 (m, 3H), 0.97 (t, J=7.3 Hz, 3H); MS (ESI) m/z415.4 [M+H]⁺; HPLC, >99%, t_{R} 18.0 min (minor), 18.1 min (major).

### Synthesis of Compounds 38-60 (Scheme E)

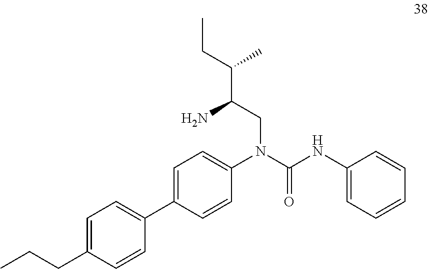 [SMILES: CCCc1ccc(-c2ccc(N(C[C@@H](N)[C@@H](C)CC)C(=O)Nc3ccccc3)cc2)cc1]

3-[(2S,3S)-2-Amino-3-methylpentyl]-1-phenyl-3-{4′-propyl-[1,1′-biphenyl]-4-yl}urea (38). To a solution of tert-butyl N-[(2S,3S)-3-methyl-1-({4′-propyl-[1,1′-biphenyl]-4-yl}amino)pentan-2-yl]carbamate (E1a) (30 mg, 0.07 mmol) in DCM (1 mL) was added phenylisocyante (E2a) (10.5 μL, 0.09 mmol). The reaction was stirred overnight at rt. The mixture was concentrated and the residue was purified by column chromatography on silica gel using EtOAc in hexanes to afford the Boc-protected urea E3a (23.6 mg, 75% yield) as an off-white solid: ¹H NMR (300 MHz, CDCl₃) δ 7.69 (d, J=8.3 Hz, 2H), 7.53 (d, J=8.1 Hz, 2H), 7.43 (d, J=8.1 Hz, 2H), 7.33-7.17 (m, 7H), 7.06-6.93 (m, 1H), 6.22 (s, 1H), 5.24 (d, J=9.1 Hz, 1H), 4.37 (dd, J=14.2, 11.5 Hz, 1H), 3.77 (tt, J=9.1, 4.2 Hz, 1H), 3.22 (dd, J=14.3, 4.0 Hz, 1H), 2.65 (t, J=7.6 Hz, 2H), 1.71 (t, J=7.5 Hz, 2H), 1.60-1.46 (m, 1H), 1.40 (s, 9H), 1.21-1.04 (m, 1H), 0.98 (t, J=7.3 Hz, 3H), 0.94-0.87 (m, 3H), 0.84 (d, J=7.3 Hz, 3H); MS (ESI) m/z 530.20 [M+H]⁺. The Boc-protected intermediate E3a (23.6 mg, 0.05 mmol) was dissolved in DCM (5 mL), followed by addition of HCl (4 M in dioxane, 50 μL, 0.5 mmol). The reaction was stirred at room temperature until completion as judged by TLC analysis. The solvent was evaporated, and the residue was triturated with hexanes to afford 38 hydrochloride salt (20.6 mg, 99% yield) as an off-white solid: ¹H NMR (300 MHz, CD₃OD) δ 7.80 (d, J=6.9 Hz, 1H), 7.59 (d, J=7.6 Hz, 1H), 7.54 (d, J=7.3 Hz, 1H), 7.36 (d, J=7.9 Hz, 1H), 7.31-7.17 (m, 2H), 7.03 (t, J=7.2 Hz, 1H), 4.23 (dd, J=15.1, 8.3 Hz, 1H), 3.78 (d, J=14.9 Hz, 1H), 2.64 (t, J=7.5 Hz, 1H), 1.76 (d, J=10.8 Hz, 1H), 1.68 (q, J=7.4 Hz, 1H), 1.40 (q, J=6.7 Hz, 1H), 1.24 (dt, J=10.3, 5.8 Hz, 1H), 0.97 (t, J=7.4 Hz, 3H), 0.86 (t, J=7.1 Hz, 2H); ¹³C NMR (75 MHz, CD₃OD) δ 158.4, 143.7, 142.5, 141.1, 140.0, 138.4, 130.2, 129.8, 129.6, 129.5, 127.9, 124.7, 122.2, 57.1, 51.1, 38.6, 37.1, 26.4, 25.6, 14.2, 14.0, 11.7; MS (ESI) m/z430.20 [M+H]⁺; HPLC, >99%, t_{R} 16.9 min.

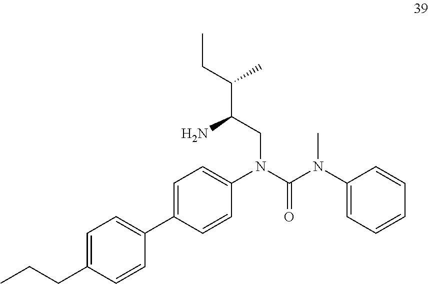 [SMILES: CCCc1ccc(-c2ccc(N(C[C@@H](N)[C@@H](C)CC)C(=O)N(C)c3ccccc3)cc2)cc1]

3-[(2S,3S)-2-Amino-3-methylpentyl]-1-methyl-1-phenyl-3-{4′-propyl-[1,1′-biphenyl]-4-yi}urea (39). To a solution of N-methylaniline (E2b) (10.7 μL, 0.1 mmol) in DCM (1 mL) at 0° C. under nitrogen was added triphosgene (29.6 mg, 0.1 mmol) and the reaction was stirred at room temperature for 2 h. The reaction was quenched with 1 M HCl and extracted with DCM (3×10 mL). The combined organic layers were dried (Na₂SO₄) and concentrated under reduced pressure to afford the corresponding carbamoyl chloride as a colorless residue. To a solution of E1a (45.1 mg, 0.11 mmol), DMAP (2.0 mg), and pyridine (16.1 μL, 0.2 mmol) in DCE (5 mL) at 0° C. under nitrogen, was added the prepared carbamoyl chloride dropwise. The reaction was heated at 90° C. for 12 h. The reaction mixture was cooled to room temperature and diluted with DCM (10 mL). The organic layer was washed with water (2×20 mL), dried (Na₂SO₄), and concentrated under reduced pressure. The residue was subjected to chromatography on silica gel using EtOAc and hexanes to afford the Boc-protected urea E3b (17.5 mg, 32% yield): ¹H NMR (300 MHz, CDCl₃) δ 7.36 (d, J=8.2 Hz, 2H), 7.21 (dd, J=8.3, 6.7 Hz, 4H), 7.01 (t, J=7.5 Hz, 2H), 6.92 (t, J=7.0 Hz, 1H), 6.83-6.69 (m, 4H), 5.64 (d, J=8.0 Hz, 1H), 4.14 (dd, J=14.2, 11.7 Hz, 1H), 3.65 (td, J=8.1, 4.5 Hz, 1H), 3.26 (dd, J=14.2, 3.8 Hz, 1H), 3.16 (s, 3H), 2.67-2.54 (m, 2H), 1.66 (dt, J=15.0, 7.5 Hz, 2H), 1.49 (s, 9H), 1.39 (q, J=7.8 Hz, 2H), 1.09 (dd, J=8.5, 5.9 Hz, 1H), 0.97 (t, J=7.3 Hz, 3H), 0.92-0.76 (m, 6H). Boc group deprotection with 4 M HCl in dioxane following the procedure for the synthesis of 38 afforded 39 hydrochloride salt (9.6 mg, 68% yield) as an off-white solid: ¹H NMR (300 MHz, CD₃OD) δ 7.39 (d, J=7.8 Hz, 2H), 7.31 (d, J=7.6 Hz, 2H), 7.22 (d, J=7.8 Hz, 2H), 7.12-6.88 (m, 5H), 6.84 (d, J=7.5 Hz, 2H), 4.06 (dd, J=15.3, 8.5 Hz, 1H), 3.69 (d, J=15.3 Hz, 1H), 3.23 (s, 3H), 3.13 (d, J=7.6 Hz, 1H), 2.61 (t, J=7.5 Hz, 2H), 1.66 (h, J=7.3 Hz, 3H), 1.33-1.06 (m, 2H), 1.01-0.85 (m, 6H), 0.74 (t, J=7.2 Hz, 3H); ¹³C NMR (75 MHz, CD₃OD) δ 163.7, 145.8, 143.3, 142.1, 140.2, 138.7, 130.0, 128.5, 128.2, 127.6, 127.4, 126.9, 56.6, 52.1, 40.1, 38.6, 37.3, 26.4, 25.7, 14.1, 14.0, 11.5; MS (ESI) m/z444.20 [M+H]⁺; HPLC, >98%, t_{R} 17.6 min.

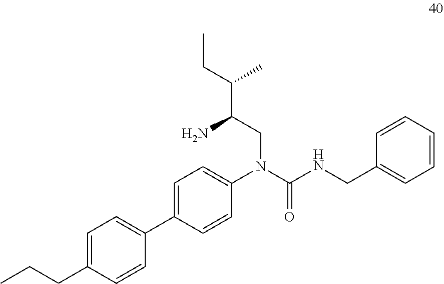 [SMILES: CCCc1ccc(-c2ccc(N(C[C@@H](N)[C@@H](C)CC)C(=O)NCc3ccccc3)cc2)cc1]

3-[(2S,3S)-2-Amino-3-methylpentyl]-1-benzyl-3-{4′-propyl-[1,1′-biphenyl]-4-yl}urea (40). The procedure for the synthesis of 38 was followed starting with E1a and benzylisocyanate (E2c) to afford the Boc-protected urea E3c (80% yield), which was then deprotected with HCl to give 40 hydrochloride salt (95% yield) as an off-white solid: ¹H NMR (300 MHz, CD₃OD) δ 7.75 (d, J=8.4 Hz, 2H), 7.56 (d, J=8.0 Hz, 2H), 7.45 (d, J=8.3 Hz, 2H), 7.32-6.95 (m, 7H), 4.83-4.63 (m, 1H), 4.38-4.18 (m, 2H), 4.12 (dd, J=15.4, 8.8 Hz, 1H), 3.87-3.54 (m, 1H), 3.27-3.17 (m, 1H), 3.28-3.14 (m, 1H), 2.63 (t, J=7.5 Hz, 2H), 1.83-1.50 (m, 3H), 1.50-1.29 (m, 1H), 1.29-1.07 (m, 1H), 0.96 (t, J=7.9 Hz, 6H), 0.86 (t, J=7.4 Hz, 3H); ¹³C NMR (75 MHz, CD₃OD) δ 160.8, 149.7, 142.6, 141.4, 141.1, 138.5, 130.2, 129.8, 129.7, 129.4, 128.2, 128.0, 127.9, 57.4, 51.3, 45.4, 38.6, 37.0, 26.2, 25.7, 14.3, 14.0, 11.6; MS (ESI) m/z 444.40 [M+H]⁺; HPLC, >99%, t_{R} 17.0 min.

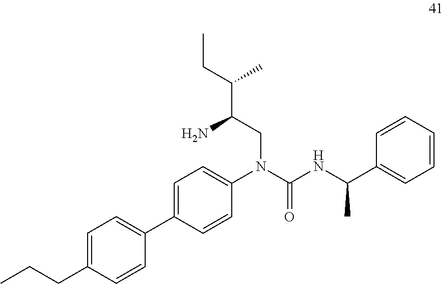 [SMILES: CCCc1ccc(-c2ccc(N(C[C@@H](N)[C@@H](C)CC)C(=O)N[C@H](C)c3ccccc3)cc2)cc1]

3-[(2S,3S)-2-Amino-3-methylpentyl]-1-[(1 R)-1-phenylethyl]-3-{4′-propyl-[1,1′-biphenyl]-4-yl}urea (41). The procedure for the synthesis of 38 was followed starting with Ela and (R)-(+)-α-methylbenzyl isocyanate (E2d) to afford the Boc-protected urea E3d (65% yield), which was then deprotected with HCl to give 41 hydrochloride salt (76% yield) as a white solid: ¹H NMR (400 MHz, CD₃OD) δ 7.73 (d, J=8.4 Hz, 2H), 7.55 (d, J=8.1 Hz, 2H), 7.40 (d, J=8.4 Hz, 2H), 7.32-7.12 (m, 7H), 4.95 (q, J=7.0 Hz, 1H), 4.05 (dd, J=15.3, 8.7 Hz, 1H), 3.72 (dd, J=15.3, 2.6 Hz, 1H), 3.23-3.08 (m, 1H), 2.62 (t, J=7.6 Hz, 2H), 1.80-1.51 (m, 3H), 1.46-1.26 (m, 4H), 1.26-1.06 (m, 1H), 0.94 (t, J=7.3 Hz, 3H), 0.90 (d, J=6.9 Hz, 3H), 0.82 (t, J=7.4 Hz, 3H); ¹³C NMR (100 MHz, CD₃OD) δ 158.6, 144.4, 142.4, 141.1, 140.1, 137.1, 128.8, 128.4, 128.2, 126.7, 126.5, 125.7, 55.9, 50.6, 49.9, 37.3, 35.7, 24.9, 24.4, 21.4, 12.9, 12.7, 10.3; MS (ESI) m/z458.30 [M+H]⁺; HPLC, >99%, t_{R} 17.5 min.

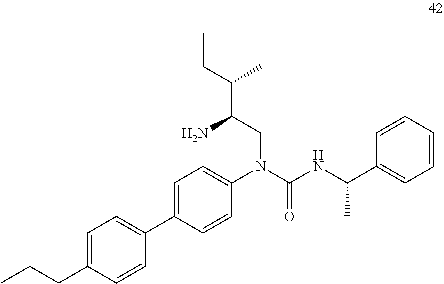 [SMILES: CCCc1ccc(-c2ccc(N(C[C@@H](N)[C@@H](C)CC)C(=O)N[C@@H](C)c3ccccc3)cc2)cc1]

3-[(2S,3S)-2-Amino-3-methylpentyl]-1-[(1 S)-1-phenylethyl]-3-{4′-propyl-[1,1′-biphenyl]-4-yl}urea (42). The procedure for the synthesis of 38 was followed starting with Ela and (S)-(−)-α-methylbenzyl isocyanate (E2e) to afford the Boc-protected urea E3e (76% yield), which as then deprotected with HCl to give 42 hydrochloride salt (80% yield) as a white solid: ¹H NMR (400 MHz, CD₃OD) δ 7.75 (d, J=8.5 Hz, 2H), 7.56 (d, J=8.1 Hz, 2H), 7.43 (d, J=8.5 Hz, 2H), 7.30-7.11 (m, 7H), 4.97-4.89 (m, 1H), 4.05 (dd, J=15.3, 8.8 Hz, 1H), 3.78-3.53 (m, 1H), 3.23-3.14 (m, 1H), 2.62 (t, J=7.6 Hz, 2H), 1.78-1.50 (m, 3H), 1.40-1.29 (m, 4H), 1.21-1.08 (m, 1H), 1.00-0.85 (m, 6H), 0.80 (t, J=7.4 Hz, 3H); MS (ESI) m/z458.30 [M+H]⁺; HPLC, >99%, t_{R} 17.7 min.

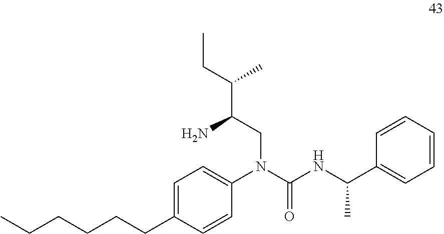 [SMILES: CCCCCCc1ccc(N(C[C@@H](N)[C@@H](C)CC)C(=O)N[C@@H](C)c2ccccc2)cc1]

3-[(2S,3S)-2-Amino-3-methylpentyl]-3-(4-hexylphenyl)-1-[(1 S)-1-phenylethyl]urea (43). The procedure for the synthesis of 38 was followed starting with tert-butyl N-[(2S,3S)-1-[(4-hexylphenyl)amino]-3-methylpentan-2-yl]carbamate (E1b) and (S)-(−)-α-methylbenzyl isocyanate (E2e) to afford the Boc-protected urea E3f (63% yield), which was then deprotected with HCl to give 43 hydrochloride salt (quantitative yield) as a white solid: ¹H NMR (300 MHz, CD₃OD) δ 6.97-7.37 (m, 9H), 3.96 (dd, J=15.26, 8.85 Hz, 1H), 3.50-3.69 (m, 2H), 3.49-3.56 (m, 1H), 3.41-3.50 (m, 1H), 3.04-3.15 (m, 1H), 2.57 (t, J=7.63 Hz, 2H), 1.45-1.68 (m, 3H), 1.15-1.32 (m, 10H), 1.07 (dd, J=15.26, 6.97 Hz, 1H), 0.74-0.87 (m, 4H), 0.69 (t, J=7.35 Hz, 3H); ¹³C NMR (75 MHz, CD₃OD) δ 159.8, 145.9, 144.8, 140.1, 131.6, 129.5, 129.2, 127.9, 126.8, 57.1, 51.8, 51.0, 36.9, 36.4, 32.8, 32.4, 30.0, 26.3, 23.7, 22.7, 14.4, 14.3, 11.6; MS (ESI) m/z 424.30 [M+H]⁺; HPLC, >98%, t_{R} 17.7 min.

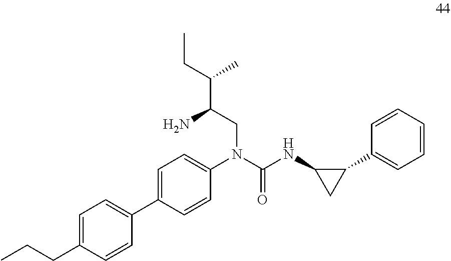 [SMILES: CCCc1ccc(-c2ccc(N(C[C@@H](N)[C@@H](C)CC)C(=O)N[C@@H]3C[C@H]3c3ccccc3)cc2)cc1]

1-[(2S,3S)-2-Amino-3-methylpentyl]-3-[(1 R,2S)-2-phenylcyclopropyl]-1-(4′-propyl-[1,1′-biphenyl]-4-yl}urea (44). The procedure for the synthesis of 38 was followed starting with E1a and [(1 S,2R)-2-isocyanatocyclopropyl]benzene (E2f) to afford the Boc-protected urea E3g (50% yield), which was then deprotected with HCl to give 44 hydrochloride salt (60% yield) as a white solid: ¹H NMR (400 MHz, CD₃OD) δ 7.72 (d, J=8.5 Hz, 2H), 7.54 (d, J=8.2 Hz, 2H), 7.39 (d, J=8.5 Hz, 2H), 7.26 (d, J=8.2 Hz, 2H), 7.20 (t, J=7.4 Hz, 2H), 7.14-7.04 (m, 3H), 4.11 (dd, J=15.3, 9.0 Hz, 1H), 3.68 (dd, J=15.3, 2.7 Hz, 1H), 3.26-3.17 (m, 1H), 2.82-2.68 (m, 1H), 2.68-2.54 (m, 2H), 2.02-1.87 (m, 1H), 1.79-1.54 (m, 3H), 1.44-1.30 (m, 1H), 1.25-1.11 (m, 1H), 1.11-1.03 (m, 2H), 1.03-0.87 (m, 6H), 0.83 (t, J=7.4 Hz, 3H); ¹³C NMR (100 MHz, CD₃OD) δ 160.1, 142.4, 141.3, 141.0, 139.9, 137.2, 128.8, 128.4, 128.1, 127.9, 126.5, 125.8, 125.5, 55.8, 49.7, 37.3, 35.7, 33.3, 25.0, 24.4, 24.0, 15.3, 12.9, 12.7, 10.3; MS (ESI) m/z 470.40 [M+H]⁺; HPLC, >97%, t_{R} 17.6 min.

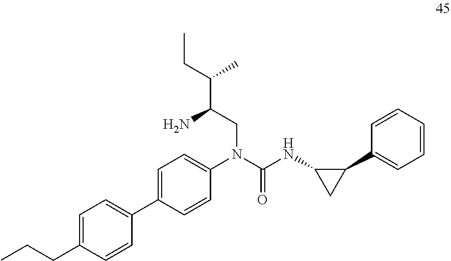 [SMILES: CCCc1ccc(-c2ccc(N(C[C@@H](N)[C@@H](C)CC)C(=O)N[C@H]3C[C@@H]3c3ccccc3)cc2)cc1]

1-[(2S,3S)-2-Amino-3-methylpentyl]-3-[(1 S,2R)-2-phenylcyclopropyl]-1-(4′-propyl-[1,1′-biphenyl]-4-yl}urea (45). The procedure for the synthesis of 38 was followed starting with E1a and [(1 R,2S)-2-isocyanatocyclopropyl]benzene (E2g) to afford the Boc-protected urea E3h (60% yield), which was then deprotected with HCl to give 45 hydrochloride salt (95% yield) as a white solid: ¹H NMR (400 MHz, CD₃OD) δ 7.72 (d, J=7.5 Hz, 2H), 7.55 (d, J=7.3 Hz, 2H), 7.39 (d, J=7.5 Hz, 2H), 7.27 (d, J=7.5 Hz, 2H), 7.20 (t, J=7.6 Hz, 2H), 7.14-7.03 (m, 3H), 4.12 (dd, J=15.2, 9.0 Hz, 1H), 3.69 (d, J=1.5 Hz, 1H), 3.26-3.16 (m, 1H), 2.79-2.67 (m, 1H), 2.62 (t, J=7.5 Hz, 2H), 1.98-1.86 (m, 1H), 1.76-1.57 (m, 3H), 1.45-1.31 (m, 1H), 1.25-1.14 (m, 1H), 1.14-1.04 (m, 2H), 1.02-0.88 (m, 6H), 0.83 (t, J=7.2 Hz, 3H); ¹³C NMR (75 MHz, CD₃OD) δ 160.1, 142.4, 141.3, 141.0, 140.0, 137.2, 128.8, 128.4, 128.2, 127.9, 126.5, 125.8, 125.5, 55.8, 49.7, 39.6, 37.3, 33.3, 25.0, 24.4, 24.3, 15.0, 12.9, 12.7, 10.3, MS (ESI) m/z 470.40 [M+H]⁺; HPLC, >95%, t_{R} 17.6 min.

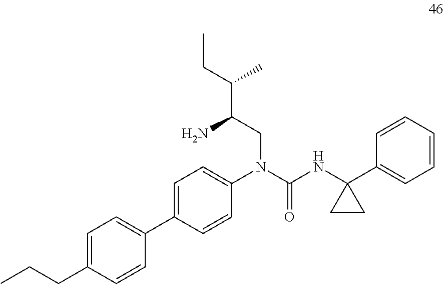 [SMILES: CCCc1ccc(-c2ccc(N(C[C@@H](N)[C@@H](C)CC)C(=O)NC3(c4ccccc4)CC3)cc2)cc1]

1-[(2S,3S)-2-Amino-3-methylpentyl]-3-(1-phenylcyclopropyl)-1-{4′-propyl-[1,1′-biphenyl]-4-yl}urea (46). Triphosgene (27 mg, 0.09 mmol) was dissolved in DCM (1 mL) at room temperature. A mixture of 1-phenylcyclopropan-1-amine (E2h) (33 mg, 0.25 mmol) and DIPEA (52 μL, 0.3 mmol) in DCM (1 mL) was added to the above solution dropwise over a period of 5 min. After stirring for 20 min, a solution of E1a (102 mg, 0.25 mmol) and DIPEA (52 μL, 0.3 mmol) in DCM (1 mL) was added in one portion. The reaction was stirred for at room temperature for 2 d. The mixture was concentrated under reduced pressure and the residue was subjected to chromatography on silica gel using EtOAc in hexanes to afford the Boc-protected urea E3i (29% yield) as a clear residue: MS (ESI) m/z 570.4 [M+H]⁺. Boc group deprotection with TFA in DCM afforded 46 trifluoroacetate salt (32 mg, 76% yield) as a white solid: ¹H NMR (300 MHz, CD₃OD) δ 7.76 (d, J=8.4 Hz, 2H), 7.57 (d, J=8.2 Hz, 2H), 7.44 (d, J=8.5 Hz, 2H), 7.32-7.17 (m, 6H), 7.17-7.06 (m, 1H), 4.06 (dd, J=15.2, 8.7 Hz, 1H), 3.74 (dd, J=15.2, 3.1 Hz, 1H), 3.28-3.17 (m, 1H), 2.63 (t, J=7.5 Hz, 2H), 1.85-1.57 (m, 3H), 1.52-1.32 (m, 1H), 1.31-1.05 (m, 5H), 1.03-0.90 (m, 6H), 0.84 (t, J=7.3 Hz, 3H); ¹³C NMR (75 MHz, CD₃OD) δ 160.9, 144.9, 143.7, 142.5, 141.5, 138.6, 130.2, 129.9, 129.6, 129.2, 127.9, 127.0, 126.2, 57.3, 51.3, 38.7, 37.0, 36.4, 26.2, 25.7, 19.1, 18.9, 14.3, 14.1, 11.7; MS (ESI) m/z 470.4 [M+H]⁺; HPLC, >99%, t_{R} 17.3 min.

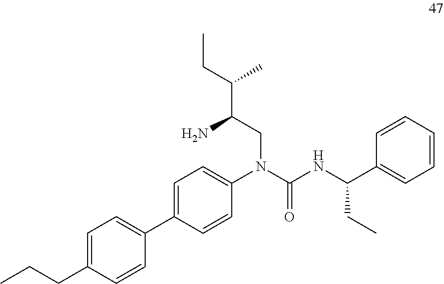 [SMILES: CCCc1ccc(-c2ccc(N(C[C@@H](N)[C@@H](C)CC)C(=O)N[C@@H](CC)c3ccccc3)cc2)cc1]

3-[(2S,3S)-2-Amino-3-methylpentyl]-1-[(1 S)-1-phenylpropyl]-3-{4′-propyl-[1,1′-biphenyl]-4-yl}urea (47). The procedure for the synthesis of 46 was followed starting with E1a and (1 S)-1-phenylpropan-1-amine (E2i) to afford the Boc-protected urea E3j (46% yield), which was then deprotected with HCl to give 47 hydrochloride salt (90% yield) as an off-white solid:¹H NMR (300 MHz, CD₃OD) δ 7.78 (d, J=8.2 Hz, 2H), 7.59 (d, J=7.9 Hz, 2H), 7.46 (d, J=8.1 Hz, 2H), 7.34-7.24 (m, 4H), 7.24-7.13 (m, 3H), 4.64 (t, J=7.4 Hz, 1H), 4.03 (dd, J=15.3, 8.6 Hz, 1H), 3.71 (dd, J=15.5, 2.8 Hz, 1H), 3.27-3.14 (m, 1H), 2.64 (t, J=7.5 Hz, 2H), 1.75-1.59 (m, 5H), 1.46-1.06 (m, 2H), 0.97 (t, J=7.3 Hz, 3H), 0.93-0.75 (m, 9H); ¹³C NMR (75 MHz, CD₃OD) δ 160.1, 144.9, 143.8, 142.5, 141.5, 138.5, 130.2, 129.9, 129.7, 129.4, 128.0, 127.9, 127.5, 58.5, 57.4, 51.3, 38.7, 37.0, 30.5, 26.3, 25.7, 14.4, 14.1, 11.7, 11.6; MS (ESI) m/z472.4 [M+H]⁺; HPLC, >99%, t_{R} 18.1 min.

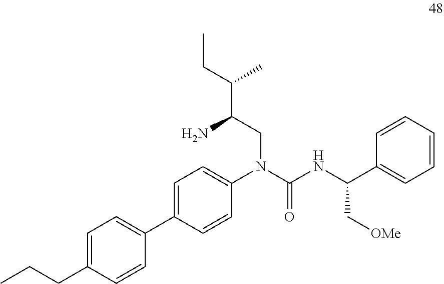 [SMILES: CCCc1ccc(-c2ccc(N(C[C@@H](N)[C@@H](C)CC)C(=O)N[C@@H](COC)c3ccccc3)cc2)cc1]

3-[(2S,3S)-2-Amino-3-methylpentyl]-1-[(1 R)-2-methoxy-1-phenylethyl]-3-{4′-propyl-[1,1′-biphenyl]-4-yl}urea (48). The procedure for the synthesis of 46 was followed starting with E1a and (1 S)-1-phenylpropan-1-amine (E2j) to afford the Boc-protected urea E3k (57% yield), which was then deprotected with HCl to give 48 hydrochloride salt (quantitative yield) as an off-white solid: ¹H NMR (300 MHz, CD₃OD) δ 7.80 (d, J=7.6 Hz, 2H), 7.59 (d, J=7.7 Hz, 2H), 7.50 (d, J=7.8 Hz, 2H), 7.36-7.19 (m, 7H), 5.02 (t, J=6.1 Hz, 1H), 4.16 (dd, J=15.2, 8.9 Hz, 1H), 3.64 (d, J=15.3 Hz, 1H), 3.58-3.44 (m, 2H), 3.30 (s, 3H), 3.26-3.15 (m, 1H), 2.64 (t, J=7.5 Hz, 2H), 1.69 (h, J=7.4 Hz, 3H), 1.42-1.30 (m, 1H), 1.26-1.10 (m, 1H), 1.03-0.92 (m, 6H), 0.83 (t, J=7.2 Hz, 3H); ¹³C NMR (75 MHz, CD₃OD) δ 160.0, 143.8, 142.7, 141.7, 141.2, 138.4, 130.2, 129.9, 129.7, 129.4, 128.4, 127.9, 127.7, 76.5, 59.3, 57.3, 56.1, 50.9, 38.7, 37.1, 26.4, 25.7, 14.2, 14.1, 11.7; MS (ESI) m/z 488.4 [M+H]⁺; HPLC, >99%, t_{R} 17.4 min.

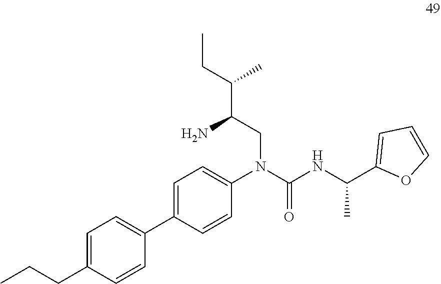 [SMILES: CCCc1ccc(-c2ccc(N(C[C@@H](N)[C@@H](C)CC)C(=O)N[C@@H](C)c3ccco3)cc2)cc1]

1-[(2S,3S)-2-Amino-3-methylpentyl]-3-[(1 S)-1-(furan-2-yl)ethyl]-1-{4′-propyl-[1,1′-biphenyl]-4-yl}urea (49). The procedure for the synthesis of 46 was followed staring with E1a and (1 S)-1-(furan-2-yl)ethan-1-amine (E2k) to afford the Boc-protected urea E3l (20% yield), which was then deprotected with HCl to give 49 hydrochloride salt (55% yield) as an off-white solid: MS (ESI) m/z 448.40 [M+H]⁺; HPLC, >95%, t_{R} 17.1 min.

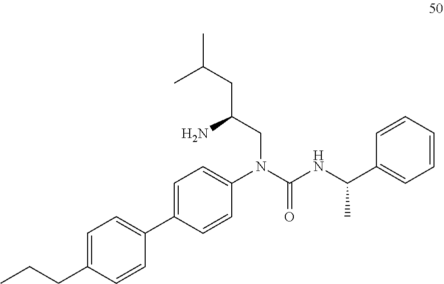 [SMILES: CCCc1ccc(-c2ccc(N(C[C@@H](N)CC(C)C)C(=O)N[C@@H](C)c3ccccc3)cc2)cc1]

3-[(2S)-2-Amino-4-methylpentyl]-1-[(1 S)-1-phenylethyl]-3-{4′-propyl-[1,1′-biphenyl]-4-yl}urea (50). The procedure for the synthesis of 38 was followed starting with tert-butyl N-[(2S)-4-methyl-1-({4′-propyl-[1,1′-biphenyl]-4-yl}amino)pentan-2-yl]carbamate (E1c) and (S)-(−)-α-methylbenzyl isocyanate (E2e) to afford the Boc-protected urea E3m (60% yield), which was then deprotected with HCl to give 50 hydrochloride salt (90% yield) as a white solid: ¹H NMR (400 MHz, CD₃OD) δ 7.74 (d, J=8.3 Hz, 2H), 7.55 (d, J=8.1 Hz, 2H), 7.43 (d, J=8.3 Hz, 2H), 7.31-7.10 (m, 7), 4.98-4.88 (m, 1H), 3.95 (dd, J=15.2, 7.8 Hz, 1H), 3.76 (dd, J=15.2, 3.0 Hz, 1H), 3.36-3.31 (m, 1H), 2.61 (t, J=7.5 Hz, 2H), 1.74-1.58 (m, 2H), 1.58-1.42 (m, 2H), 1.42-1.24 (m, 4H), 0.94 (t, J=7.3 Hz, 3H), 0.88-0.70 (m, 6H); ¹³C NMR (75 MHz, CD₃OD) δ 158.4 150.3, 144.6, 142.4, 141.1, 140.4, 137.1, 128.8, 128.4, 128.1, 126.6, 126.5, 125.6, 52.1, 50.6, 50.3, 39.3, 37.3, 24.4, 24.1, 21.3, 20.9, 12.7; MS (ESI) m/z458.4 [M+H]⁺; HPLC, >99%, t_{R} 17.5 min.

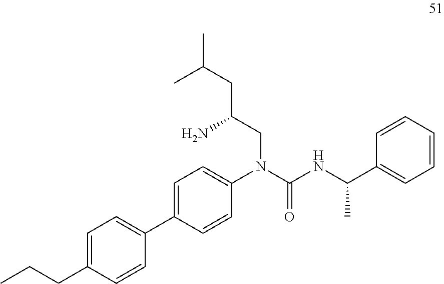 [SMILES: CCCc1ccc(-c2ccc(N(C[C@H](N)CC(C)C)C(=O)N[C@@H](C)c3ccccc3)cc2)cc1]

3-[(2R)-2-Amino-4-methylpentyl]-1-[(1 S)-1-phenylethyl]-3-{4′-propyl-[1,1′-biphenyl]-4-yl}urea (51). The procedure for the synthesis of 38 was followed starting with tert-butyl N-[(2R)-4-methyl-1-({4′-propyl-[1,1′-biphenyl]-4-yl}amino)pentan-2-yl]carbamate (E1d) and (S)-(−)-α-methylbenzyl isocyanate (E2e) to afford the Boc-protected urea E3n (70% yield), which was then deprotected with HCl to give 51 hydrochloride salt (90% yield) as a white solid: ¹H NMR (400 MHz, CD₃OD) δ 7.73 (d, J=8.3 Hz, 2H), 7.54 (d, J=8.1 Hz, 2H), 7.41 (d, J=8.3 Hz, 2H), 7.32-7.12 (m, 7H), 4.93 (q, J=7.0 Hz, 1H), 3.93 (dd, J=15.2, 7.4 Hz, 1H), 3.80 (dd, J=15.1, 3.0 Hz, 1H), 3.38-3.30 (m, 1H), 2.61 (t, J=7.5 Hz, 2H), 1.77-1.57 (m, 2H), 1.57-1.44 (m, 2H), 1.44-1.23 (m, 4H), 0.94 (t, J=7.3 Hz, 3H), 0.80 (d, J=6.0 Hz, 6H); ¹³C NMR (75 MHz, CD₃OD) δ 158.5, 144.4, 142.4, 141.0, 140.5, 137.1, 128.8, 128.4, 128.1, 128.1, 126.6, 126.5, 125.7, 52.2, 50.6, 50.3, 39.3, 37.3, 24.4, 24.1, 21.4, 21.3, 20.9, 12.7; MS (ESI) m/z458.35 [M+H]⁺; HPLC, >99%, t_{R} 17.6 min

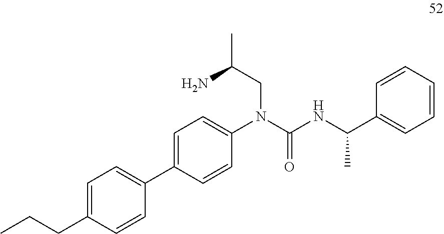 [SMILES: CCCc1ccc(-c2ccc(N(C[C@H](C)N)C(=O)N[C@@H](C)c3ccccc3)cc2)cc1]

3-[(2S)-2-Aminopropyl]-1-[(1 S)-1-phenylethyl]-3-{4′-propyl-[1,1′-biphenyl]-4-yl}urea (52). The procedure for the synthesis of 38 was followed starting with tert-butyl N-[(2S)-1-({4′-propyl-[1,1′-biphenyl]-4-yl}amino)propan-2-yl]carbamate (E1e) and (S)-(−)-α-methylbenzyl isocyanate (E2e) to afford the Boc-protected E3o (65% yield), which was then deprotected with HCl to give 52 hydrochloride salt (70% yield) as a white solid: ¹H NMR (400 MHz, CD₃OD) δ 7.74 (d, J=8.3 Hz, 2H), 7.54 (d, J=8.1 Hz, 2H), 7.42 (d, J=8.3 Hz, 2H), 7.31-7.08 (m, 7H), 4.98-4.88 (m, 1H), 3.97 (dd, J=14.9, 8.1 Hz, 1H), 3.67 (dd, J=14.9, 4.1 Hz, 1H), 3.47-3.32 (m, 1H), 2.67-2.44 (m, 2H), 1.78-1.54 (m, 2H), 1.31 (t, J=9.3 Hz, 3H), 1.23 (t, J=10.4 Hz, 3H), 0.94 (t, J=7.3 Hz, 3H); ¹³C NMR (100 MHz, CD₃OD) δ 158.1, 144.6, 142.4, 141.1, 140.3, 137.2, 128.8, 128.5, 128.3, 128.1, 126.6, 126.5, 125.5, 53.4, 50.5, 42.6, 37.3, 24.4, 21.4, 15.3, 12.7; MS (ESI) m/z416.20 [M+H]⁺; HPLC, >97%, t_{R} 16.2 min.

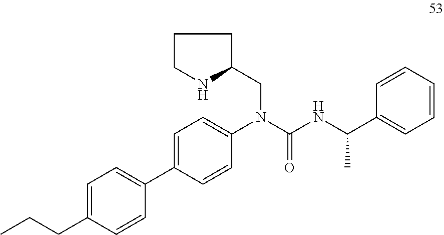 [SMILES: CCCc1ccc(-c2ccc(N(C[C@@H]3CCCN3)C(=O)N[C@@H](C)c3ccccc3)cc2)cc1]

1-[(1 S)-1-Phenylethyl]-3-{4′-propyl-[1,1′-biphenyl]-4-yl}-3-{[(2S)-pyrrolidin-2-yl]methyl}urea (53). The procedure for the synthesis of 38 was followed starting with tert-butyl (2S)-2-[({4′-propyl-[1,1′-biphenyl]-4-yl}amino)methyl]pyrrolidine-1-carboxylate (Elf) and (S)-(−)-α-methylbenzyl isocyanate (E2e) to afford the Boc-protected urea E3p (99% yield), which was then deprotected with HCl to give 53 hydrochloride salt (98% yield) as a white solid: ¹H NMR (300 MHz, CD₃OD) δ 7.77 (d, J=7.0 Hz, 2H), 7.57 (d, J=7.6 Hz, 2H), 7.46 (d, J=7.2 Hz, 2H), 7.35-7.10 (m, 8H), 4.94 (d, J=6.8 Hz, 1H), 4.26-3.96 (m, 1H), 3.78 (d, J=12.6 Hz, 2H), 2.74-2.47 (m, 2H), 2.19-1.82 (m, 3H), 1.68 (q, J=7.4 Hz, 3H), 1.36 (d, J=6.6 Hz, 3H), 0.96 (t, J=7.2 Hz, 3H), 0.88 (q, J=7.1, 6.7 Hz, 1H); ¹³C NMR (75 MHz, CD₃OD) δ 160.0, 145.9, 143.7, 142.4, 141.5, 138.5, 130.1, 129.8, 129.6, 129.4, 127.9, 127.7, 126.9, 61.7, 52.9, 52.0, 46.6, 38.6, 28.5, 25.6, 24.7, 22.5, 14.0; MS (ESI) m/z 442.40 [M+H]⁺; HPLC, >99%, t_{R} 16.9 min.

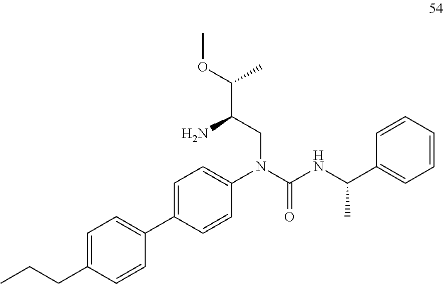 [SMILES: CCCc1ccc(-c2ccc(N(C[C@@H](N)[C@@H](C)OC)C(=O)N[C@@H](C)c3ccccc3)cc2)cc1]

3-[(2R,3R)-2-Amino-3-methoxybutyl]-1-[(1 S)-1-phenylethyl]-3-{4′-propyl-[1,1′-biphenyl]-4-yl}urea (54). The procedure for the synthesis of 38 was followed starting with tert-butyl N-[(2R,3R)-3-methoxy-1-({4′-propyl-[1,1′-biphenyl]-4-yl}amino)butan-2-yl]carbamate (E1g) and (S)-(−)-α-methylbenzyl isocyanate (E2e) to afford the Boc-protected urea E3q (70% yield), which was then deprotected with HCl to give 54 hydrochloride salt (83% yield) as a white solid: ¹H NMR (300 MHz, CD₃OD) δ 7.77 (d, J=7.9 Hz, 2H), 7.58 (d, J=7.8 Hz, 2H), 7.45 (d, J=8.0 Hz, 2H), 7.35-7.13 (m, 7H), 4.94 (q, J=6.8 Hz, 1H), 4.06 (dd, J=15.1, 7.3 Hz, 1H), 3.83 (dd, J=15.1, 3.9 Hz, 1H), 3.45 (t, J=6.1 Hz, 1H), 3.29 (s, 3H), 3.20 (s, 1H), 2.64 (t, J=7.5 Hz, 2H), 1.68 (h, J=7.3 Hz, 2H), 1.36 (d, J=7.0 Hz, 3H), 1.11 (d, J=6.1 Hz, 3H), 0.97 (t, J=7.3 Hz, 3H); ¹³C NMR (75 MHz, CD₃OD) δ 159.7, 145.9, 143.8, 142.5, 141.6, 138.4, 130.2, 129.8, 129.5, 129.4, 127.9, 127.9, 126.9, 75.0, 57.9, 56.8, 52.0, 51.4, 38.6, 25.7, 22.6, 15.6, 14.0; MS (ESI) m/z460.25 [M+H]⁺; HPLC, >99%, t_{R} 17.0 min.

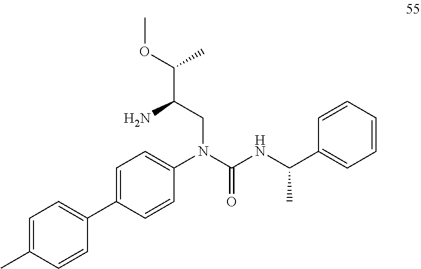 [SMILES: CO[C@H](C)[C@H](N)CN(C(=O)N[C@@H](C)c1ccccc1)c1ccc(-c2ccc(C)cc2)cc1]

3-[(2R,3R)-2-Amino-3-methoxybutyl]-1-[(1 S)-1-phenylethyl]-3-{4′-methyl-[1,1′-biphenyl]-4-yl}urea (55). The procedure for the synthesis of 38 was followed starting with tert-butyl N-[(2R,3R)-1-[(4-bromophenyl)amino]-3-methoxybutan-2-yl]carbamate (E1h) and (S)-(−)-α-methylbenzyl isocyanate (E2e) to afford tert-butyl N-[(2R,3R)-1-[(4-bromophenyl)({[(1S)-1-phenylethyl]carbamoyl})amino]-3-methoxybutan-2-yl]carbamate (E3r) (52% yield) as an off-white solid: ¹H NMR (300 MHz, CDCl₃) δ 7.53 (d, J=8.5 Hz, 2H), 7.39-7.00 (m, 7H), 5.13 (d, J=9.0 Hz, 1H), 4.98 (p, J=7.1 Hz, 1H), 4.74 (d, J=7.9 Hz, 1H), 4.29-3.96 (m, 1H), 3.71 (d, J=4.8 Hz, 1H), 3.52-3.24 (m, 2H), 3.19 (s, 3H), 1.45 (s, 9H), 1.38 (t, J=7.3 Hz, 3H), 1.09 (d, J=6.3 Hz, 3H); MS (ESI) m/z 520.20 [M+H]⁺ (⁷⁹Br), 522.20 [M+H]⁺ (¹¹Br). Suzuki coupling of E3r with 4-methylphenyl boronic acid was carried out following the procedure used for the synthesis of 25 to afford the Boc-protected biphenyl urea E4a (60% yield), which was then deprotected with HCl to give 55 hydrochloride salt (80% yield) as a white solid (80%): ¹H NMR (400 MHz, CD₃OD) δ 7.74 (d, J=8.5 Hz, 2H), 7.53 (d, J=8.1 Hz, 2H), 7.42 (d, J=8.4 Hz, 2H), 7.34-7.09 (m, 7H), 4.95-4.88 (m, 1H), 3.80 (dd, J=15.2, 4.1 Hz, 1H), 3.75-3.52 (m, 1H), 3.42 (p, J=6.2 Hz, 1H), 3.27-3.23 (m, 3H), 3.22-3.11 (m, 1H), 2.36 (s, 3H), 1.33 (d, J=7.1 Hz, 3H), 1.08 (d, J=6.2 Hz, 3H); ¹³C NMR (100 MHz, CD₃OD) δ 158.3, 144.6, 141.1, 140.2, 137.6, 136.8, 129.4, 128.4, 128.1, 128.1, 126.6, 126.5, 125.6, 73.6, 56.4, 55.4, 50.6, 50.0, 21.3, 19.8, 14.2; MS (ESI) m/z432.20 [M+H]⁺; HPLC, >99%, t_{R} 15.3 min.

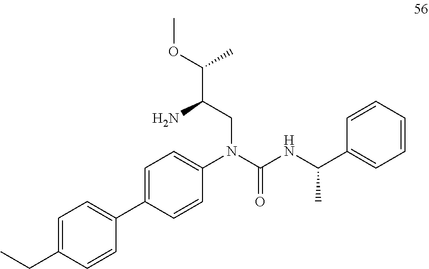 [SMILES: CCc1ccc(-c2ccc(N(C[C@@H](N)[C@@H](C)OC)C(=O)N[C@@H](C)c3ccccc3)cc2)cc1]

3-[(2R,3R)-2-Amino-3-methoxybutyl]-1-[(1 S)-1-phenylethyl]-3-{4′-ethyl-[1,1′-biphenyl]-4-yl}urea (56). The procedure for the synthesis of 55 was followed starting with E3r and 4-ethylphenylboronic acid to afford the Boc-protected biphenyl urea E4b (62% yield), which was then deprotected with HCl to give 56 hydrochloride salt (80% yield) as a white solid: ¹H NMR (300 MHz, CD₃OD) δ 7.78 (d, J=6.7 Hz, 2H), 7.59 (d, J=7.7 Hz, 2H), 7.46 (d, J=7.2 Hz, 2H), 7.36-7.10 (m, 9H), 4.94 (d, J=6.2 Hz, 1H), 4.83-4.69 (m, OH), 3.47 (d, J=8.3 Hz, 1H), 3.29 (s, 3H), 3.21 (s, 3H), 2.69 (q, J=7.5 Hz, 2H), 1.36 (d, J=6.7 Hz, 3H), 1.26 (t, J=7.5 Hz, 3H), 1.11 (d, J=5.3 Hz, 3H); ¹³C NMR (75 MHz, CD₃OD) δ 159.7, 145.9, 145.4, 142.5, 141.63, 138.4, 129.9, 129.6, 129.5, 128.0, 128.0, 127.0, 75.0, 57.9, 56.9, 52.0, 51.5, 29.5, 22.7, 16.1, 15.7; MS (ESI) m/z446.20 [M+H]⁺; HPLC, >99%, t_{R} 14.4 min.

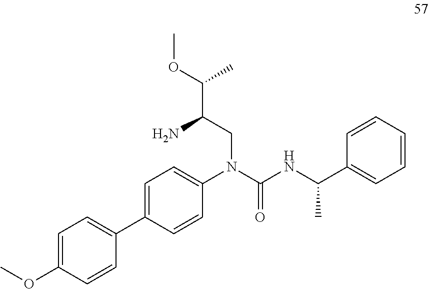 [SMILES: COc1ccc(-c2ccc(N(C[C@@H](N)[C@@H](C)OC)C(=O)N[C@@H](C)c3ccccc3)cc2)cc1]

3-[(2R,3R)-2-Amino-3-methoxybutyl]-1-[(1 S)-1-phenylethyl]-3-{4′-methoxy-[1,1′-biphenyl]-4-yl}urea (57). The procedure for the synthesis of 55 was followed starting with E3r and 4-methoxyphenylboronic acid to afford the Boc-protected biphenyl urea E4c (65% yield), which was then deprotected with HCl to give 57 hydrochloride salt (85% yield) as a white solid: ¹H NMR (400 MHz, CD₃OD) δ 7.72 (d, J=8.5 Hz, 2H), 7.61-7.55 (m, 2H), 7.41 (d, J=8.5 Hz, 2H), 7.30-7.14 (m, 5), 7.04-6.96 (m, 2H), 4.91 (q, J=7.1 Hz, 1H), 4.03 (dd, J=15.1, 7.5 Hz, 1H), 3.84-3.75 (m, 4H), 3.42 (p, J=6.2 Hz, 1), 3.26 (s, 3H), 3.19-3.10 (m, 1H), 1.33 (d, J=7.1 Hz, 3H), 1.08 (d, J=6.2 Hz, 3H); ¹³C NMR (100 MHz, CD₃OD) δ 159.9, 158.3, 144.6, 140.8, 139.8, 132.0, 128.2, 128.1, 128.1, 127.8, 126.6, 125.6, 114.1, 73.6, 56.4, 55.4, 54.4, 50.6, 50.0, 21.3, 14.2; MS (ESI) m/z 448.40 [M+H]⁺; HPLC, >99%, t_{R} 14.5 min.

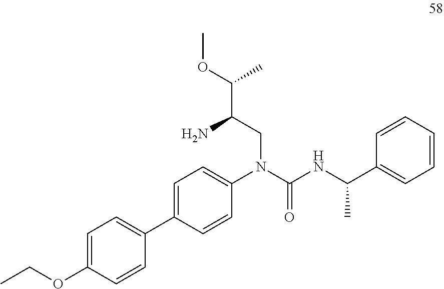 [SMILES: CCOc1ccc(-c2ccc(N(C[C@@H](N)[C@@H](C)OC)C(=O)N[C@@H](C)c3ccccc3)cc2)cc1]

3-[(2R,3R)-2-Amino-3-methoxybutyl]-1-[(1 S)-1-phenylethyl]-3-{4′-ethoxy-[1,1′-biphenyl]-4-yl}urea (58). The procedure for the synthesis of 55 was followed starting with E3r and 4-ethoxyphenylboronic acid to afford the Boc-protected biphenyl urea E4d (69% yield), which was then deprotected with HCl to give 58 hydrochloride salt (80% yield) as a white solid: ¹H NMR (300 MHz, CD₃OD) δ 7.81-7.69 (m, 2H), 7.60 (d, J=7.8 Hz, 2H), 7.51-7.39 (m, 3H), 7.32-7.11 (m, 6H), 7.01 (d, J=7.3 Hz, 2H), 4.97-4.90 (m, 1H), 4.08 (q, J=6.7 Hz, 3H), 3.82 (d, J=14.0 Hz, 1H), 3.45 (d, J=7.0 Hz, 1H), 3.21 (s, 1H), 1.55-1.20 (m, 7H), 1.18-1.03 (m, 3H), 0.93 (dd, J=26.8, 7.0 Hz, 1H); ¹³C NMR (75 MHz, CD₃OD) δ 160.5, 159.7, 145.9, 142.3, 141.2, 133.3, 129.6, 129.5, 129.2, 128.0, 127.0, 116.1, 75.0, 64.7, 57.9, 56.9, 52.0, 51.5, 22.8, 15.8, 15.2; MS (ESI) m/z 462.20 [M+H]⁺; HPLC, >98%, t_{R} 15.2 min.

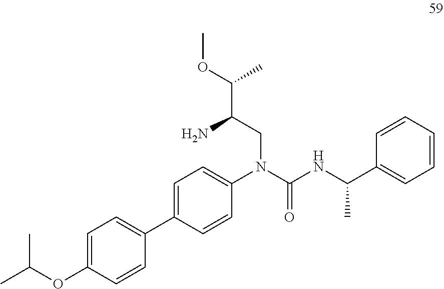 [SMILES: CO[C@H](C)[C@H](N)CN(C(=O)N[C@@H](C)c1ccccc1)c1ccc(-c2ccc(OC(C)C)cc2)cc1]

3-[(2R,3R)-2-Amino-3-methoxybutyl]-1-[(1 S)-1-phenylethyl]-3-[4′-(propan-2-yloxy)-[1,1′-biphenyl]-4-yl]urea (59). The procedure for the synthesis of 55 was followed starting with E3r and 4-isoproxyphenylboronic acid to afford the Boc-protected biphenyl urea E4e (69% yield), which was then deprotected with HCl to give 59 hydrochloride salt (80% yield) as a white solid: ¹H NMR (400 MHz, CD₃OD) δ 7.72 (d, J=8.2 Hz, 2H), 7.56 (d, J=8.5 Hz, 2H), 7.40 (d, J=8.3 Hz, 2H), 7.32-7.10 (m, 5H), 6.97 (d, J=8.6 Hz, 2H), 4.91 (q, J=7.0 Hz, 1H), 4.70-4.55 (m, 1H), 4.03 (dd, J=15.2, 7.5 Hz, 1H), 3.80 (dd, J=15.1, 3.9 Hz, 1H), 3.48-3.35 (m, 1H), 3.26 (s, 3H), 3.21-3.10 (m, 1H), 1.39-1.24 (m, 9H), 1.08 (d, J=6.2 Hz, 3H); ¹³C NMR (100 MHz, CD₃OD) δ 158.3, 158.0, 144.6, 140.9, 139.8, 131.9, 128.1, 128.1, 127.8, 126.6, 125.6, 116.0, 73.6, 69.7, 56.4, 55.4, 50.6, 50.0, 21.3, 21.0, 14.2; MS (ESI) m/z 476.40 [M+H]⁺: HPLC, >99%, t_{R} 15.7 min.

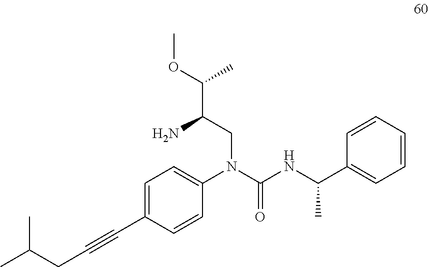 [SMILES: CO[C@H](C)[C@H](N)CN(C(=O)N[C@@H](C)c1ccccc1)c1ccc(C#CCC(C)C)cc1]

3-[(2R,3R)-2-amino-3-methoxybutyl]-3-[4-(4-methylpent-1-yn-1-yl)phenyl]-1-[(1 S)-1-phenylethyl]urea (60). Sonogashira coupling of E3r and 4-methyl-1-pent-1-yne was carried out following the procedure used for the synthesis of 36 to afford the Boc-protected (4-methylpent-1-yn-1-yl)phenyl urea E4f (75% yield), which was deprotected with HCl to give 60 hydrochloride salt (75% yield) as a white solid: ¹H NMR (400 MHz, CD₃OD) δ 7.48 (d, J=8.5 Hz, 2H), 7.33-7.11 (m, 7H), 4.92-4.87 (m, 1H), 3.98 (dd, J=15.2, 7.4 Hz, 1H), 3.76 (dd, J=15.2, 4.0 Hz, 1H), 3.38 (p, J=6.2 Hz, 1H), 3.25 (s, 3H), 3.18-3.05 (m, 1H), 2.30 (d, J=6.5 Hz, 2H), 1.95-1.80 (m, 1H), 1.32 (d, J=7.1 Hz, 3H), 1.11-0.98 (m, 9H); ¹³C NMR (100 MHz, CD₃OD) δ 158.0, 144.6, 140.5, 133.1, 128.1, 127.7, 126.6, 125.6, 124.2, 90.1, 73.5, 56.4, 55.4, 50.6, 49.8, 28.0, 27.8, 21.2, 21.0, 14.1; MS (ESI) m/z 422.40 [M+H]⁺; HPLC, >99%, t_{R} 15.9 min.

### Pharmacology

Materials. Cell culture materials were purchased from Fisher SSI. Forskolin was purchased from Sigma-Aldrich. The Lance Ultra kit (TRF0262) was purchased from PerkinElmer.

Lance™ Ultra cAMP assay using stable PPLS-HA-GPR88 CHO cells. All cAMP assays were performed using the inventors' previously published methods. See, e.g., Jin et al., Effect of substitution on the aniline moiety of the GPR88 agonist 2-PCCA: synthesis, structure-activity relationships, and molecular modeling studies. ACS Chemical Neuroscience 2016, 7, 1418-1432, which is incorporated by reference herein with regard to such teaching. Stimulation buffer containing 1× Hank's Balanced Salt Solution (HBSS), 5 mM HEPES, 0.1% BSA stabilizer, and 0.5 mM final IBMX was prepared and titrated to pH 7.4 at room temperature. Serial dilutions of the test compounds (5 μL) and 300 nM forskolin (5 μL), both prepared at 4× the desired final concentration in 2% DMSO/stimulation buffer, were added to a 96-well white ½ area microplate (PerkinElmer). A cAMP standard curve was prepared at 4× the desired final concentration in stimulation buffer and 5 μL was added to the assay plate. Stable PPLS-HA-GPR88 CHO cells were lifted with versene and spun at 270 g for 10 minutes. The cell pellet was resuspended in stimulation buffer and 4,000 cells (10 μL) were added to each well except wells containing the cAMP standard curve. After incubating for 30 min at room temperature, Eu-cAMP tracer and uLIGHT-anti-cAMPworking solutions were added per the manufacturer's instructions. After incubation at room temperature for 1 hour, the TR-FRET signal (ex 337 nm) was read on a CLARIOstar multimode plate reader (BMG Biotech, Cary, NC).

Data Analysis. The TR-FRET signal (665 nm) was converted to fmol cAMP by interpolating from the standard cAMP curve. Fmol cAMP was plotted against the log of compound concentration and data were fit to a three-parameter logistic curve to generate EC₅₀ values (Prism, version 6.0, GraphPad Software, Inc., San Diego, CA). The Eₘₐₓ value for each test compound relative to the control compound RTI-13951-33 was calculated with the equation % control Eₘₐₓ=(maximal test compound signal/maximal control signal)×100.

TABLE 1
Biological data for select GPR88 agonists of the present disclosure

| | | cAMP pEC₅₀ (EC₅₀, | |
|---|---|---|---|
| Compound | Structure | nM)^{a} | Eₘₐₓ (%)^{b} |
| 3 | 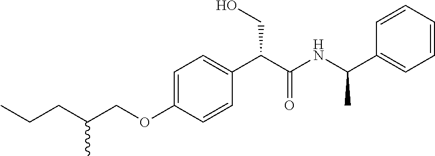 [SMILES: CCCC(C)COc1ccc([C@H](CO)C(=O)N[C@H](C)c2ccccc2)cc1] | 5.46 ± 0.05 (3467) | 77 ± 4 |
| 4 | 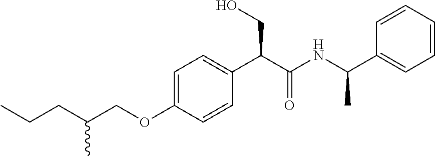 [SMILES: CCCC(C)COc1ccc([C@@H](CO)C(=O)N[C@H](C)c2ccccc2)cc1] | 6.70 ± 0.05 (200) | 94 ± 13 |
| 5 | 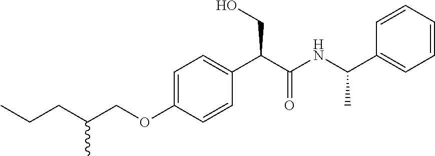 [SMILES: CCCC(C)COc1ccc([C@@H](CO)C(=O)N[C@@H](C)c2ccccc2)cc1] | Inactive | Not determined |
| 6 | 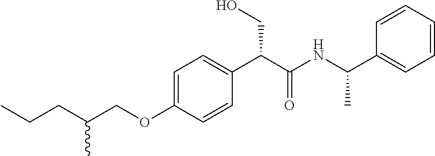 [SMILES: CCCC(C)COc1ccc([C@H](CO)C(=O)N[C@@H](C)c2ccccc2)cc1] | Inactive | Not determined |
| 7 | 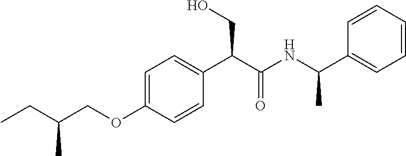 [SMILES: CC[C@H](C)COc1ccc([C@@H](CO)C(=O)N[C@H](C)c2ccccc2)cc1] | 5.70 ± 0.12 (1995) | 88 ± 7 |
| 8 | 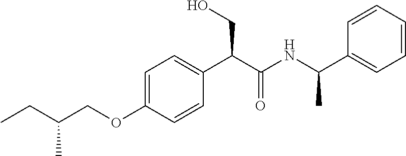 [SMILES: CC[C@@H](C)COc1ccc([C@@H](CO)C(=O)N[C@H](C)c2ccccc2)cc1] | 5.51 ± 0.15 (3090) | 96 ± 13 |
| 9 | 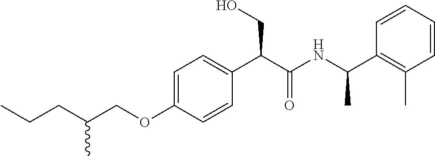 [SMILES: CCCC(C)COc1ccc([C@@H](CO)C(=O)N[C@H](C)c2ccccc2C)cc1] | 5.28 ± 0.13 (5248) | 48 ± 8 |
| 10 | 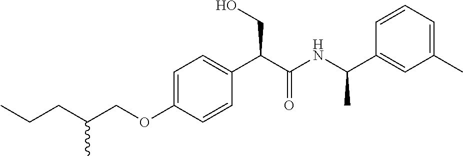 [SMILES: CCCC(C)COc1ccc([C@@H](CO)C(=O)N[C@H](C)c2cccc(C)c2)cc1] | 6.45 ± 0.04 (355) | 100 ± 3 |
| 11 | 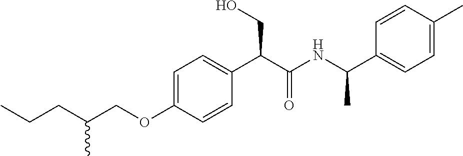 [SMILES: CCCC(C)COc1ccc([C@@H](CO)C(=O)N[C@H](C)c2ccc(C)cc2)cc1] | 6.83 ± 0.07 (148) | 102 ± 7 |
| 12 | 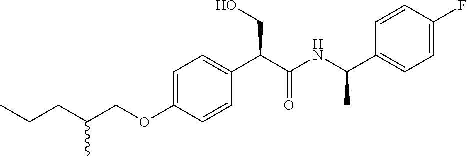 [SMILES: CCCC(C)COc1ccc([C@@H](CO)C(=O)N[C@H](C)c2ccc(F)cc2)cc1] | 6.05 ± 0.05 (891) | 92 ± 3 |
| 13 | 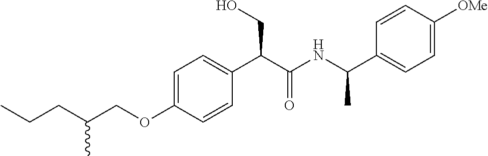 [SMILES: CCCC(C)COc1ccc([C@@H](CO)C(=O)N[C@H](C)c2ccc(OC)cc2)cc1] | 6.13 ± 0.11 (741) | 94 ± 3 |
| 14 | 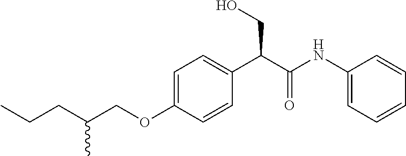 [SMILES: CCCC(C)COc1ccc([C@@H](CO)C(=O)Nc2ccccc2)cc1] | Inactive | Not determined |
| 15 | 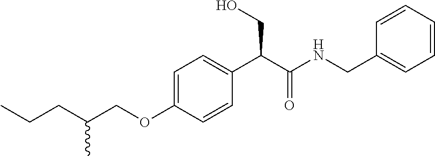 [SMILES: CCCC(C)COc1ccc([C@@H](CO)C(=O)NCc2ccccc2)cc1] | 6.23 ± 0.09 (589) | 99 ± 9 |
| 16 | 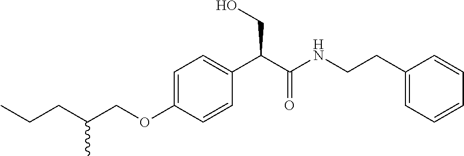 [SMILES: CCCC(C)COc1ccc([C@@H](CO)C(=O)NCCc2ccccc2)cc1] | 6.15 ± 0.06 (708) | 97 ± 5 |
| 17 | 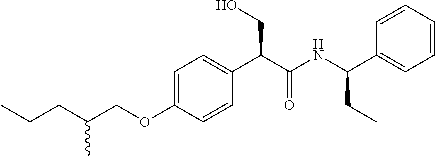 [SMILES: CCCC(C)COc1ccc([C@@H](CO)C(=O)N[C@H](CC)c2ccccc2)cc1] | 6.44 ± 0.04 (363) | 102 ± 8 |
| 18 | 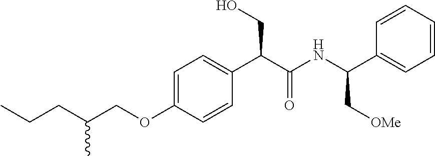 [SMILES: CCCC(C)COc1ccc([C@@H](CO)C(=O)N[C@H](COC)c2ccccc2)cc1] | 6.29 ± 0.05 (513) | 91 ± 4 |
| 19 | 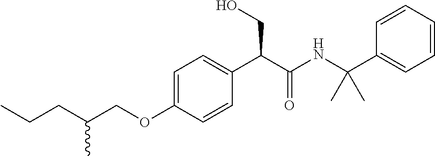 [SMILES: CCCC(C)COc1ccc([C@@H](CO)C(=O)NC(C)(C)c2ccccc2)cc1] | 5.90 ± 0.05 (1259) | 83 ± 2 |
| 20 | 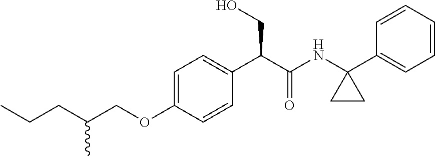 [SMILES: CCCC(C)COc1ccc([C@@H](CO)C(=O)NC2(c3ccccc3)CC2)cc1] | 5.72 ± 0.08 (1905) | 81 ± 8 |
| 21 | 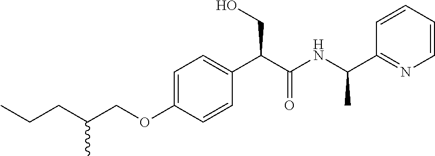 [SMILES: CCCC(C)COc1ccc([C@@H](CO)C(=O)N[C@H](C)c2ccccn2)cc1] | 5.41 ± 0.10 (3890) | 93 ± 4 |
| 22 | 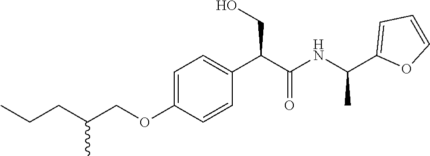 [SMILES: CCCC(C)COc1ccc([C@@H](CO)C(=O)N[C@H](C)c2ccco2)cc1] | 5.99 ± 0.09 (1023) | 108 ± 6 |
| 23 | 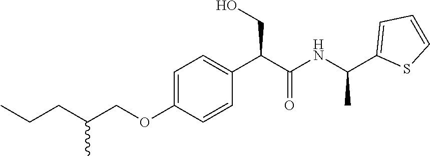 [SMILES: CCCC(C)COc1ccc([C@@H](CO)C(=O)N[C@H](C)c2cccs2)cc1] | 6.29 ± 0.08 (513) | 93 ± 5 |
| 24 | 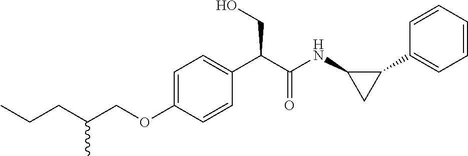 [SMILES: CCCC(C)COc1ccc([C@@H](CO)C(=O)N[C@@H]2C[C@H]2c2ccccc2)cc1] | 5.39 ± 0.09 (4074) | 83 ± 5 |
| 25 | 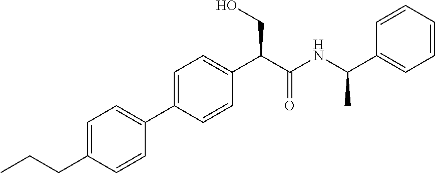 [SMILES: CCCc1ccc(-c2ccc([C@@H](CO)C(=O)N[C@H](C)c3ccccc3)cc2)cc1] | 6.86 ± 0.09 (138) | 96 ± 3 |
| 26 | 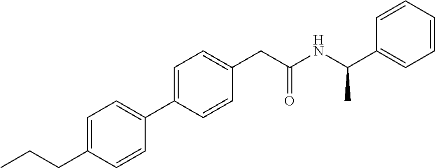 [SMILES: CCCc1ccc(-c2ccc(CC(=O)N[C@H](C)c3ccccc3)cc2)cc1] | 6.59 ± 0.10 (257) | 95 ± 5 |
| 27 | 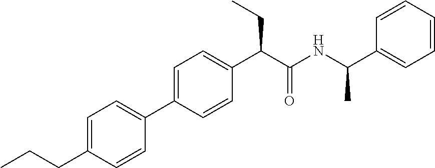 [SMILES: CCCc1ccc(-c2ccc([C@@H](CC)C(=O)N[C@H](C)c3ccccc3)cc2)cc1] | 6.30 ± 0.03 (501) | 93 ± 9 |
| 28 | 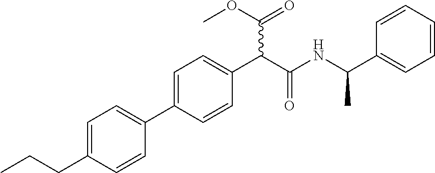 [SMILES: CCCc1ccc(-c2ccc(C(C(=O)N[C@H](C)c3ccccc3)C(=O)OC)cc2)cc1] | 5.89 ± 0.03 (1288) | 99 ± 6 |
| 29 | 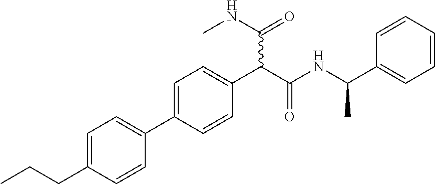 [SMILES: CCCc1ccc(-c2ccc(C(C(=O)NC)C(=O)N[C@H](C)c3ccccc3)cc2)cc1] | 5.86 ± 0.06 (1380) | 105 ± 2 |
| 30 | 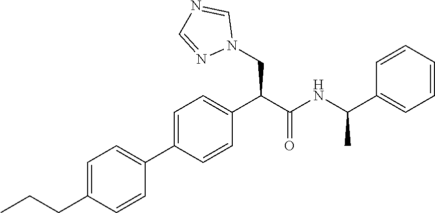 [SMILES: CCCc1ccc(-c2ccc([C@@H](Cn3cncn3)C(=O)N[C@H](C)c3ccccc3)cc2)cc1] | 5.96 ± 0.03 (1096) | 96 ± 3 |
| 31 | 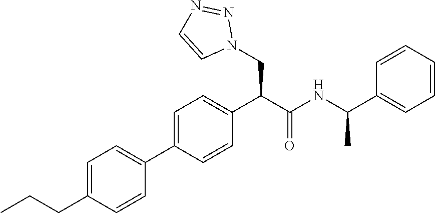 [SMILES: CCCc1ccc(-c2ccc([C@@H](Cn3ccnn3)C(=O)N[C@H](C)c3ccccc3)cc2)cc1] | 6.20 ± 0.06 (631) | 96 ± 0 |
| 32 | 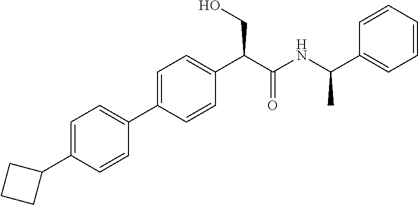 [SMILES: C[C@@H](NC(=O)[C@H](CO)c1ccc(-c2ccc(C3CCC3)cc2)cc1)c1ccccc1] | 6.45 ± 0.04 (355) | 74 ± 7 |
| 33 | 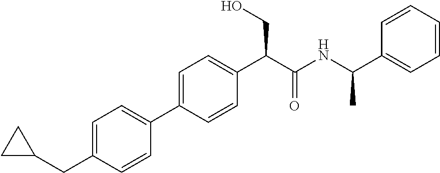 [SMILES: C[C@@H](NC(=O)[C@H](CO)c1ccc(-c2ccc(CC3CC3)cc2)cc1)c1ccccc1] | 6.58 ± 0.06 (263) | 87 ± 5 |
| 34 | 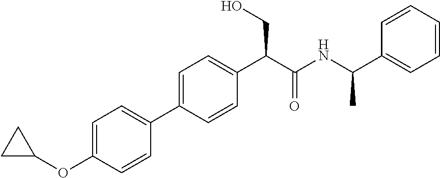 [SMILES: C[C@@H](NC(=O)[C@H](CO)c1ccc(-c2ccc(OC3CC3)cc2)cc1)c1ccccc1] | 6.61 ± 0.10 (245) | 86 +4 |
| 35 | 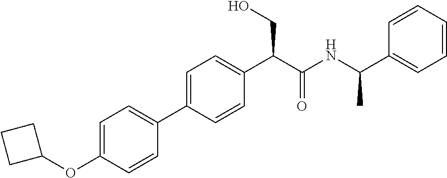 [SMILES: C[C@@H](NC(=O)[C@H](CO)c1ccc(-c2ccc(OC3CCC3)cc2)cc1)c1ccccc1] | 6.65 ± 0.08 (224) | 90 ± 10 |
| 36 | 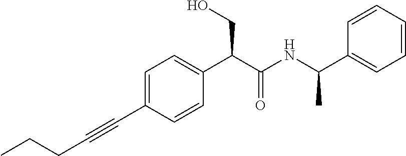 [SMILES: CCCC#Cc1ccc([C@@H](CO)C(=O)N[C@H](C)c2ccccc2)cc1] | 6.69 ± 0.02 (204) | 92 ± 7 |
| 37 | 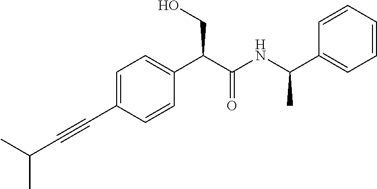 [SMILES: CC(C)C#Cc1ccc([C@@H](CO)C(=O)N[C@H](C)c2ccccc2)cc1] | 6.32 ± 0.03 (479) | 102 ± 2 |
| 38 | 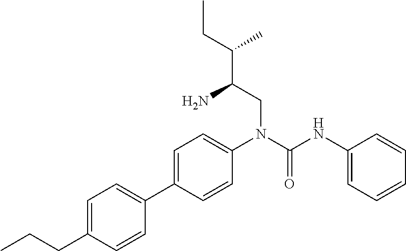 [SMILES: CCCc1ccc(-c2ccc(N(C[C@@H](N)[C@@H](C)CC)C(=O)Nc3ccccc3)cc2)cc1] | Inactive | Not determined |
| 39 | 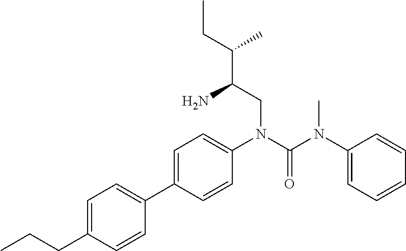 [SMILES: CCCc1ccc(-c2ccc(N(C[C@@H](N)[C@@H](C)CC)C(=O)N(C)c3ccccc3)cc2)cc1] | 5.55 ± 0.02 (2818) | 97 ± 12 |
| 40 | 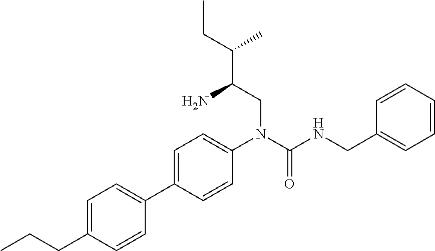 [SMILES: CCCc1ccc(-c2ccc(N(C[C@@H](N)[C@@H](C)CC)C(=O)NCc3ccccc3)cc2)cc1] | 5.70 ± 0.02 (1995) | 97 ± 4 |
| 41 | 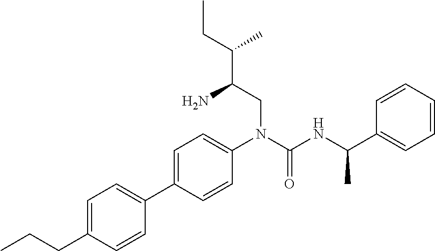 [SMILES: CCCc1ccc(-c2ccc(N(C[C@@H](N)[C@@H](C)CC)C(=O)N[C@H](C)c3ccccc3)cc2)cc1] | Inactive | Not determined |
| 42 | 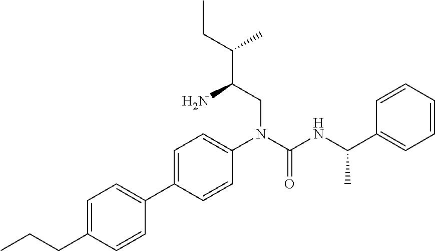 [SMILES: CCCc1ccc(-c2ccc(N(C[C@@H](N)[C@@H](C)CC)C(=O)N[C@@H](C)c3ccccc3)cc2)cc1] | 5.99 ± 0.03 (1023) | 106 ± 10 |
| 43 | 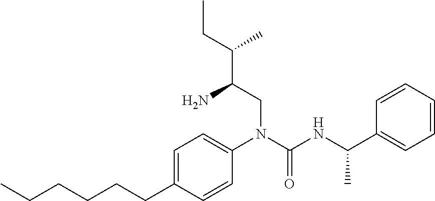 [SMILES: CCCCCCc1ccc(N(C[C@@H](N)[C@@H](C)CC)C(=O)N[C@@H](C)c2ccccc2)cc1] | 5.86 ± 0.07 (1380) | 108 ± 4 |
| 44 | 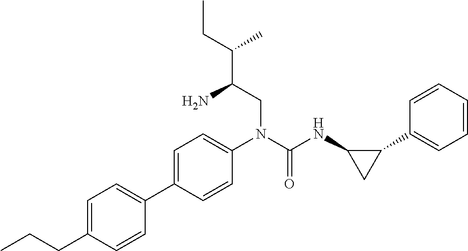 [SMILES: CCCc1ccc(-c2ccc(N(C[C@@H](N)[C@@H](C)CC)C(=O)N[C@@H]3C[C@H]3c3ccccc3)cc2)cc1] | Inactive | Not determined |
| 45 | 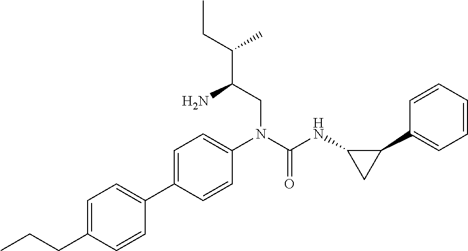 [SMILES: CCCc1ccc(-c2ccc(N(C[C@@H](N)[C@@H](C)CC)C(=O)N[C@H]3C[C@@H]3c3ccccc3)cc2)cc1] | 5.64 ± 0.05 (2291) | 110 ± 5 |
| 46 | 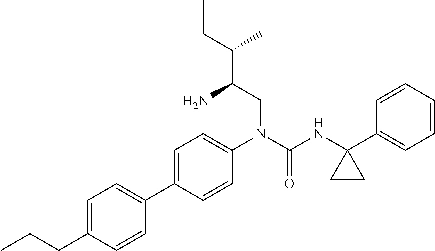 [SMILES: CCCc1ccc(-c2ccc(N(C[C@@H](N)[C@@H](C)CC)C(=O)NC3(c4ccccc4)CC3)cc2)cc1] | 5.73 ± 0.07 (1862) | 91 ± 4 |
| 47 | 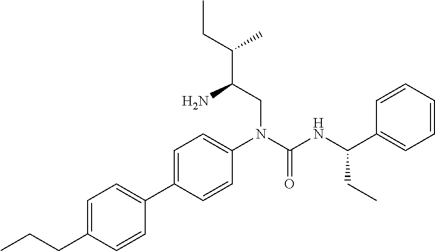 [SMILES: CCCc1ccc(-c2ccc(N(C[C@@H](N)[C@@H](C)CC)C(=O)N[C@@H](CC)c3ccccc3)cc2)cc1] | 5.70 ± 0.06 (1995) | 103 ± 4 |
| 48 | 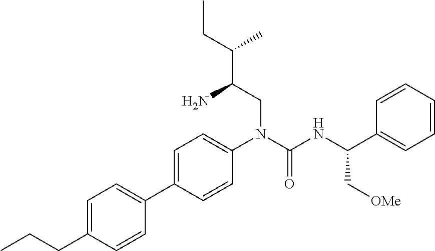 [SMILES: CCCc1ccc(-c2ccc(N(C[C@@H](N)[C@@H](C)CC)C(=O)N[C@@H](COC)c3ccccc3)cc2)cc1] | 5.81 ± 0.05 (1549) | 110 ± 5 |
| 49 | 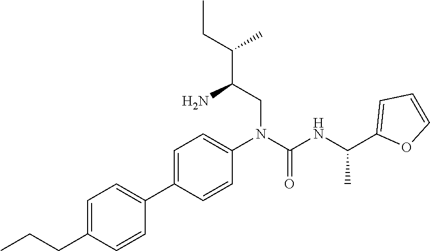 [SMILES: CCCc1ccc(-c2ccc(N(C[C@@H](N)[C@@H](C)CC)C(=O)N[C@@H](C)c3ccco3)cc2)cc1] | 5.59 ± 0.06 (2570) | 115 ± 5 |
| 50 | 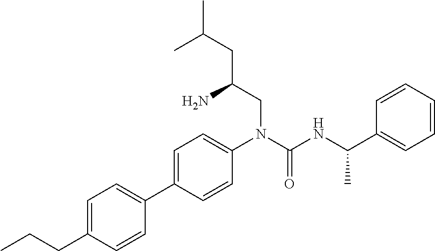 [SMILES: CCCc1ccc(-c2ccc(N(C[C@@H](N)CC(C)C)C(=O)N[C@@H](C)c3ccccc3)cc2)cc1] | 5.92 ± 0.05 (1202) | 112 ± 8 |
| 51 | 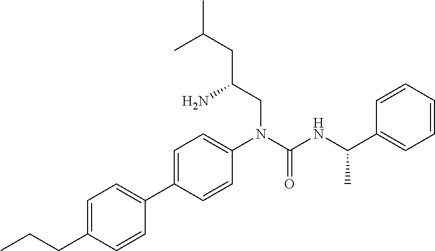 [SMILES: CCCc1ccc(-c2ccc(N(C[C@H](N)CC(C)C)C(=O)N[C@@H](C)c3ccccc3)cc2)cc1] | 5.85 ± 0.04 (1413) | 129 ± 4 |
| 52 | 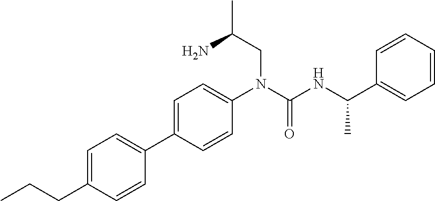 [SMILES: CCCc1ccc(-c2ccc(N(C[C@H](C)N)C(=O)N[C@@H](C)c3ccccc3)cc2)cc1] | 5.89 ± 0.02 (1288) | 107 ± 3 |
| 53 | 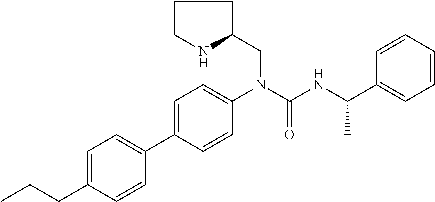 [SMILES: CCCc1ccc(-c2ccc(N(C[C@@H]3CCCN3)C(=O)N[C@@H](C)c3ccccc3)cc2)cc1] | 5.76 ± 0.05 (1738) | 106 ± 3 |
| 54 | 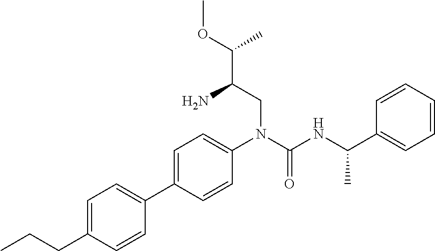 [SMILES: CCCc1ccc(-c2ccc(N(C[C@@H](N)[C@@H](C)OC)C(=O)N[C@@H](C)c3ccccc3)cc2)cc1] | 6.49 ± 0.03 (324) | 106 ± 3 |
| 55 | 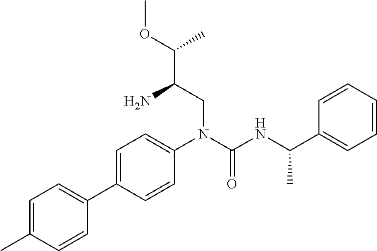 [SMILES: CO[C@H](C)[C@H](N)CN(C(=O)N[C@@H](C)c1ccccc1)c1ccc(-c2ccc(C)cc2)cc1] | 6.34 ± 0.06 (457) | 114 ± 6 |
| 56 | 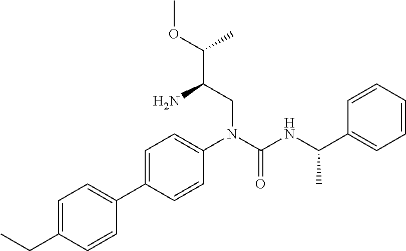 [SMILES: CCc1ccc(-c2ccc(N(C[C@@H](N)[C@@H](C)OC)C(=O)N[C@@H](C)c3ccccc3)cc2)cc1] | 6.48 ± 0.06 (331) | 116 ± 9 |
| 57 | 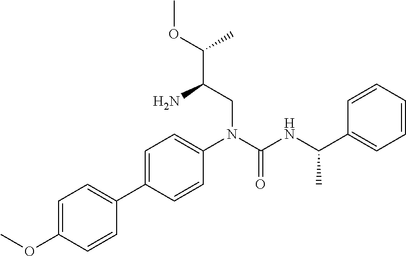 [SMILES: COc1ccc(-c2ccc(N(C[C@@H](N)[C@@H](C)OC)C(=O)N[C@@H](C)c3ccccc3)cc2)cc1] | 6.05 ± 0.06 (891) | 101 ± 3 |
| 58 | 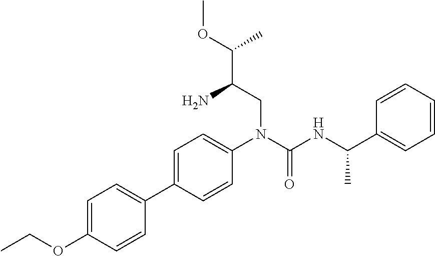 [SMILES: CCOc1ccc(-c2ccc(N(C[C@@H](N)[C@@H](C)OC)C(=O)N[C@@H](C)c3ccccc3)cc2)cc1] | 6.17 ± 0.03 (676) | 102 ± 2 |
| 59 | 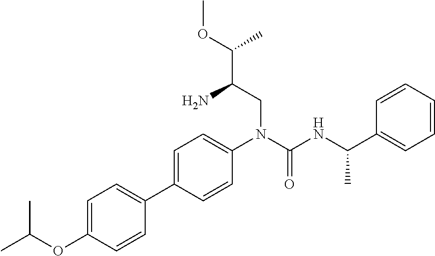 [SMILES: CO[C@H](C)[C@H](N)CN(C(=O)N[C@@H](C)c1ccccc1)c1ccc(-c2ccc(OC(C)C)cc2)cc1] | 6.39 ± 0.04 (407) | 100 ± 4 |
| 60 | 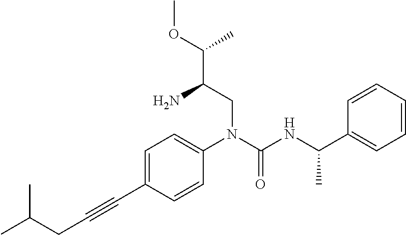 [SMILES: CO[C@H](C)[C@H](N)CN(C(=O)N[C@@H](C)c1ccccc1)c1ccc(C#CCC(C)C)cc1] | 6.44 ± 0.06 (363) | 103 ± 4 |

^{a}pEC₅₀ values are means ± standard error of at least three independent experiments performed in duplicate.
^{b}Eₘₐₓ value is % of RTI-13951-33 maximal signal (mean ± standard error).

References, wherein each is incorporated with regard to such respective background teaching:
1. Rehm, J.; Mathers, C.; Popova, S.; Thavorncharoensap, M.; Teerawattananon, Y.; Patra, J., Global burden of disease and injury and economic cost attributable to alcohol use and alcohol-use disorders. Lancet 2009, 373, 2223-2233.
2. Thavorncharoensap, M.; Teerawattananon, Y.; Yothasamut, J.; Lertpitakpong, C.; Chaikledkaew, U., The economic impact of alcohol consumption: a systematic review. Substance abuse treatment, prevention, and policy 2009, 4, 20.
3. Costardi, J. V.; Nampo, R. A.; Silva, G. L.; Ribeiro, M. A.; Stella, H. J.; Stella, M. B.; Malheiros, S. V., A review on alcohol: from the central action mechanism to chemical dependency. Revista da Associacao Medica Brasileira 2015, 61, 381-387.
4. Bouza, C.; Angeles, M.; Munoz, A.; Amate, J. M., Efficacy and safety of naltrexone and acamprosate in the treatment of alcohol dependence: a systematic review. Addiction 2004, 99, 811-828.
5. Mizushima, K.; Miyamoto, Y.; Tsukahara, F.; Hirai, M.; Sakaki, Y.; Ito, T., A novel G-protein-coupled receptor gene expressed in striatum. Genomics 2000, 69, 314-321.
6. Van Waes, V.; Tseng, K. Y.; Steiner, H., GPR88—a putative signaling molecule predominantly expressed in the striatum: Cellular localization and developmental regulation. Basal Ganglia 2011, 1, 83-89.
7. Massart, R.; Guilloux, J. P.; Mignon, V.; Sokoloff, P.; Diaz, J., Striatal GPR88 expression is confined to the whole projection neuron population and is regulated by dopaminergic and glutamatergic afferents. European Journal of Neuroscience 2009, 30, 397-414.
8. Ye, N.; Li, B.; Mao, Q.; Wold, E. A.; Tian, S.; Allen, J. A.; Zhou, J., Orphan receptor GPR88 as an emerging neurotherapeutic target. ACS Chemical Neuroscience 2019, 10, 190-200.
9. Logue, S. F.; Grauer, S. M.; Paulsen, J.; Graf, R.; Taylor, N.; Sung, M. A.; Zhang, L.; Hughes, Z.; Pulito, V. L.; Liu, F.; Rosenzweig-Lipson, S.; Brandon, N. J.; Marquis, K. L.; Bates, B.; Pausch, M., The orphan GPCR, GPR88, modulates function of the striatal dopamine system: a possible therapeutic target for psychiatric disorders?Molecular and Cellular Neuroscience 2009, 42, 438-447.
10. Quintana, A.; Sanz, E.; Wang, W.; Storey, G. P.; Guler, A. D.; Wanat, M. J.; Roller, B. A.; La Torre, A.; Amieux, P. S.; McKnight, G. S.; Bamford, N. S.; Palmiter, R. D., Lack of GPR88 enhances medium spiny neuron activity and alters motor- and cue-dependent behaviors. Nature Neuroscience 2012, 15, 1547-1555.
11. Ben Hamida, S.; Mendonca-Netto, S.; Arefin, T. M.; Nasseef, M. T.; Boulos, L. J.; McNicholas, M.; Ehrlich, A. T.; Clarke, E.; Moquin, L.; Gratton, A.; Darcq, E.; Harsan, L. A.; Maldonado, R.; Kieffer, B. L., Increased alcohol seeking in mice lacking Gpr88 involves dysfunctional mesocorticolimbic networks. Biological Psychiatry 2018, 84, 202-212.
12. Jin, C.; Decker, A. M.; Makhijani, V. H.; Besheer, J.; Darcq, E.; Kieffer, B. L.; Maitra, R., Discovery of a potent, selective, and brain-penetrant small molecule that activates the orphan receptor GPR88 and reduces alcohol intake. Journal of Medicinal Chemistry 2018, 61, 6748-6758.
13. Dzierba, C. D.; Bi, Y.; Dasgupta, B.; Hartz, R. A.; Ahuja, V.; Cianchetta, G.; Kumi, G.; Dong, L.; Aleem, S.; Fink, C.; Garcia, Y.; Green, M.; Han, J.; Kwon, S.; Qiao, Y.; Wang, J.; Zhang, Y.; Liu, Y.; Zipp, G.; Liang, Z.; Burford, N.; Ferrante, M.; Bertekap, R.; Lewis, M.; Cacace, A.; Grace, J.; Wilson, A.; Nouraldeen, A.; Westphal, R.; Kimball, D.; Carson, K.; Bronson, J. J.; Macor, J. E., Design, synthesis, and evaluation of phenylglycinols and phenyl amines as agonists of GPR88. Bioorgic & Medicinal Chemistry Letters 2015, 25, 1448-1452.
14. Jin, C.; Decker, A. M.; Langston, T. L., Design, synthesis and pharmacological evaluation of 4-hydroxyphenylglycine and 4-hydroxyphenylglycinol derivatives as GPR88 agonists. Bioorganic & Medicinal Chemistry 2017, 25, 805-812.
15. Rahman, M. T.; Decker, A. M.; Langston, T. L.; Mathews, K. M.; Laudermilk, L.; Maitra, R.; Ma, W.; Darcq, E.; Kieffer, B. L.; Jin, C., Design, synthesis, and structure-activity relationship studies of (4-alkoxyphenyl)glycinamides and bioisosteric 1,3,4-oxadiazoles as GPR88 agonists. Journal of Medicinal Chemistry 2020, 63, 14989-15012.
16. Rahman, M. T.; Decker, A. M.; Laudermilk, L.; Maitra, R.; Ma, W.; Ben Hamida, S.; Darcq, E.; Kieffer, B. L.; Jin, C., Evaluation of amide bioisosteres leading to 1,2,3-triazole containing compounds as GPR88 agonists: Design, synthesis, and structure-activity relationship studies. Journal of Medicinal Chemistry 2021, 64, 12397-12413.
17. Jin, C.; Decker, A. M.; Harris, D. L.; Blough, B. E., Effect of substitution on the aniline moiety of the GPR88 agonist 2-PCCA: Synthesis, structure-activity relationships, and molecular modeling studies. ACS Chemical Neuroscience 2016, 7, 1418-1432.

The specific pharmacological responses observed may vary according to and depending on the particular active compound selected or whether there are present pharmaceutical carriers, as well as the type of formulation and mode of administration employed, and such expected variations or differences in the results are contemplated in accordance with practice of the present disclosure.

Although specific embodiments of the present disclosure are herein illustrated and described in detail, the disclosure is not limited thereto. The above detailed descriptions are provided as exemplary of the present disclosure and should not be construed as constituting any limitation of the disclosure. Modifications will be obvious to those skilled in the art, and all modifications that do not depart from the spirit of the disclosure are intended to be included with the scope of the appended claims.

1. A compound of Formula (I) or a pharmaceutically acceptable salt thereof: 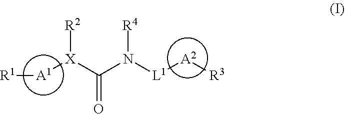
wherein
R¹ is selected from the group consisting of hydrogen, C₁₋₁₀ alkyl, C₂₋₁₀ alkenyl, C₂₋₁₀ alkynyl, C₁₋₁₀ alkoxy, C₁₋₁₀ haloalkyl, NH₂, NHC₁₋₁₀ alkyl, N(C₁₋₁₀ alkyl)₂, S(O)C₁₋₁₀ alkyl, S(O)₂C₁₋₁₀ alkyl, NHSO₂C₁₋₁₀ alkyl, C₃₋₆ cycloalkyl, 5-7 membered heterocycle, C₆₋₁₀ aryl, and C₅₋₁₀ heteroaryl;
A¹ is
a) a 6-membered aromatic ring which may contain one N heteroatom; or
b) a 10-membered fused aromatic ring system which may contain one or two N heteroatoms;
X is CH or N;
R² is (CH₂)ₙ₂—R²⁰;
n2 is 0, 1, 2, or 3;
R²⁰ is selected from the group consisting of hydrogen, C₁₋₆ alkyl, C₂₋₆ alkenyl, C₂₋₆ alkynyl, OH, C₁₋₆ alkoxy, halogen, C₁₋₆ haloalkyl, NO₂, CN, NH₂, NHC₁₋₆ alkyl, N(C₁₋₆ alkyl)₂, N₃, S(O)C₁₋₆ alkyl, S(O)₂C₁₋₆ alkyl, NHSO₂C₁₋₆ alkyl, C(O)C₁₋₆ alkyl, C(O)OC₁₋₆ alkyl, NHC(O)C₁₋₆ alkyl, N(C₁₋₆ alkyl)C(O)C₁₋₆ alkyl, C(O)NHC₁₋₆ alkyl, C(O)N(C₁₋₆ alkyl)₂, CH(NH₂)C₁₋₆alkyl, CH(N[C₁₋₆ alkyl]₂)C₁₋₆alkyl, CH(NH₂)C₁₋₆alkylene-O—C₁₋₆alkyl, CH(N[C₁₋₆ alkyl]₂)C₁₋₆alkylene-O—C₁₋₆alkyl, C₃₋₆ cycloalkyl, 5-7 membered heterocycle, C₆₋₁₀ aryl, and C₅₋₁₀ heteroaryl;
R⁴ is (CH₂)ₙ₄—R⁴⁰;
n4 is 0, 1, 2, or 3;
R⁴⁰ is selected from the group consisting of hydrogen, C₁₋₆ alkyl, C₂₋₆ alkenyl, C₂₋₆ alkynyl, OH, C₁₋₆ alkoxy, halogen, C₁₋₆ haloalkyl, NO₂, CN, NH₂, NHC₁₋₆ alkyl, N(C₁₋₆ alkyl)₂, N₃, S(O)C₁₋₆ alkyl, S(O)₂C₁₋₆ alkyl, NHSO₂C₁₋₆ alkyl, C(O)C₁₋₆ alkyl, C(O)OC₁₋₆ alkyl, NHC(O)C₁₋₆ alkyl, N(C₁₋₆ alkyl)C(O)C₁₋₆ alkyl, C(O)NHC₁₋₆ alkyl, C(O)N(C₁₋₆ alkyl)₂, CH(NH₂)C₁₋₆alkyl, CH(N[C₁₋₆ alkyl]₂)C₁₋₆alkyl, CH(NH₂)C₁₋₆alkylene-O—C₁₋₆alkyl, CH(N[C₁₋₆ alkyl]₂)C₁₋₆alkylene-O—C₁₋₆alkyl, C₃₋₆ cycloalkyl, 5-7 membered heterocycle, C₆₋₁₀ aryl, and C₅₋₁₀ heteroaryl;
L¹ is
a) divalent C₃ cyclolakyl;
b) [(CR^{L})₂]ₘ; or
c) a bond
m is 0, 1, 2, or 3
each R^{L} independently is selected from the group consisting of hydrogen, C₁₋₆ alkyl, C₂₋₆ alkenyl, C₂₋₆ alkynyl, OH, C₁₋₆ alkoxy, halogen, C₁₋₆ haloalkyl, NO₂, CN, NH₂, NHC₁₋₆ alkyl, N(C₁₋₆ alkyl)₂, N₃, S(O)C₁₋₆ alkyl, S(O)₂C₁₋₆ alkyl, NHSO₂C₁₋₆ alkyl, C(O)C₁₋₆ alkyl, C(O)OC₁₋₆ alkyl, NHC(O)C₁₋₆ alkyl, C(O)NHC₁₋₆ alkyl, C₃₋₆ cycloalkyl, 5-7 membered heterocycle, C₆₋₁₀ aryl, and C₅₋₁₀ heteroaryl;
A² is
a) a 5- or 6-membered heteroaryl ring which contains one, two, or three heteroatoms selected from O, N, or S; or
b) a 6-membered aromatic ring, which may contain one heteroatom;
R³ is selected from the group consisting of hydrogen, C₁₋₆ alkyl, C₂₋₆ alkenyl, C₂₋₆ alkynyl, C₁₋₆ alkoxy, halogen, C₁₋₆ haloalkyl, NO₂, CN, NH₂, NHC₁₋₆ alkyl, N(C₁₋₆ alkyl)₂, N₃, S(O)C₁₋₆ alkyl, S(O)₂C₁₋₆ alkyl, NHSO₂C₁₋₆ alkyl, C(O)C₁₋₆ alkyl, C(O)OC₁₋₆ alkyl, NHC(O)C₁₋₆ alkyl, C(O)NHC₁₋₆ alkyl, C₃₋₆ cycloalkyl, 5-7 membered heterocycle, C₆₋₁₀ aryl, and C₅₋₁₀ heteroaryl,
wherein
each C₃₋₆ cycloalkyl, 5-7 membered heterocycle, C₆₋₁₀ aryl, and C₅₋₁₀ heteroaryl is unsubstituted or substituted with one, two, or three (CH₂)₀₋₃R^{S};
and
each R^{S} is selected from the group consisting of C₁₋₆ alkyl, C₂₋₆ alkenyl, C₂₋₆ alkynyl, OH, C₁₋₆ alkoxy, halogen, C₁₋₆ haloalkyl, NO₂, CN, NH₂, NHC₁₋₆ alkyl, N(C₁₋₆ alkyl)₂, N₃, S(O)C₁₋₆ alkyl, S(O)₂C₁₋₆ alkyl, NHSO₂C₁₋₆ alkyl, C(O)C₁₋₆ alkyl, C(O)OC₁₋₆ alkyl, NHC(O)C₁₋₆ alkyl, C(O)NHC₁₋₆ alkyl, unsubstituted C₃₋₆ cycloalkyl, O-unsubstituted C₃₋₆ cycloalkyl, unsubstituted 5-7 membered heterocycle, unsubstituted C₆₋₁₀ aryl, and unsubstituted C₅₋₁₀ heteroaryl.

2. The compound of claim 1, wherein R¹ is C₁₋₁₀ alkyl, C₂₋₁₀ alkynyl, C₁₋₁₀ alkoxy, or C₆ aryl, which is optionally substituted with C₁₋₆ alkyl, C₂₋₆ alkenyl, C₂₋₆ alkynyl, C₁₋₆ alkoxy, (CH₂)₀₋₃C₃₋₆ cycloalkyl, or OC₃₋₆ cycloalkyl.

3. The compound of claim 1, wherein R¹ is C₁₋₁₀ alkoxy or C₆ aryl, which is optionally substituted with C₁₋₆ alkyl, C₂₋₆ alkenyl, C₂₋₆ alkynyl, C₁₋₆ alkoxy, or (CH₂)₀₋₃C₃₋₆ cycloalkyl.

4. The compound of claim 1, wherein A¹ is phenyl.

5. The compound of claim 1, wherein X is CH.

6. The compound of claim 5, wherein n2 is 1.

7. The compound of claim 6, wherein R²⁰ is OH.

8. The compound of claim 1, wherein X is N.

9. The compound of claim 8, wherein n2 is 1.

10. The compound of claim 9, wherein R²⁰ is CH(NH₂)C₁₋₆alkyl, CH(N[C₁₋₆ alkyl]₂)C₁₋₆alkyl, CH(NH₂)C₁₋₆alkyl-O—C₁₋₆alkyl, or CH(N[C₁₋₆ alkyl]₂)C₁₋₆alkyl-O—C₁₋₆alkyl.

11. The compound of claim 1, wherein R⁴ is H or C₁₋₆ alkyl.

12. The compound of claim 1, wherein L¹ is divalent C₃ cyclolakyl.

13. The compound of claim 1, wherein L¹ is [C(R^{L})₂]ₘ.

14. The compound of claim 13, wherein m is 1, one R^{L} is H and one R^{L} is C₁₋₆ alkyl.

15. The compound of claim 1, wherein A² is phenyl.

16. The compound of claim 1, wherein R³ is hydrogen or C₁₋₆ alkyl.

17. A compound or a pharmaceutically acceptable salt thereof selected from the group consisting of:

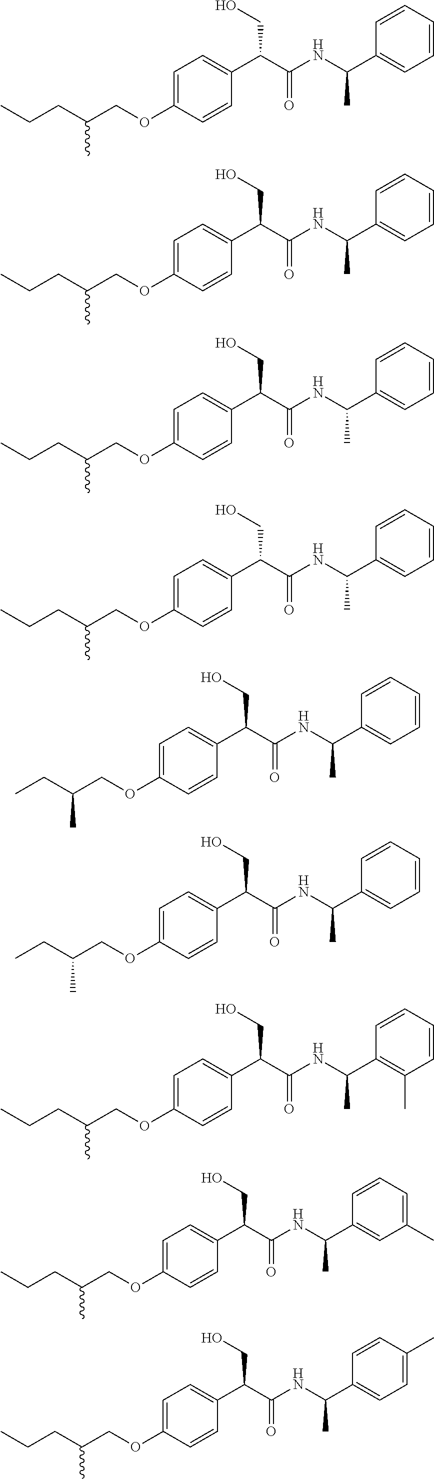

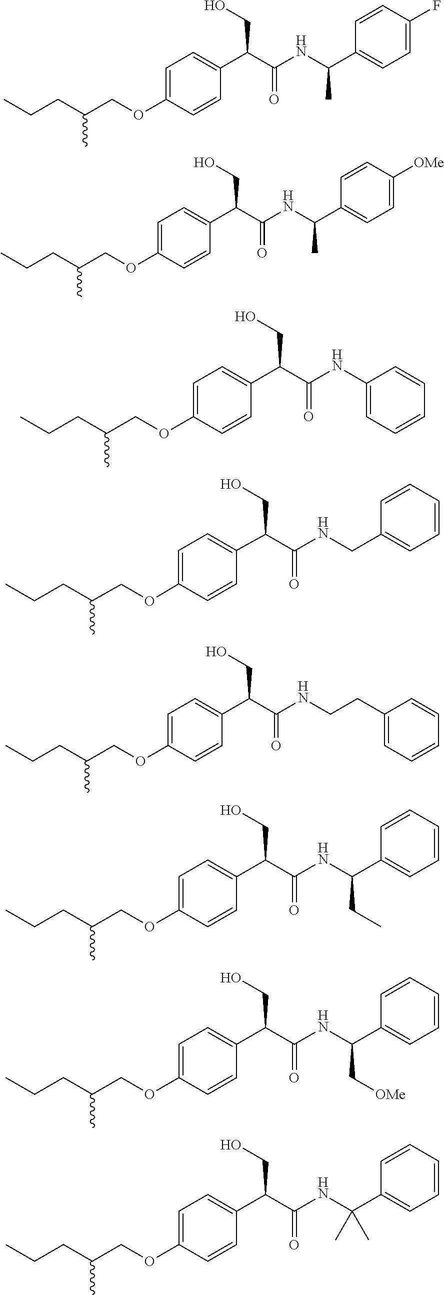

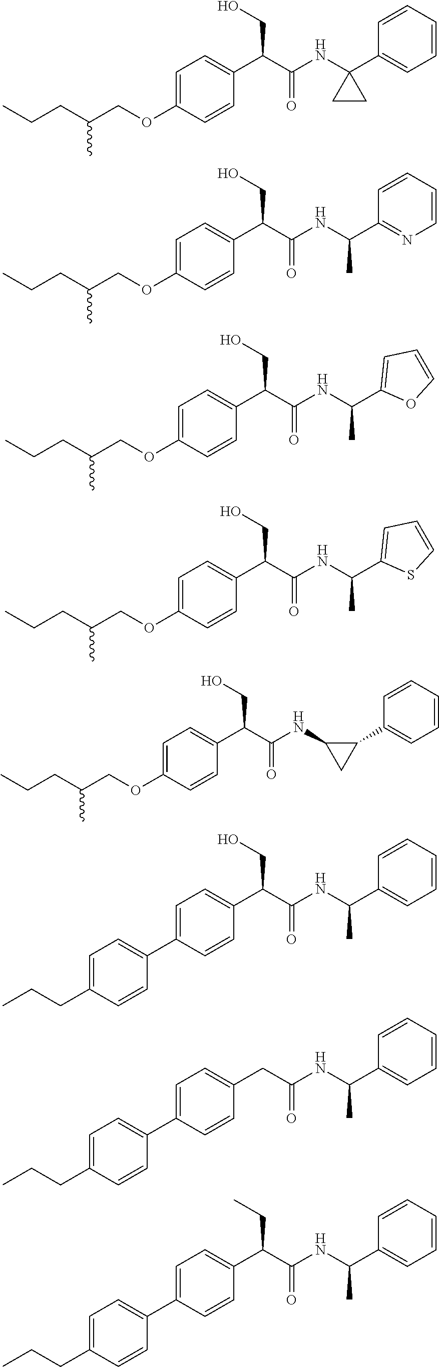

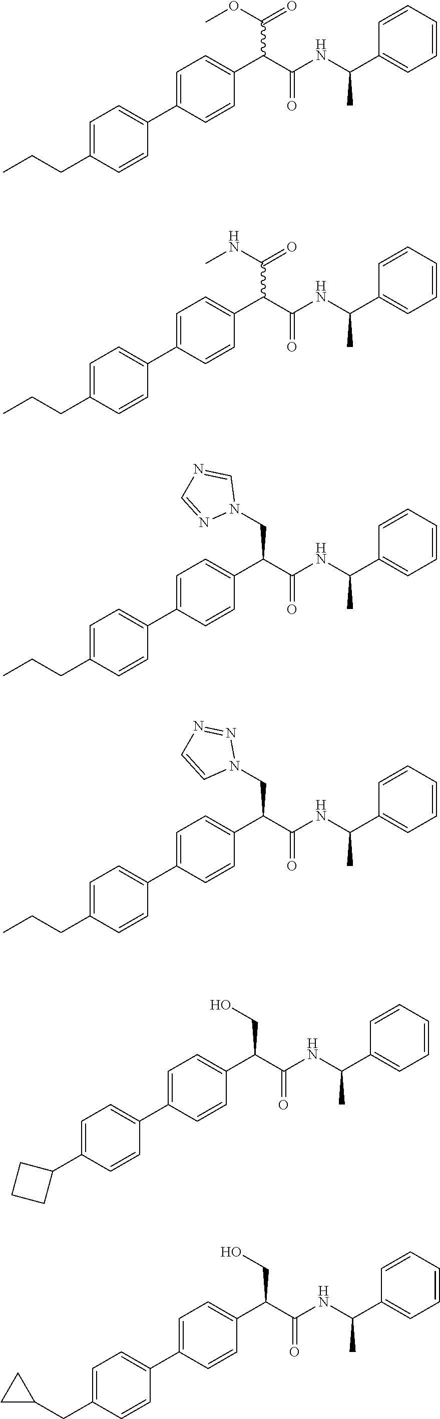 [SMILES: CCCc1ccc(-c2ccc(C(C(=O)N[C@H](C)c3ccccc3)C(=O)OC)cc2)cc1 ; CCCc1ccc(-c2ccc(C(C(=O)NC)C(=O)N[C@H](C)c3ccccc3)cc2)cc1 ; CCCc1ccc(-c2ccc([C@@H](Cn3cncn3)C(=O)N[C@H](C)c3ccccc3)cc2)cc1 ; CCCc1ccc(-c2ccc([C@@H](Cn3ccnn3)C(=O)N[C@H](C)c3ccccc3)cc2)cc1 ; C[C@@H](NC(=O)[C@H](CO)c1ccc(-c2ccc(C3CCC3)cc2)cc1)c1ccccc1 ; C[C@@H](NC(=O)[C@H](CO)c1ccc(-c2ccc(CC3CC3)cc2)cc1)c1ccccc1]

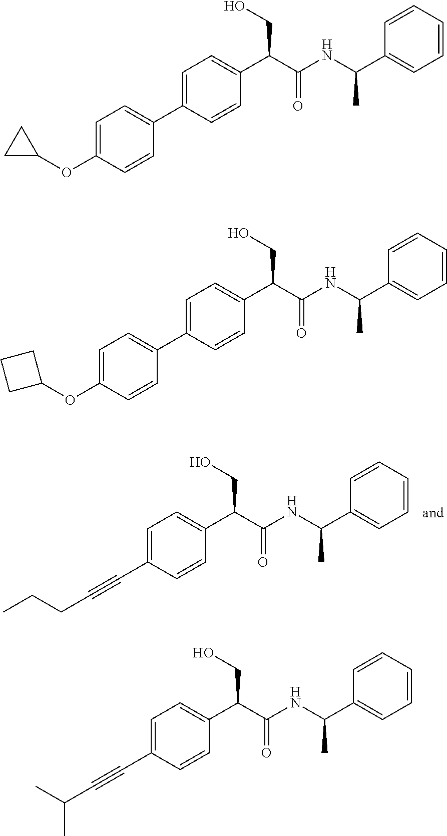 [SMILES: C[C@@H](NC(=O)[C@H](CO)c1ccc(-c2ccc(OC3CC3)cc2)cc1)c1ccccc1 ; C[C@@H](NC(=O)[C@H](CO)c1ccc(-c2ccc(OC3CCC3)cc2)cc1)c1ccccc1 ; CCCC#Cc1ccc([C@@H](CO)C(=O)N[C@H](C)c2ccccc2)cc1 ; CC(C)C#Cc1ccc([C@@H](CO)C(=O)N[C@H](C)c2ccccc2)cc1]

18. A compound or a pharmaceutically acceptable salt thereof selected from the group consisting of:

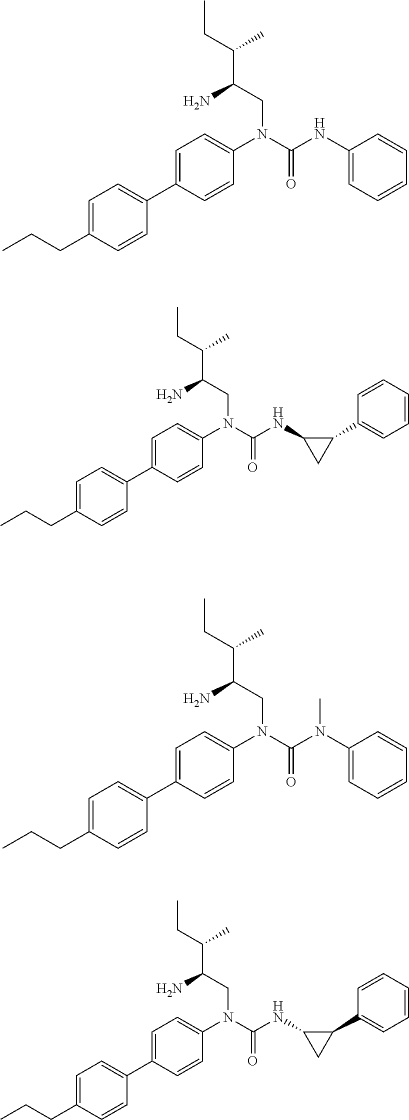 [SMILES: CCCc1ccc(-c2ccc(N(C[C@@H](N)[C@@H](C)CC)C(=O)Nc3ccccc3)cc2)cc1 ; CCCc1ccc(-c2ccc(N(C[C@@H](N)[C@@H](C)CC)C(=O)N[C@@H]3C[C@H]3c3ccccc3)cc2)cc1 ; CCCc1ccc(-c2ccc(N(C[C@@H](N)[C@@H](C)CC)C(=O)N(C)c3ccccc3)cc2)cc1 ; CCCc1ccc(-c2ccc(N(C[C@@H](N)[C@@H](C)CC)C(=O)N[C@H]3C[C@@H]3c3ccccc3)cc2)cc1]

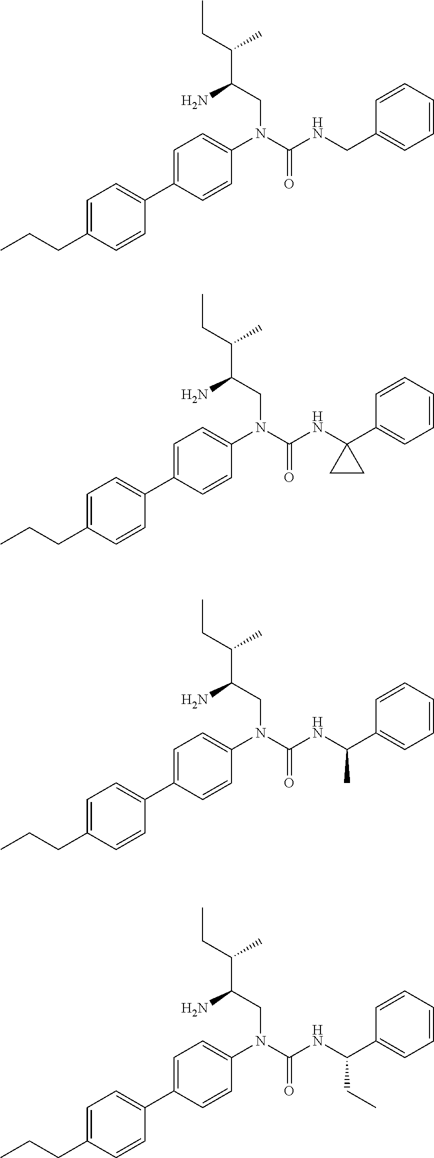 [SMILES: CCCc1ccc(-c2ccc(N(C[C@@H](N)[C@@H](C)CC)C(=O)NCc3ccccc3)cc2)cc1 ; CCCc1ccc(-c2ccc(N(C[C@@H](N)[C@@H](C)CC)C(=O)NC3(c4ccccc4)CC3)cc2)cc1 ; CCCc1ccc(-c2ccc(N(C[C@@H](N)[C@@H](C)CC)C(=O)N[C@H](C)c3ccccc3)cc2)cc1 ; CCCc1ccc(-c2ccc(N(C[C@@H](N)[C@@H](C)CC)C(=O)N[C@@H](CC)c3ccccc3)cc2)cc1]

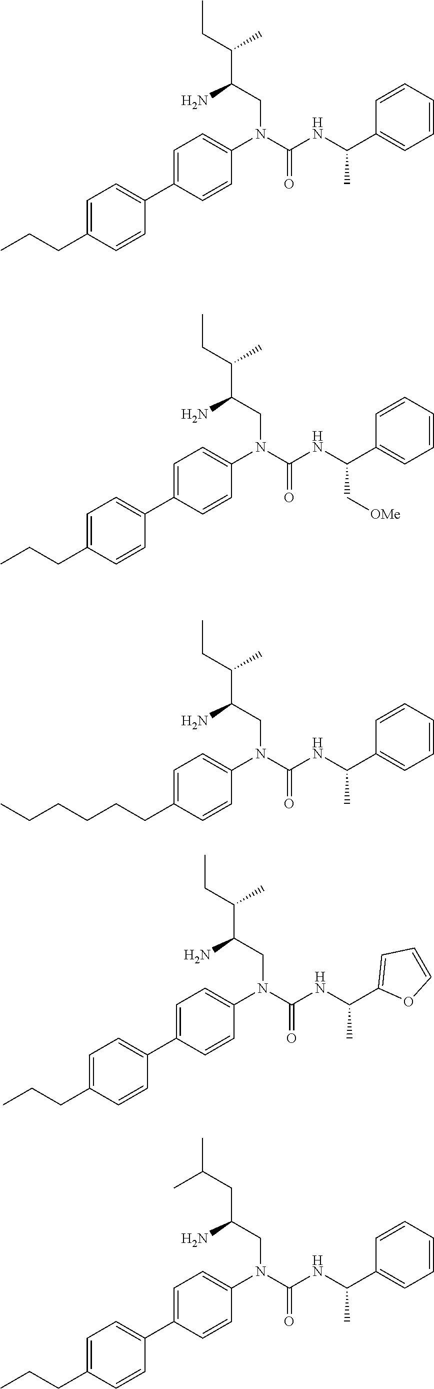

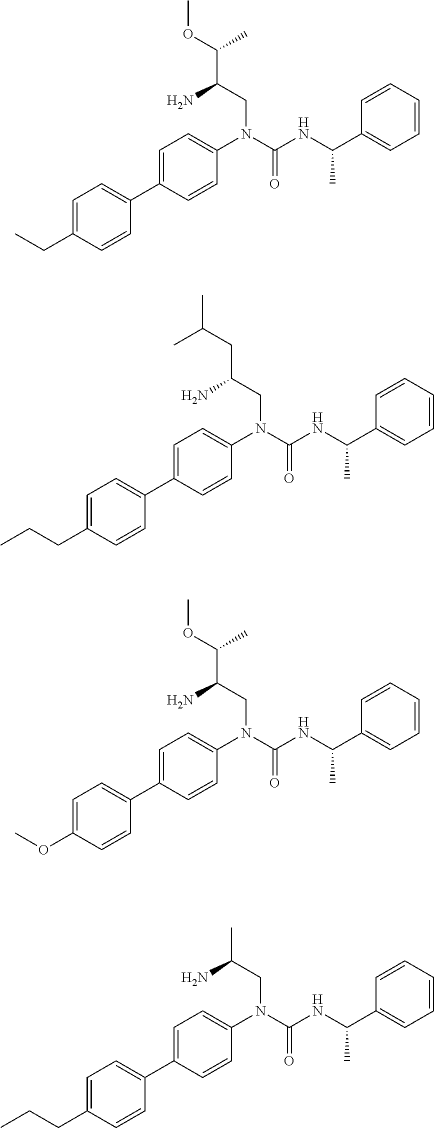 [SMILES: CCc1ccc(-c2ccc(N(C[C@@H](N)[C@@H](C)OC)C(=O)N[C@@H](C)c3ccccc3)cc2)cc1 ; CCCc1ccc(-c2ccc(N(C[C@H](N)CC(C)C)C(=O)N[C@@H](C)c3ccccc3)cc2)cc1 ; COc1ccc(-c2ccc(N(C[C@@H](N)[C@@H](C)OC)C(=O)N[C@@H](C)c3ccccc3)cc2)cc1 ; CCCc1ccc(-c2ccc(N(C[C@H](C)N)C(=O)N[C@@H](C)c3ccccc3)cc2)cc1]

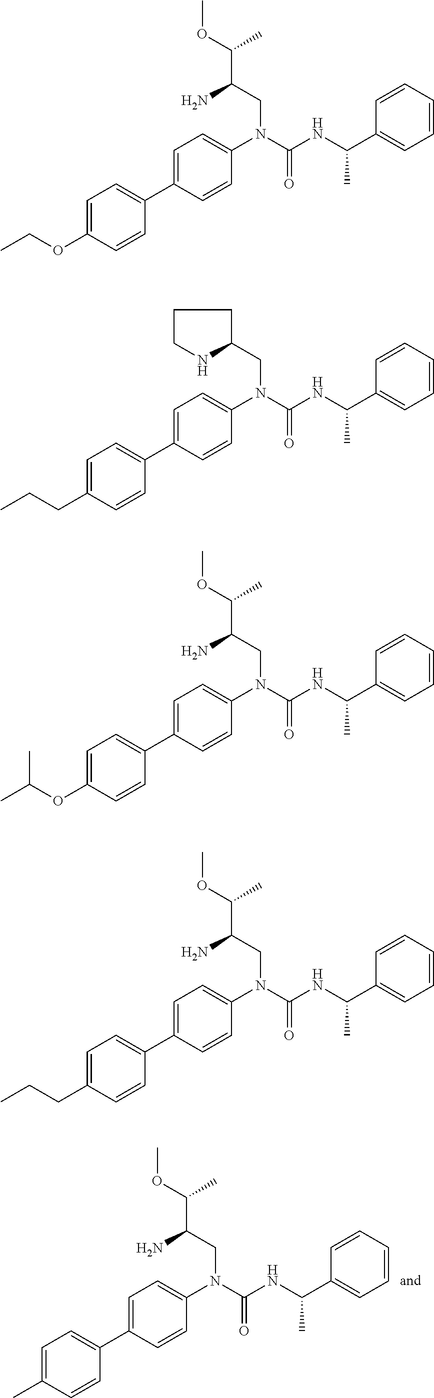 [SMILES: CCOc1ccc(-c2ccc(N(C[C@@H](N)[C@@H](C)OC)C(=O)N[C@@H](C)c3ccccc3)cc2)cc1 ; CCCc1ccc(-c2ccc(N(C[C@@H]3CCCN3)C(=O)N[C@@H](C)c3ccccc3)cc2)cc1 ; CO[C@H](C)[C@H](N)CN(C(=O)N[C@@H](C)c1ccccc1)c1ccc(-c2ccc(OC(C)C)cc2)cc1 ; CCCc1ccc(-c2ccc(N(C[C@@H](N)[C@@H](C)OC)C(=O)N[C@@H](C)c3ccccc3)cc2)cc1 ; CO[C@H](C)[C@H](N)CN(C(=O)N[C@@H](C)c1ccccc1)c1ccc(-c2ccc(C)cc2)cc1]

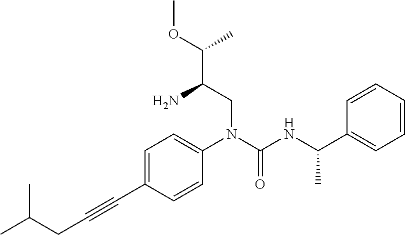 [SMILES: CO[C@H](C)[C@H](N)CN(C(=O)N[C@@H](C)c1ccccc1)c1ccc(C#CCC(C)C)cc1]

19. A method of treating a disease of disorder in a patient in needed thereof where modulation of the G protein-coupled receptor is beneficial comprising administering a compound of claim 1.

20.-21. (canceled)

22. The method of claim 19, to treat a neurological disorder, a metabolic disease, or an alcohol use disorder.

23.-26. (canceled)
